(19) United States
(12) Patent Application Publication
(10) Pub. No.: US 2021/0277015 A1
(43) Pub. Date: Sep. 9, 2021

(54) OGA INHIBITOR COMPOUNDS

(71) Applicant: Janssen Pharmaceutica NV, Beerse (BE)

(72) Inventors: José Manuel Bartolomé-Nebreda, Toledo (ES); Andrés Avelino Trabanco-Suárez, Olias del Rey (ES); Carlos Manuel Martinez-Viturro, Olias del Rey (ES); Francisca Delgado-Jiménez, Madrid (ES); Susana Conde-Ceide, Toledo (ES); Juan Antonio Vega Ramiro, Olias del Rey (ES)

(21) Appl. No.: 17/253,414

(22) PCT Filed: Jun. 20, 2019
(86) PCT No.: PCT/EP2019/066385

§ 371 Date: Dec. 17, 2020

(30) Foreign Application Priority Data
Jun. 20, 2018 (EP) 18382447,3

(51) Int. Cl. C07D 491/052; C07D 413/14; C07D 491/056; C07D 405/14; C07D 498/04; C07D 491/048; C07D 471/04

(52) U.S. Cl. CPC C07D 491/052; C07D 413/14; C07D 491/056; C07D 471/04; C07D 498/04; C07D 491/048; C07D 405/14

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

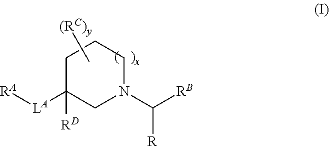

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA) O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in the N-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensible for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying 0-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyperphosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (Aβ) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2012/117219 (Summit Corp. plc., published 7 Sep. 2012) describes N-[[5-(hydroxymethyl)pyrrolidin-2-yl]methyl]alkylamide and N-alkyl-2-[5-(hydroxymethyl)pyrrolidin-2-yl]acetamide derivatives as OGA inhibitors. WO2014/159234 (Merck Patent GMBH, published 2 Oct. 2014) discloses mainly 4-phenyl or benzyl-piperidine and piperazine compounds substituted at the 1-position with an acetamido-thiazolylmethyl or acetamidoxazolylmethyl substituent and the compound N-[5-[(3-phenyl-1-piperidyl)methyl]thiazol-2-yl]acetamide; WO2016/0300443 (Asceneuron S.A., published 3 Mar. 2016), WO2017/144633 and WO2017/0114639 (Asceneuron S.A., published 31 Aug. 2017) disclose 1,4-disubstituted piperidines or piperazines as OGA inhibitors; WO2017/144637 (Asceneuron S.A, published 31 Aug. 2017) discloses more particular 4-substituted 1-[1-(1,3-benzodioxol-5-yl)ethyl]-piperazine; 1-[1-(2,3-dihydrobenzofuran-5-yl)ethyl]-; 1-[1-(2,3-dihydrobenzofuran-6-yl)ethyl]-; and 1-[1-(2,3-dihydro-1,4-benzodioxin-6-yl)ethyl]-piperazine derivatives as OGA inhibitors; WO2017/106254 (Merck Sharp & Dohme Corp.) describes substituted N-[5-[(4-methylene-1-piperidyl)methyl]thiazol-2-yl]acetamide compounds as OGA inhibitors.

There is still a need for OGA inhibitor compounds with an advantageous balance of properties, for example with improved potency, good bioavailability, pharmacokinetics, and brain penetration, and/or better toxicity profile. It is accordingly an object of the present invention to provide compounds that overcome at least some of these problems.

### SUMMARY OF THE INVENTION

The present invention is directed to compounds of Formula (I)

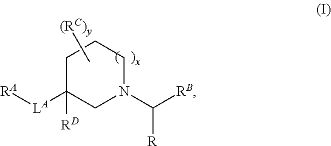

and the tautomers and the stereoisomeric forms thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —CH₂—, —O—, —OCH₂—, —CH₂O—, —NH—, —N(CH₃)—, —NHCH₂— and —CH₂NH—;
x represents 0;

### R is H or CH₃; and

R^{B} is a bicyclic radical of formula (b-1), (b-2) or (b-3)

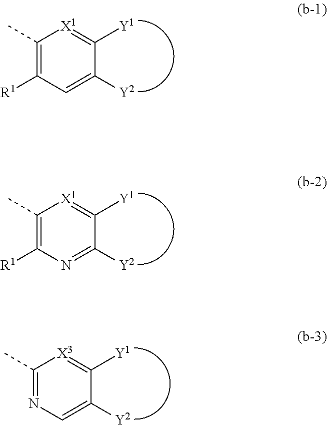

wherein
R¹ and R² are each selected from the group consisting of hydrogen, fluoro and methyl;
X¹, X² and X³ each represent CH, CF or N;
—Y¹—Y²— forms a bivalent radical selected from the group consisting of

| | | | |
|---|---|---|---|
| —O(CH₂)ₘO— | (c-1); | | |
| —O(CH₂)ₙ— | (c-2); | —(CH₂)ₙO— | (c-3); |
| —O(CH₂)ₚNR³— | (c-4); | —NR³(CH₂)ₚO— | (c-5); |
| —O(CH₂)(CO)NR³— | (c-6); | —NR³(CO)(CH₂)O— | (c-7); |
| —(CH₂)ₙNR³(CO)— | (c-8); | —(CO)NR³(CH₂)ₙ— | (c-9); and |
| —N═CH(CO)NR³— | (c-10); | | |

wherein
m is 1 or 2;
n and p each independently represent 2 or 3;
each R³ is independently H or C₁₋₄alkyl;
R^{C} is selected from the group consisting of fluoro, methyl, hydroxy, methoxy, trifluoromethyl, and difluoromethyl;
R^{D} is selected from the group consisting of hydrogen, fluoro, methyl, hydroxy, methoxy, trifluoromethyl, and difluoromethyl; and
y represents 0, 1 or 2;
with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{D};
c) R^{D} is not hydroxy or methoxy when L^{A} is —O—, —OCH₂—, —CH₂O—, —NH—, —N(CH₃)—, —NH(CH₂)— or —(CH₂)NH—;
   and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a therapeutically effective amount of any of the compounds or pharmaceutical compositions described above.

Further exemplifying the invention are methods of inhibiting OGA, comprising administering to a subject in need thereof a prophylactically or a therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I), as defined herein before, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or may be useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In a particular embodiment, the invention is directed to compounds of Formula (I) as referred to herein, and the tautomers and the stereoisomeric forms thereof, wherein

R^{A} is a heteroaryl radical selected from the group consisting of 3-pyridinyl, pyridin-4-yl, pyrimidin-4-yl, and pyrazin-2-yl, each of which may be optionally substituted with 1, 2 or 3 substituents each independently selected from the group consisting of halo; C₁₋₄alkyl optionally substituted with 1, 2, or 3 independently selected halo substituents;
and C₁₋₄alkyloxy optionally substituted with 1, 2, or 3 independently selected halo substituents;
and the pharmaceutically acceptable salts and the solvates thereof.

In a further embodiment, the invention is directed to compounds of Formula (I) as referred to herein, and the tautomers and the stereoisomeric forms thereof, wherein

R^{A} is a heteroaryl radical selected from the group consisting of pyridin-4-yl, pyrimidin-4-yl, and pyrazin-2-yl, each of which may be optionally substituted with 1, 2 or 3 substituents each independently selected from the group consisting of halo; C₁₋₄alkyl optionally substituted with 1, 2, or 3 independently selected halo substituents; and
C₁₋₄alkyloxy optionally substituted with 1, 2, or 3 independently selected halo substituents;
and the pharmaceutically acceptable salts and the solvates thereof.

In a further embodiment, the invention is directed to compounds of Formula (I) as referred to herein, and the tautomers and the stereoisomeric forms thereof, wherein

R^{A} is a heteroaryl radical selected from the group consisting of pyridin-4-yl, pyrimidin-4-yl, and pyrazin-2-yl, each of which may be optionally substituted with 1, 2 or 3 substituents each independently selected from the group consisting of C₁₋₄alkyl optionally substituted with 1, 2, or 3 independently selected halo substituents; and C₁₋₄alkyloxy optionally substituted with 1, 2, or 3 independently selected halo substituents;
and the pharmaceutically acceptable salts and the solvates thereof.

In an additional embodiment, the invention is directed to compounds of Formula (I) as referred to herein, and the tautomers and the stereoisomeric forms thereof, wherein

R^{A} is pyridin-4-yl or pyrimidin-4-yl, each of which may be optionally substituted with 1, 2 or 3 substituents each independently selected from the group consisting of C₁₋₄alkyl optionally substituted with 1, 2, or 3 independently selected halo substituents; and C₁₋₄alkyloxy optionally substituted with 1, 2, or 3 independently selected halo substituents.

In an additional embodiment, the invention is directed to compounds of Formula (I) as referred to herein, and the tautomers and the stereoisomeric forms thereof, wherein

R^{A} is a heteroaryl radical selected from the group consisting of 3-pyridinyl and pyridin-4-yl, each of which is substituted with 1 or 2 independently selected C₁₋₄alkyl substituents;
and the pharmaceutically acceptable salts and the solvates thereof.

In a further embodiment, the invention is directed to compounds of Formula (I), as referred to herein, and the tautomers and the stereoisomeric forms thereof, wherein L^{A} is selected from the group consisting of a covalent bond, —CH₂—, —O—, —OCH₂—, —CH₂O—, and —NHCH₂—.

In a further embodiment, the invention is directed to compounds of Formula (I), as referred to herein, and the tautomers and the stereoisomeric forms thereof, wherein L^{A} is selected from the group consisting of a covalent bond, —CH₂—, —O—, —OCH₂—, and —CH₂O—.

In a further embodiment, the invention is directed to compounds of Formula (I), as referred to herein, and the tautomers and the stereoisomeric forms thereof, wherein L^{A} is selected from the group consisting of —CH₂—, —O—, —OCH₂—, —CH₂O—, —NH—, —N(CH₃)—, —NHCH₂— and —CH₂NH—.

In another embodiment, the invention is directed to compounds of Formula (I), as referred to herein, and the tautomers and the stereoisomeric forms thereof, wherein L^{A} is selected from the group consisting of —CH₂—, —O—, —OCH₂—, —CH₂O—, and —NHCH₂—.

In a further embodiment, the invention is directed to compounds of Formula (I), as referred to herein, and the tautomers and the stereoisomeric forms thereof, wherein L^{A} is —O— or —OCH₂—.

In yet another embodiment, the invention is directed to compounds of Formula (I), as referred to herein, and the tautomers and the stereoisomeric forms thereof, wherein R^{B} is a bicyclic radical of formula (b-1) or (b-2).

In a further embodiment, the invention is directed to compounds of Formula (I), as referred to herein, and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen; X¹ is N or CH; and X² is CH.

In another embodiment, the invention is directed to compounds of Formula (I), as referred to herein, and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen; X¹ is N or CH; X² is CH; and —Y¹—Y²— forms a bivalent radical selected from the group consisting of (c-1), (c-2), (c-4), (c-5), (c-6) and (c-9), in particular, (c-1), (c-2), (c-4), (c-5) and (c-9).

In another embodiment, the invention is directed to compounds of Formula (I), as referred to herein, and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen; X¹ is N or CH; X² is CH; and —Y¹—Y²— forms a bivalent radical selected from the group consisting of (c-1), (c-2), (c-4) and (c-6).

In a further embodiment, the invention is directed to compounds of Formula (I), and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen; X¹ is N or CH; X² is CH; and —Y¹—Y²— forms a bivalent radical selected from the group consisting of (c-1), (c-2), (c-4) and (c-6), wherein m is 2; n is 2 or 3; and p is 2.

In a further embodiment, the invention is directed to compounds of Formula (I), and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen; X¹ is N or CH; X² is CH; and —Y¹—Y²— forms a bivalent radical selected from the group consisting of (c-1), (c-2), (c-4), (c-5) and (c-9), wherein m is 2; n is 2 or 3; and p is 2.

In a further embodiment, the invention is directed to compounds of Formula (I), as referred to herein, and the tautomers and the stereoisomeric forms thereof, wherein R^{D} is selected from the group consisting of hydrogen, fluoro, and methyl.

In a further embodiment, the invention is directed to compounds of Formula (I), as referred to herein, and the tautomers and the stereoisomeric forms thereof, wherein R^{D} is hydrogen or methyl.

In a further embodiment, the invention is directed to compounds of Formula (I), as referred to herein, and the tautomers and the stereoisomeric forms thereof, wherein y represents 0 or 1.

In a further embodiment, the invention is directed to compounds of Formula (I), as referred to herein, and the tautomers and the stereoisomeric forms thereof, wherein y represents 0.

In a further embodiment, the invention is directed to compounds of Formula (I), as referred to herein, and the tautomers and the stereoisomeric forms thereof, wherein y represents 1.

In another embodiment, the invention is directed to compounds of Formula (I), as referred to herein, and the tautomers and the stereoisomeric forms thereof, wherein R^{A} is a heteroaryl radical selected from the group consisting of pyridin-4-yl, pyrimidin-4-yl, and pyrazin-2-yl, each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents; L^{A} is selected from the group consisting of a covalent bond, —CH₂—, —O—, —OCH₂—, —CH₂O—, and —NHCH₂—;

x represents 0;

### R is H or CH₃; and

R^{B} is a bicyclic radical of formula (b-1) or (b-2), wherein
R¹ and R² are each selected from the group consisting of hydrogen, fluoro and methyl;
X¹, X² and X³ each represent CH, CF or N;
—Y¹—Y²— forms a bivalent radical selected from the group consisting of (c-1), (c-2), (c-4) and (c-6); wherein
m is 1 or 2;
n and p each independently represent 2 or 3;
each R³ is independently H or C₁₋₄alkyl;
R^{C} is fluoro or methyl;
R^{D} is selected from the group consisting of hydrogen, fluoro, and methyl; and
y represents 0 or 1;
and the pharmaceutically acceptable salts and the solvates thereof.

In a further embodiment, the invention is directed to compounds of Formula (I), and the tautomers and the stereoisomeric forms thereof, wherein R^{B} is selected from the group consisting of

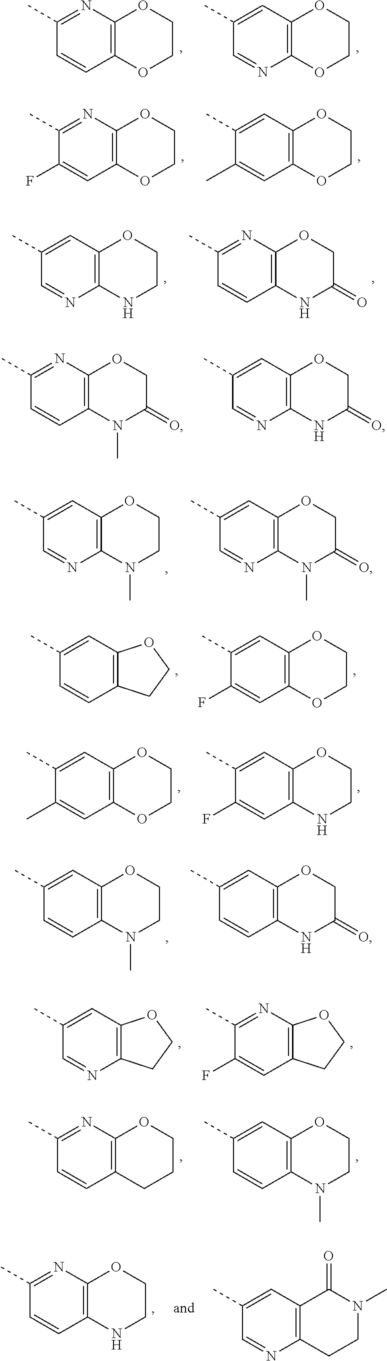

In a further embodiment, the invention is directed to compounds of Formula (I), and the tautomers and the stereoisomeric forms thereof, wherein R^{B} is selected from the group consisting of

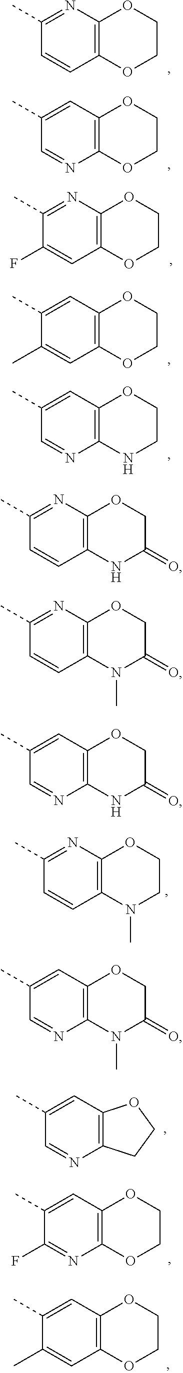

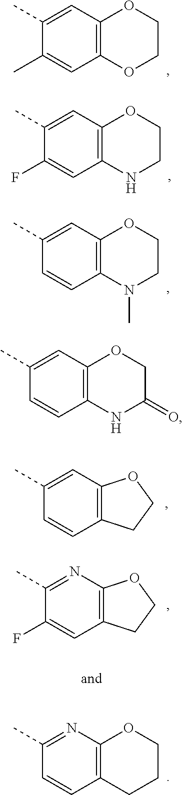

In a further embodiment, the invention is directed to compounds of Formula (I), and the tautomers and the stereoisomeric forms thereof, wherein R^{B} is selected from the group consisting of

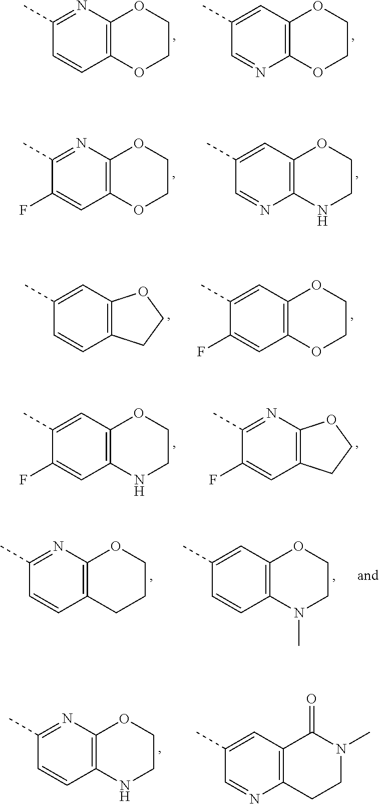

In a further embodiment, the invention is directed to compounds of Formula (I), and the tautomers and the stereoisomeric forms thereof, wherein R^{B} is selected from the group consisting of

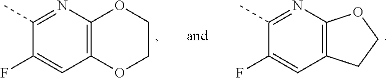

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C₁₋₄alkyl is as defined before. When reference is made to L^{A}, the definition is to be read from left to right, with the left part of the linker bound to R^{A} and the right part of the linker bound to the pyrrolidinediyl or piperidinediyl ring. Thus, when L^{A} is, for example, —O—CH₂—, then R^{A}-L^{A}-is R^{A}—O—CH₂—. When R^{C} is present more than once, where possible, it may be bound at the same carbon atom of the pyrrolidinediyl or piperidinediyl ring, and each instance may be different.

In general, whenever the term “substituted” is used in the present invention, it is meant, unless otherwise indicated or is clear from the context, to indicate that one or more hydrogens, in particular 1 to 3 hydrogens, preferably 1 or 2 hydrogens, more preferably 1 hydrogen, on the atom or radical indicated in the expression using “substituted” are replaced with a selection of substituents from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Experimental Procedure 1

The final compounds of Formula (I-a) can be prepared by reacting an intermediate compound of Formula (II) with a compound of Formula (XV) according to reaction scheme (1). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, triethylamine, and/or a Lewis acid, such as, for example titanium tetraisopropoxide or titanium tetrachloride, under thermal conditions, such as, 0° C. or room temperature, or 140° C., for example for 1 hour or 24 hours. In reaction scheme (1) all variables are defined as in Formula (I).

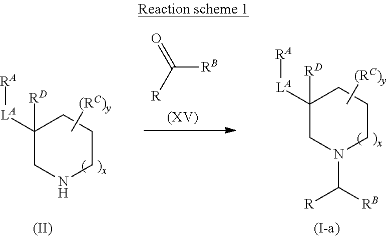

### Experimental Procedure 2

Additionally final compounds of Formula (I-a) can be prepared by reacting an intermediate compound of Formula (II) with a compound of Formula (XVI) according to reaction scheme (2). The reaction is performed in a suitable reaction-inert solvent, such as, for example, acetonitrile, a suitable base, such as, for example, triethylamine or diisopropylethylamine, under thermal conditions, such as, 0° C. or room temperature, or 75° C., for example for 1 hour or 24 hours. In reaction scheme (2) all variables are defined as in Formula (I), and wherein halo is chloro, bromo or iodo.

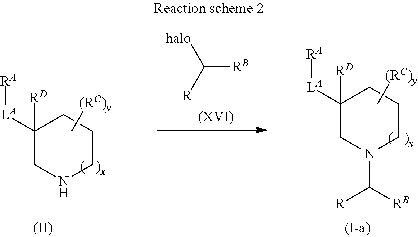

### Experimental Procedure 3

Additionally, final compounds of Formula (I), wherein R is CH₃, herein referred to as (I-b), can be prepared by reacting an intermediate compound of Formula (II) with a compound of Formula (XVII) followed by reaction of the formed imine derivative with and intermediate compound of Formula (XVIII) according to reaction scheme (3). The reaction is performed in a suitable reaction-inert solvent, such as, for example, anhydrous dichloromethane, a Lewis acid, such as, for example titanium tetraisopropoxide or titanium tetrachloride, under thermal conditions, such as, 0° C. or room temperature, for example for 1 hour or 24 hours. In reaction scheme (3) all variables are defined as in Formula (I), and wherein halo is chloro, bromo or iodo

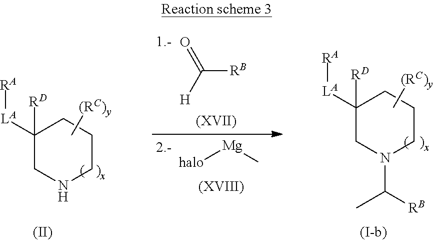

### Experimental Procedure 4

Additionally final compounds of Formula (I) wherein L^{A} is —NH—CH₂—, herein referred to as (I-c), can be prepared by reacting an intermediate compound of Formula (III) with a compound of Formula (V) according to reaction scheme (4). The reaction is performed in the presence of a palladium catalyst, such as, for example tris(dibenzylideneacetone)dipalladium(0), a ligand, such as, for example 2-dicyclohexylphosphino-2′-(N,N-dimethylamino)biphenyl, a base, such as, for example sodium tert-butoxide, a suitable reaction-inert solvent, such as, for example, anhydrous 1,4-dioxane, under thermal conditions, such as, 100° C., for example for 4 hour or 24 hours. In reaction scheme (4) all variables are defined as in Formula (I), and wherein halo is chloro, bromo or iodo

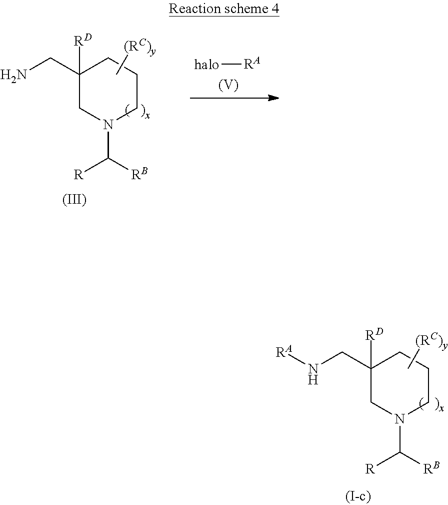

### Experimental Procedure 5

Intermediate compounds of Formula (II) can be prepared cleaving a protecting group in an intermediate compound of Formula (IV) according to reaction scheme (5). In reaction scheme (5) all variables are defined as in Formula (I), and PG is a suitable protecting group of the nitrogen function such as, for example, tert-butoxycarbonyl (Boc), ethoxycarbonyl, benzyl, benzyloxycarbonyl (Cbz). Suitable methods for removing such protecting groups are widely known to the person skilled in the art and comprise but are not limited to: Boc deprotection: treatment with a protic acid, such as, for example, trifluoroacetic acid, in a reaction inert solvent, such as, for example, dichloromethane; ethoxycarbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol.

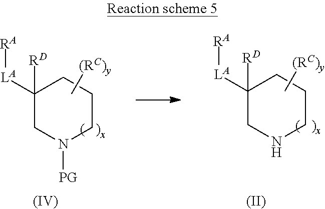

### Experimental Procedure 6

Intermediate compounds of Formula (IV-a) can be prepared by “Negishi coupling” reaction of a halo compound of Formula (V) with an organozinc compound of Formula (VI) according to reaction scheme (6). The reaction is performed in a suitable reaction-inert solvent, such as, for example, tetrahydrofuran, and a suitable catalyst, such as, for example, Pd(OAc)₂, a suitable ligand for the transition metal, such as, for example, 2-dicyclohexylphosphino-2′,6′-diisopropoxybiphenyl [CAS: 787618-22-8], under thermal conditions, such as, for example, room temperature, for example for 1 hour. In reaction scheme (6) all variables are defined as in Formula (I), L^{A} is a bond or CH₂ and halo is preferably bromo or iodo. PG is defined as in Formula (IV).

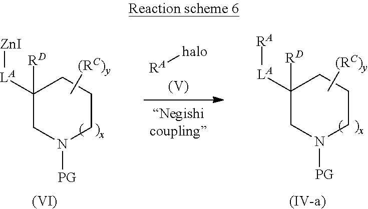

### Experimental Procedure 7

Intermediate compounds of Formula (VI) can be prepared by reaction of a halo compound of Formula (VII) with zinc according to reaction scheme (7). The reaction is performed in a suitable reaction-inert solvent, such as, for example, tetrahydrofuran, and a suitable salt, such as, for example, lithium chloride, under thermal conditions, such as, for example, 40° C., for example in a continuous-flow reactor. In reaction scheme (7) all variables are defined as in Formula (I), L^{A} is a bond or CH₂ and halo is preferably iodo. PG is defined as in Formula (IV).

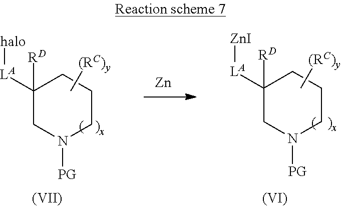

### Experimental Procedure 8

Intermediate compounds of Formula (IV) wherein R^{D} is H, herein referred to as (IV-b), can be prepared by hydrogenation reaction of an alkene compound of Formula (VIII) according to reaction scheme (8). The reaction is performed in a suitable reaction-inert solvent, such as, for example, methanol, and a suitable catalyst, such as, for example, palladium on carbon, and hydrogen, under thermal conditions, such as, for example, room temperature, for example for 3 hours. In reaction scheme (8) all variables are defined as in Formula (I) and PG is defined as in Formula (IV).

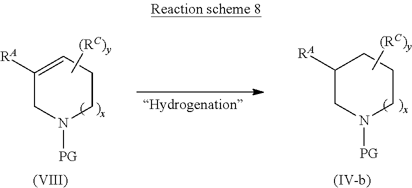

### Experimental Procedure 9

Intermediate compounds of Formula (VIII) can be prepared by “Suzuki coupling” reaction of an alkene compound of Formula (IX) and a halo derivative of Formula (V) according to reaction scheme (9). The reaction is performed in a suitable reaction-inert solvent, such as, for example, 1,4-dioxane, and a suitable catalyst, such as, for example, tetrakis(triphenylphosphine)palladium(0), a suitable base, such as, for example, NaHCO₃ (aq. sat. soltn.), under thermal conditions, such as, for example, 130° C., for example for 30 min under microwave irradiation. In reaction scheme (9) all variables are defined as in Formula (I), halo is preferably bromo or iodo, L^{A} is a bond, and PG is defined as in Formula (IV), L^{A} is a bond and R^{D} is H.

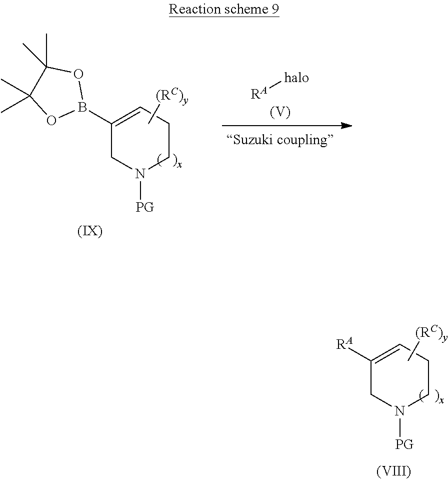

### Experimental Procedure 10

Intermediate compounds of Formula (IV-c) can be prepared by reaction of a hydroxy compound of Formula (X) and a halo derivative of Formula (V) according to reaction scheme (10). The reaction is performed in a suitable reaction-inert solvent, such as, for example, dimethylformamide or dimethylsulfoxide, and a suitable base, such as, sodium hydride or potassium tert-butoxide, under thermal conditions, such as, for example, 50° C., for example for 48 hours. In reaction scheme (10) all variables are defined as in Formula (I), L^{A′} is a bond or CH₂ and halo is preferably chloro, bromo or fluoro. PG is defined as in Formula (IV).

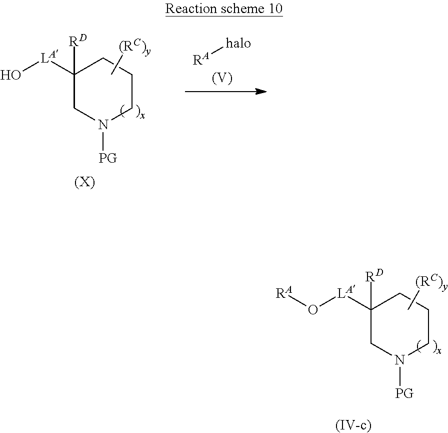

### Experimental Procedure 11

Alternatively intermediate compounds of Formula (IV-c) can be prepared by “Mitsunobu reaction” of a hydroxy compound of Formula (X) and a hydroxy derivative of Formula (XI) according to reaction scheme (11). The reaction is performed in a suitable reaction-inert solvent, such as, for example, toluene, a phosphine, such as, triphenylphosphine, a suitable coupling agent, such as, for example DIAD (CAS: 2446-83-5), under thermal conditions, such as, for example, 70° C., for example for 17 hours. In reaction scheme (11) all variables are defined as in Formula (I), L^{A′} is a bond or CH₂ and halo is preferably chloro, bromo or fluoro. PG is defined as in Formula (IV).

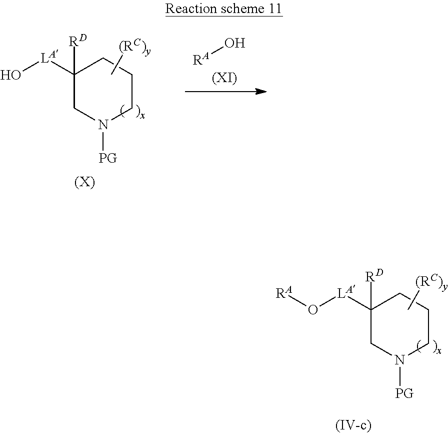

### Experimental Procedure 12

Intermediate compounds of Formula (III) can be prepared cleaving the protecting group in an intermediate compound of Formula (XI) according to reaction scheme (12). The reaction is performed in the presence of hydrazine hydrate in a suitable reaction-inert solvent, such as, for example, ethanol, under thermal conditions, such as, for example, 80° C., for example for 2 hours. In reaction scheme (12) all variables are defined as in Formula (I).

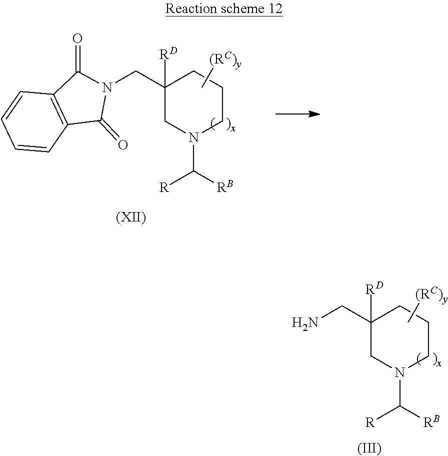

### Experimental Procedure 13

Intermediate compounds of Formula (XII) can be prepared by reacting an intermediate compound of Formula (XIII) with phthalimide according to reaction scheme (13). The reaction is performed in the presence of a phosphine, such as, for example triphenylphosphine, a suitable coupling agent, such as, for example diisopropyl azodicarboxylate in a suitable reaction-inert solvent, such as, for example, dry tetrahydrofuran, under thermal conditions, such as, for example, room temperature, for example for 24 hours. In reaction scheme (13) all variables are defined as in Formula (I).

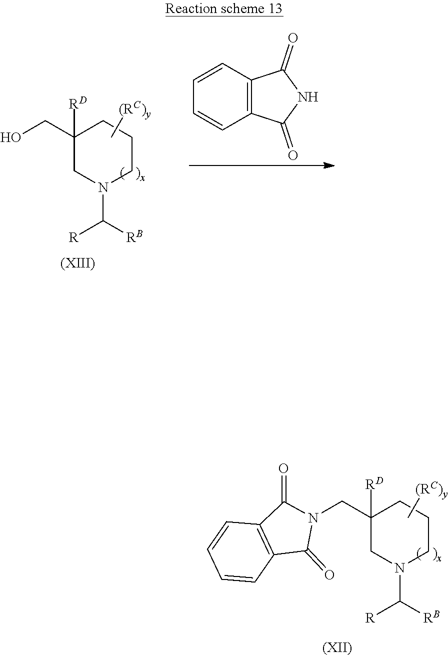

### Experimental Procedure 14

Intermediate compounds of Formula (XIII) can be prepared by deprotecting the alcohol group in an intermediate compound of Formula (XIV) according to reaction scheme (14). The reaction is performed in the presence of a fluoride source, such as, for example tetrabutylammonium fluoride, in a suitable reaction-inert solvent, such as, for example, dry tetrahydrofuran, under thermal conditions, such as, for example, room temperature, for example for 16 hours. In reaction scheme (13) all variables are defined as in Formula (I) and PG¹ is selected from the group consisting of trimethylsilyl, tert-butyldimethylsilyl, triisopropylsilyl or tert-butyldiphenylsilyl.

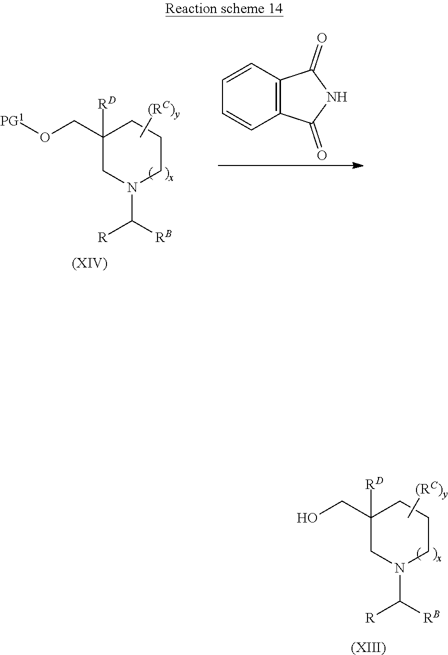

Intermediates of Formulae (V), (VII), (IX), (XV), (XVI), (XVII) and (XVIII) are commercially available or can be prepared by know procedures to those skilled in the art.

### Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical States in Alzheimer's and Tauopathy Diseases:

In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that Aβ accumulation and tau-aggregation begins many years before the onset of overt clinical impairment. The key risk factors for elevated amyloid accumulation, tau-aggregation and development of AD are age (i.e, 65 years or older), APOE genotype, and family history. Approximately one third of clinically normal older individuals over 75 years of age demonstrate evidence of Aβ or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at-risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

### Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The invention also provides a kit comprising a compound according to the invention, prescribing information also known as “leaflet”, a blister package or bottle, and a container. Furthermore, the invention provides a kit comprising a pharmaceutical composition according to the invention, prescribing information also known as “leaflet”, a blister package or bottle, and a container. The prescribing information preferably includes advice or instructions to a patient regarding the administration of the compound or the pharmaceutical composition according to the invention. In particular, the prescribing information includes advice or instruction to a patient regarding the administration of said compound or pharmaceutical composition according to the invention, on how the compound or the pharmaceutical composition according to the invention is to be used, for the prevention and/or treatment of a tauopathy in a subject in need thereof. Thus, in an embodiment, the invention provides a kit of parts comprising a compound of Formula (I) or a stereoisomeric for thereof, or a pharmaceutically acceptable salt or a solvate thereof, or a pharmaceutical composition comprising said compound, and instructions for preventing or treating a tauopathy. The kit referred to herein can be, in particular, a pharmaceutical package suitable for commercial sale.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### Experimental Part

Hereinafter, the term “m.p.” means melting point, “min” means minutes, “ACN” or “CH₃CN” mean acetonitrile, “aq.” means aqueous, “Boc” means tert-butyloxycarbonyl, “DMF” means dimethylformamide, “r.t.” or “RT” means room temperature, “rac” or “RS” means racemic, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “iPrOH” means isopropyl alcohol, “RP” means reversed phase, “Rₜ” means retention time (in minutes), “[M+H]⁺” means the protonated mass of the free base of the compound, “wt” means weight, “THF” means tetrahydrofuran, “Et₂O” means diethylether, “EtOAc” means ethyl acetate, “DCM” means dichloromethane, “DBAD” means di-tert-butyl azodicarboxylate, “DIPEA” means N,N-diisopropylethylamine, “DCE” means 1,2-dichloroethane, “MeOH” means methanol, “sat” means saturated, “soltn” or “sol.” means solution, “EtOH” means ethanol, “TFA” means trifluoroacetic acid, “2-meTHF” or “Me-THF” means 2-methyl-tetrahydrofuran, “NMP” means N-methylpyrrolidone, “Pd(OAc)₂” or “(OAc)₂Pd” means palladium(II) acetate, “Pd₂(dba)₃” means tris(dibenzylideneacetone)dipalladium(0), Pd(PPh₃)₄” means tetrakis(triphenylphosphine)palladium(0), “PdCl₂(dppf)” means [1,1′-bis(diphenylphosphino)ferrocene]dichloropalladium(II), “PdCl₂(PPh₃)₂” means bis(triphenylphosphine)palladium(II) dichloride, “Pd(t-Bu₃P)₂” means bis(tert-butylphosphine)palladium(0), “PdCl₂(dtbpf)” means [1,1′-bis(di-tert-butylphosphino)ferrocene]dichloropalladium(II), “RuPhos” means 2-dicyclohexylphosphino-2′,6′-diisopropoxybiphenyl, and “TMSCl” means trimethylsilyl chloride.

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “R*” or “S*” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

Flow chemistry reactions were performed in a Vapourtec R2+R4 unit using standard reactors provided by the vendor.

Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques.

Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or PuriFlash®430evo systems from Interchim, or 971-FP systems from Agilent, or Isolera 1SV systems from Biotage.

### Preparation of the Intermediates

### Preparation of Intermediate 1

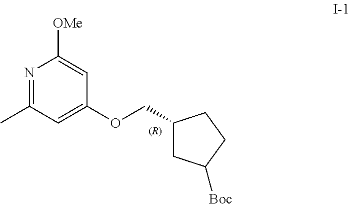

NaH (60% dispersion in mineral oil, 238 mg, 5.96 mmol) was added to a solution of (R)-3-hydroxymethyl-pyrrolidine-1-carboxylic acid tert-butyl ester (CAS: 138108-72-2; 1.00 g, 4.97 mmol) in DMF (10 mL) at 0° C. under N₂ atmosphere. The mixture was stirred at 0° C. for 15 min, and 4-bromo-2-methoxy-6-methylpyridine (CAS: 1083169-00-9; 1.15 g, 5.47 mmol) was added dropwise. The reaction mixture was stirred at 0° C. for 1 h and then at 70° C. for 20 h. The reaction was quenched with NH₄Cl (sat., aq.) and extracted with heptane. The organic layer was dried (MgSO₄), filtered and evaporated in vacuo. The crude mixture was purified by flash column chromatography (SiO₂, EtOAc in heptane, gradient from 100:0 to 50:50) to afford intermediate 1 (970 mg, 61%).

### Preparation of Intermediate 2

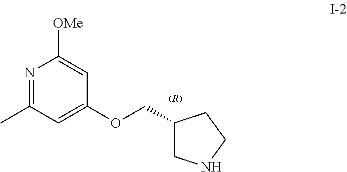

A solution of intermediate 1 (970 mg, 3.01 mmol) in MeOH (30 mL) was added to a closed reactor containing Amberlyst®15 hydrogen form (CAS: 39389-20-3; 3.20 g, 15.0 mmol). The mixture was shaken in a solid phase reactor at room temperature for 16 h. The resin was washed with MeOH (the fraction was discarded), then with NH₃ (7N in MeOH). The filtrates were concentrated in vacuo to give intermediate 2 (600 mg, 90%) as a pale brown oil.

### Preparation of Intermediate 3

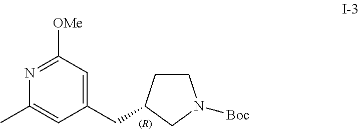

A 0.32 M solution of intermediate 72 in THF (34 mL, 10.9 mmol) was added to a flask containing 4-bromo-6-methyl pyridine-2-ol (CAS: 865156-59-8; 2.00 g, 9.89 mmol) under N₂ atmosphere. TMEDA (CAS: 110-18-9; 1.63 mL, 10.9 mmol) and Pd(PPh₃)₂Cl₂ (417 mg, 0.59 mmol) were successively added. The reaction mixture was stirred at 60° C. for 1 h. Then, the mixture was quenched with a 1/1 solution of NH₄Cl (sat.) and NH₃ (26% aq.) and extracted with EtOAc. The organic layer was dried (MgSO₄), filtered and the solvent was evaporated in vacuo. The crude product was purified by flash column chromatography (SiO₂, EtAOc in DCM, gradient from 0/100 to 40/60). The desired fractions were collected and concentrated in vacuo to afford intermediate 3 (2.49 g, 82%) as an oil.

### Preparation of Intermediate 4

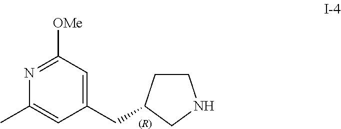

A solution of intermediate 3 (2.49 g, 8.13 mmol) in MeOH (70 mL) was added to a closed reactor containing Amberlyst®15 hydrogen form (CAS: 39389-20-3; 8.65 g, 40.6 mmol). The mixture was shaken in a solid phase reactor at room temperature for 16 h. The resin was washed with MeOH (the fraction was discarded) and with NH₃ (7N in MeOH). The filtrates were concentrated in vacuo to give intermediate 4 (1.62 g, 97%) as a pale brown oil.

### Preparation of Intermediate 5

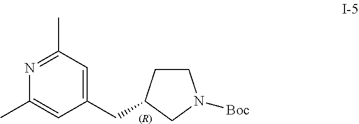

Intermediate 5 was prepared following an analogous procedure to the one reported for the synthesis of intermediate 3, using a 0.32M solution of intermediate 72 in THF and 4-bromo-2,6-dimethylpyridine (CAS: 5093-70-9).

The crude product was purified by flash column chromatography (SiO₂, EtAOc in heptane, gradient from 30/70 to 80/20). The desired fractions were collected and concentrated in vacuo to afford intermediate 5 (2.50 g, 87%) as an oil.

### Preparation of Intermediate 6

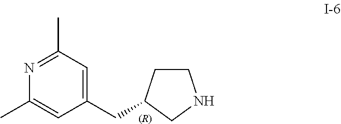

Intermediate 6 was prepared following an analogous procedure to the one reported for the synthesis of intermediate 4 using intermediate 5 as starting material.

The resin was washed with MeOH (the fraction was discarded) and with NH₃ (7N in MeOH). The filtrates were concentrated in vacuo to give intermediate 6 (1.53 g, 93%) as a pale brown oil.

### Preparation of Intermediate 7

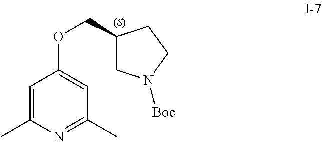

NaH (60% dispersion in mineral oil, 238 mg, 5.96 mmol) was added to a solution of (S)-tert-butyl 3-(hydroxymethyl)pyrrolidine-1-carboxylate (CAS: 199174-24-8; 1.00 g, 4.97 mmol) in DMF (10 mL) at 0° C. under N₂ atmosphere. The reaction mixture was stirred at 0° C. for 30 min, and 4-chloro-2,6-dimethylpyridine (CAS: 3512-75-2; 697 μL, 5.47 mmol) was added dropwise. The reaction mixture was stirred at 0° C. for 1 h and at 80° C. for 20 h. The reaction was quenched with NH₄Cl (sat.) and extracted with EtOAc. The organic layer was dried (MgSO₄), filtered and evaporated in vacuo. The crude product was purified by flash column chromatography (silica; EtOAc in heptane, gradient from 50/50 to 100/0). The desired fractions were collected and concentrated in vacuo to afford intermediate 7 (1.50 g, 99%).

### Preparation of Intermediate 8

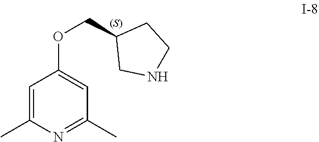

A solution of intermediate 7 (1.50 g, 4.89 mmol) in MeOH (39.8 mL) was added to a closed reactor containing Amberlyst®15 hydrogen form (CAS: 39389-20-3; 5.21 g, 24.5 mmol). The mixture was shaken in a solid phase reactor at room temperature for 16 h. The resin was washed with MeOH (the fraction was discarded) and then with NH₃ (7N in MeOH). The filtrates were concentrated in vacuo to give intermediate 8 (1.00 g, 99%) as a pale brown oil.

### Preparation of Intermediate 9

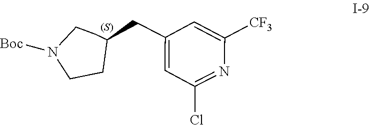

A solution of intermediate 73 in THF (0.15M in THF, 10 mL, 1.50 mmol) was added to a mixture of 2-chloro-4-iodo-6-trifluoromethyl pyridine (CAS: 205444-22-0; 419 mg, 1.36 mmol) and Pd(t-Bu₃P)₂ (34.8 mg, 68.2 μmop under N₂ atmosphere. The reaction mixture was stirred at room temperature for 1 h. The mixture was treated with a 1/1 mixture of NH₄Cl (sat.) and NH₄OH, and extracted with EtOAc. The organic layer was dried (Na₂SO₄), filtered and the solvents were evaporated in vacuo. The crude product was purified by flash column chromatography (silica, EtAOc in heptane, gradient from 0/100 to 20/80). The desired fractions were collected and concentrated in vacuo to yield intermediate 9 (350 mg, 70%) as a pale yellow oil.

### Preparation of Intermediate 10

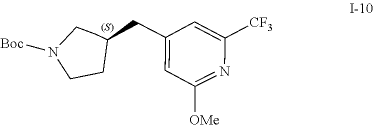

Intermediate 9 (350 mg, 0.96 mmol) was dissolved in an anhydrous solution of NaOMe (0.22 mL, 0.96 mmol, 25% purity) and the mixture was stirred at room temperature for 16 h. Water was added and the aqueous phase was extracted with DCM. The organic layer was dried (Na₂SO₄), filtered and the solvent was evaporated in vacuo to give intermediate 10 (250 mg, 72%) as a colorless oil.

### Preparation of Intermediate 11

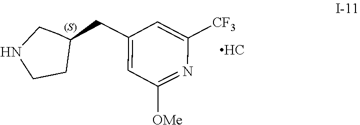

Intermediate 10 (250 mg, 0.69 mmol) was dissolved in HCl (4M in 1,4-dioxane, 0.17 mL, 0.70 mmol) and the reaction mixture was stirred at room temperature for 1 h. The solvent was evaporated in vacuo to afford intermediate 11 as a HCl salt (190 mg, 92%) which was used in the next step without further purification.

### Preparation of Intermediate 12

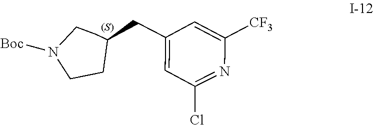

Intermediate 12 was prepare following an analogous procedure to the one described for the synthesis of intermediate 9 using a solution of intermediate 73 in THF and 2-chloro-4-iodo-6-trifluoromethyl pyridine (CAS: 205444-22-0) as starting materials. The crude product was purified by flash column chromatography (silica, EtOAc in heptane, gradient from 0/100 to 20/80). The desired fractions were collected and concentrated in vacuo to afford intermediate 12 (2.50 g, 40%, 75% purity) as a pale yellow oil.

### Preparation of Intermediate 13

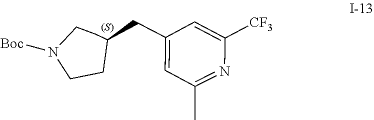

K₂CO₃ (1.42 g, 10.3 mmol) was added to a stirred solution of intermediate 12 (2.50 g, 5.14 mmol, 75% purity) in 1,4-dioxane (15 mL). The mixture was deoxygenated with a N₂ flow for 5 min. Trimethylboroxine (CAS: 823-96-1; 1.29 mL, 9.25 mmol), Pd(OAc)₂ (57.7 mg, 0.26 mmol) and tricyclohexylphosphine tetrafluoroborate (CAS: 58656-04-5; 189 mg, 0.51 mmol) were added. The reaction mixture was stirred at 100° C. for 2 h under N₂. The mixture was cooled down, washed with H₂O and extracted with DCM. The organic layer was dried (MgSO₄), filtered and the solvents were evaporated in vacuo. The crude product was purified by flash column chromatography (silica, EtOAc in heptane, gradient from 0/100 to 15/85). The desired fractions were collected and concentrated in vacuo to yield intermediate 13 (1.90 g, 86%, 80% purity) as a grey oil.

### Preparation of Intermediate 14

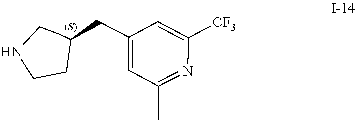

Amberlyst®15 hydrogen form (CAS: 39389-20-3; 4.40 g) was added to a stirred solution of intermediate 13 (1.90 g, 4.14 mmol, 80% purity) in MeOH (22.4 mL). The reaction mixture was shaken in a solid phase reactor at room temperature for 16 h. The resin was washed with MeOH (the fraction was discarded) and with NH₃ (7N in MeOH). The filtrate was concentrated in vacuo to give intermediate 14 (980 mg, 91%) as a brown oil.

### Preparation of Intermediate 15

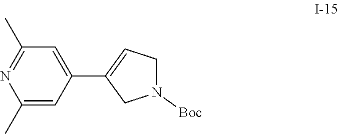 [SMILES: Cc1cc(C2=CCN(C(=O)OC(C)(C)C)C2)cc(C)n1]

1,4-dioxane (3.57 mL) and Na₂CO₃ (sat., aq., 2.5 mL) were added to a stirred mixture of 4-chloro-2,6-dimethylpyridine (CAS: 3512-75-2; 0.20 g, 1.41 mmol), tert-butyl 3-(tetramethyl-1,3,2-dioxaborolan-2-yl)-2,5-dihydro-1H-pyrrole-1-carboxylate (CAS: 212127-83-8; 0.48 g, 1.63 mmol) and Pd(PPh₃)₄ (167 mg, 0.15 mmol) in a sealed tube and under N₂ atmosphere. The reaction mixture was stirred at 130° C. for 30 min. The mixture was treated with water and extracted with DCM. The organic layer was dried (Na₂SO₄), filtered and the solvents were evaporated in vacuo. The crude product was purified by flash column chromatography (silica, EtOAc in heptane, gradient from 0/100 to 100/0). The desired fractions were collected and concentrated in vacuo to give intermediate 15 (207 mg, 53%) as a pale yellow oil that solidified upon standing.

### Preparation of Intermediate 16

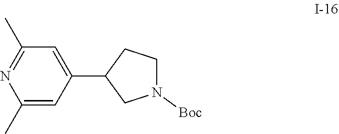 [SMILES: Cc1cc(C2CCN(C(=O)OC(C)(C)C)C2)cc(C)n1]

A mixture of intermediate 15 (200 mg, 0.73 mmol) and Pd/C (10%) in EtOH (14 mL) was hydrogenated in a H-cube. The solvent was evaporated in vacuo to afford intermediate 16 (190 mg, 94%) as a colorless oil.

### Preparation of Intermediate 17

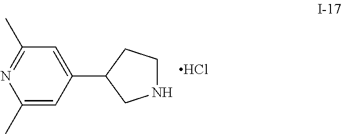

Intermediate 16 (190 mg, 0.69 mmol) was dissolved in HCl (4M in 1,4-dioxane, 1.0 mL, 4.0 mmol) and the reaction mixture was stirred at room temperature for 1 h. The solvent was evaporated in vacuo to afford intermediate 17 as a HCl salt (145 mg, quant.) which was used in the next step without further purification.

### Preparation of Intermediate 20

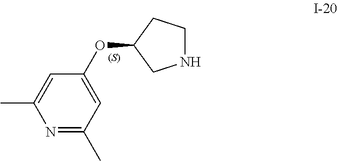

Amberlyst®15 hydrogen form (CAS: 39389-20-3, 6.01 g, loading 4.7 meq/g) was added to a stirred solution of intermediate 44 (1.67 g, 5.71 mmol) in MeOH (44 mL) at rt. The mixture was shaken in a solid phase reactor at rt for 16 h. The resin was washed with MeOH (this fraction was discarded). Then NH₃ (7N solution in MeOH, 31.7 mL) was added. The mixture was shaken in the solid phase reactor for 2 h. The resin was filtered off and was washed twice with additional NH₃ (7N solution in MeOH, 2×31 mL; 30 min shaken). The filtrates were concentrated in vacuo to yield Intermediate 20 (960 mg, 87%) as a brown oil.

### Preparation of Intermediate 21

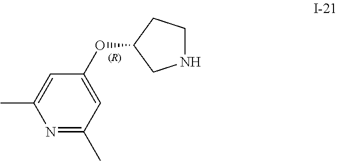

Intermediate 21 was prepared following an analogous procedure to the one described for the synthesis of intermediate 20 using intermediate 45 as starting material.

### Preparation of Intermediate 22

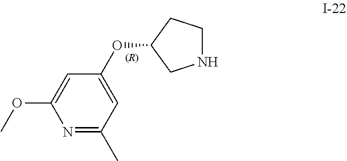

Intermediate 22 was prepared following an analogous procedure to the one described for the synthesis of intermediate 20 using intermediate 46 as starting material.

### Preparation of Intermediate 23

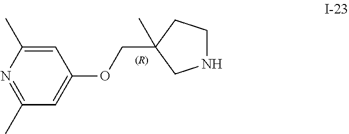

HCl (9.55 mL, 38.2 mmol, 4 M solution in 1,4-dioxane) was added to intermediate 47 at rt. The mixture was further stirred at rt for 90 min. The volatiles were evaporated in vacuo and the residue thus obtained was dissolved in MeOH and passed through an Isolute SCX-2 cartridge and the product was eluted with 7N ammonia in methanol to yield intermediate 23 (336 mg, 95%) as a brownish oil.

### Preparation of Intermediate 24

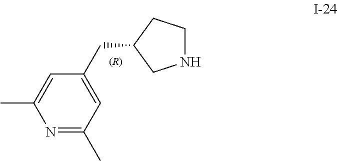

Intermediate 24 was prepared following an analogous procedure to the one described for the synthesis of intermediate 20 using intermediate 48 as starting material.

### Preparation of Intermediate 25

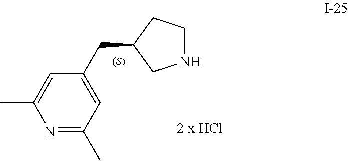

HCl (47.98 mL, 287.91 mmol, 6M in isopropanol) was added to a solution of intermediate 49 (8.36 g, 28.8 mmol) in MeOH (70 mL) at rt. The mixture was further stirred at 50° C. for 1 h. The volatiles were evaporated under vacuum affording crude intermediate 25 (7.35 g, 97%. 2×HCl salt) as white solid.

### Preparation of Intermediate 26

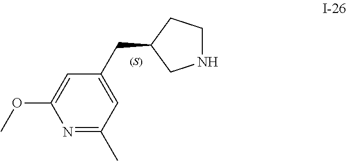

Intermediate 26 was prepared following an analogous procedure to the one described for the synthesis of intermediate 20 using intermediate 50 as starting material.

### Preparation of Intermediate 27

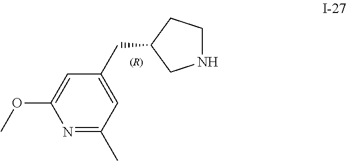

Intermediate 27 was prepared following an analogous procedure to the one described for the synthesis of intermediate 20 using intermediate 51 as starting material.

### Preparation of Intermediate 28

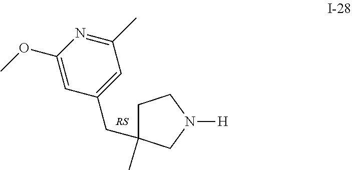 [SMILES: COc1cc(CC2(C)CCNC2)cc(C)n1]

Intermediate 28 was prepared following an analogous procedure to the one described for the synthesis of intermediate 23 using intermediate 52 as starting material.

### Preparation of Intermediate 29

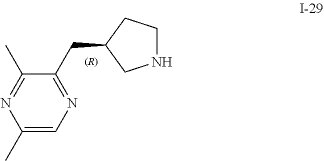

Intermediate 29 was prepared following an analogous procedure to the one described for the synthesis of intermediate 20 using intermediate 53 as starting material.

### Preparation of Intermediate 30

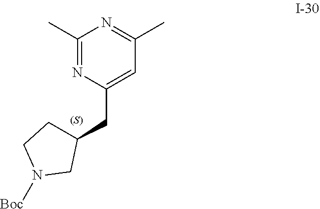

A solution of intermediate 73 (0.33 Min THF, 47.4 mL, 15.7 mmol) was added to a mixture of 4-chloro-2,6-dimethylpyrimidine (CAS: 4472-45-1; 2.03 g, 14.2 mmol) and Pd(tBu₃P)₂ (0.60 g, 0.85 mmol) under N₂ atmosphere. TMEDA (CAS: 110-18-9; 2.33 mL, 15.7 mmol) was added and the reaction mixture was stirred at 60° C. for 18 h. The reaction was quenched with a 1/1 solution of NH₄Cl (sat.) and NH₃ (32% aq.). The mixture was extracted with EtOAc. The organic layer was dried (Na₂SO₄), filtered and the solvents were evaporated in vacuo. The crude product was purified by flash column chromatography (SiO₂, EtOAc in heptane, gradient from 0/100 to 80/20). The desired fractions were collected and the solvents were evaporated in vacuo to yield intermediate 30 (735 mg, 9%, 51% purity) as yellow oil.

### Preparation of Intermediate 31

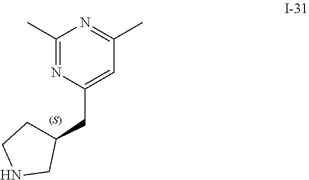

A solution of intermediate 30 (735 mg, 2.52 mmol, 51% purity) in MeOH (19.4 mL) was added to a closed reactor containing Amberlyst®15 hydrogen form (CAS: 39389-20-3; 2.68 g, 12.6 mmol). The mixture was shaken in a solid phase reactor at room temperature for 16 h. The resin was washed with MeOH (the fraction was discarded). NH₃ (7N in MeOH) (25 mL) was added. The mixture was shaken in the solid phase reactor for 2 h. The resin was filtered off and washed with NH₃ (7N in MeOH) (2×25 mL; 30 min shaken). The filtrates were concentrated in vacuo to afford intermediate 31 (430 mg, 89%) as a light brown oil.

### Preparation of Intermediate 32

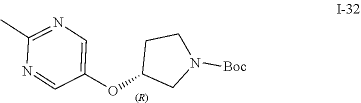

DBAD (CAS: 870-50-8; 1.36 g, 5.90 mmol) was added to a mixture of 2-methylpyrimidin-5-ol (CAS: 35231-56-2; 500 mg, 4.54 mmol), (R)-(−)-N-boc-3-pyrrolidinol (CAS: 109431-87-0; 1.11 g, 5.90 mmol) and triphenylphosphine (1.55 g, 5.90 mmol) in THF (10 mL). The reaction mixture was stirred at room temperature for 18 h and concentrated to dryness. The residue was purified by flash column chromatography (silica, EtOAc in heptane, gradient from 0/100 to 100/0). The desired fractions were collected and concentrated in vacuo to yield intermediate 32 (1.1 g, 87%) as a colorless oil.

### Preparation of Intermediate 33

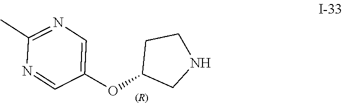

HCl (4M in 1,4-dioxane, 10 mL, 40 mmol) was added to intermediate 32 (1.10 g, 3.94 mmol) and the reaction mixture was stirred at room temperature for 3 h. The reaction mixture was concentrated to dryness. The residue was suspended in EtOAc and basified with NH₄OH. The aqueous layer was extracted with EtOAc. The combined organic layers were dried (MgSO4), filtered and the solvent was evaporated in vacuo to afford intermediate 32 (560 mg, 79%) as a white solid.

### Preparation of Intermediate 34

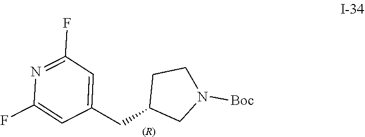

A 0.38M solution of intermediate 72 (11 mL, 4.18 mmol), TMEDA (CAS: 110-18-9; 0.63 mL, 4.20 mmol) and Pd(PPh₃)₂Cl₂ (68.0 mg, 96.6 μmop were successively added to a 2-bromo-3,5-difluoropyridine (CAS:660425-16-1; 761 mg, 3.92 mmol) in a sealed tube and under N₂ atmosphere. The reaction mixture was stirred at 65° C. for 16 h. The reaction was quenched with a 1/1 solution of NH₄Cl (sat.) and NH₃ (26%, aq.) and extracted with EtAOc. The organic layer was dried (MgSO₄), filtered and the solvent was evaporated in vacuo. The crude product was purified by flash column chromatography (SiO₂, EtOAc in heptane, gradient from 0/100 to 30/70). The desired fractions were collected and concentrated in vacuo to afford intermediate 34 (715 mg, 61%) as a yellow oil

### Preparation of Intermediate 35

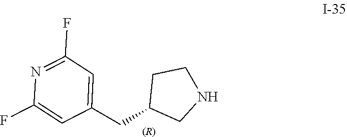

A solution of intermediate 34 (1.16 g, 3.91 mmol) in MeOH (19.7 mL) was added dropwise to Amberlyst®15 hydrogen form (CAS: 39389-20-3; 3.93 g, 18.5 mmol) in a solid phase reactor. Once the evolution of CO₂ stopped, the mixture was shaken at room temperature for 2 days. The resin was washed with MeOH (the fraction was discarded) and with NH₃ (7N in MeOH). The filtrate was concentrated in vacuo to give intermediate 35 (698 mg, 90%) as a brown oil.

### Preparation of Intermediate 36

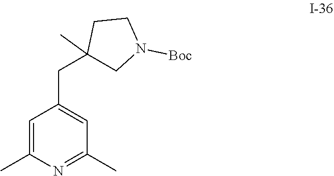 [SMILES: Cc1cc(CC2(C)CCN(C(=O)OC(C)(C)C)C2)cc(C)n1]

Intermediate 74 (0.24M in THF, 11.0 mL, 2.64 mmol) was added to a flask containing 4-bromo-2,6-dimethylpyridine (CAS: 5093-70-9; 492 mg, 2.64 mmol) and Pd(PPh₃)₂Cl₂ (111 mg, 0.16 mmol) under N₂ atmosphere. TMEDA (CAS: 110-18-9; 0.43 mL, 2.91 mmol) was added and the reaction mixture was stirred at 60° C. for 2.5 h. The reaction was quenched by the addition of a 1/1 solution of NH₄Cl (sat.) and NH₃ (32%, aq.). The mixture was extracted with EtOAc. The combined organic layers were dried (Na₂SO₄), filtered and the solvents were evaporated in vacuo. The crude product was purified by flash column chromatography (SiO₂, EtOAc in heptane, gradient from 0/100 to 80/20). The desired fractions were collected and the solvents were evaporated in vacuo to afford intermediate 36 (578 mg, 72%) as a yellow oil.

### Preparation of Intermediate 37

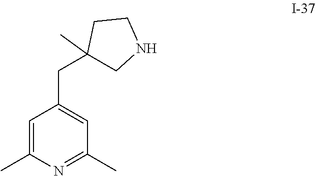

HCl (4M in 1,4-dioxane, 11.4 mL, 45.4 mmol) was added to intermediate 36 (578 mg, 1.90 mmol) at room temperature and the reaction mixture was stirred for 12 h. The volatiles were evaporated in vacuo. The residue was dissolved in MeOH and passed through an Isolute SCX-2 cartridge. The MeOH output solution was discarded. The product was eluted with NH₃ (7N in MeOH) to give intermediate 37 (384 mg, 99%) as a colorless oil.

### Preparation of Intermediate 38

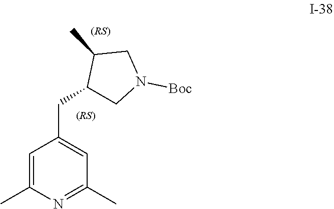 [SMILES: Cc1cc(C[C@H]2CN(C(=O)OC(C)(C)C)C[C@@H]2C)cc(C)n1]

Intermediate 38 was prepared following an analogous procedure to the one described for the synthesis of intermediate 36 using intermediate 133 (0.29 M in THF) and 4-bromo-2,6-dimethylpyridine (CAS: 5093-70-9) as starting materials.

The crude product was purified by flash column chromatography (SiO₂, EtOAc in heptane, gradient from 0/100 to 70/30). The desired fractions were collected and the solvents were evaporated in vacuo to afford intermediate 38 (1.17 g, 65%) as a yellow oil.

### Preparation of Intermediate 39

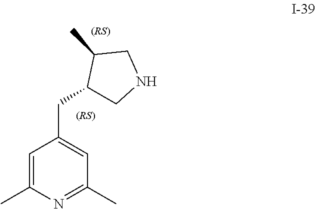

Intermediate 39 was prepared following an analogous procedure to the one described for the synthesis of intermediate 37 using intermediate 38 as starting material.

The residue was dissolved in MeOH and passed through an Isolute SCX-2 cartridge. The MeOH output solution was discarded and the product was eluted with NH₃ (7N in MeOH) to afford intermediate 39 (645 mg, 82%) as a pale yellow oil.

### Preparation of Intermediate 40

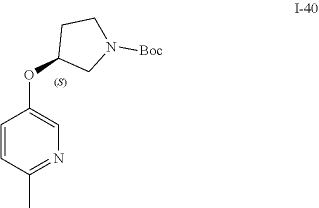

DBAD (CAS: 870-50-8; 37.0 mg, 0.16 mmol) was added dropwise to a mixture of (R)-(−)-N-Boc-3-pyrrolidinol (CAS: 109431-87-0, 20.0 mg, 0.11 mmol), 5-hydroxy-2-methylpyridine (CAS: 1121-78-4; 11.7 mg, 0.11 mmol) and triphenylphosphine (42.0 mg, 0.16 mmol) in toluene (0.57 mL) at 0° C. while the solution was bubbled with N₂. The reaction mixture was stirred overnight at 60° C. and concentrated in vacuo. The crude mixture was used as such in the next step.

### Preparation of Intermediate 41

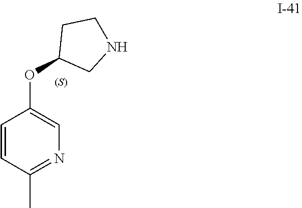

HCl (4M in 1,4-dioxane, 3.34 mL, 13.3 mmol) was added to intermediate 40 (crude, 372 mg) and the reaction mixture was stirred at room temperature for 3 h. The residue was purified by ion exchange chromatography (ISOLUTE SCX2 cartridge) eluting with MeOH, then with 7M solution of NH₃ in MeOH. The desired fractions were collected and concentrated in vacuo to afford intermediate 41 (63.9 mg) as a colorless solid.

### Preparation of Intermediate 42

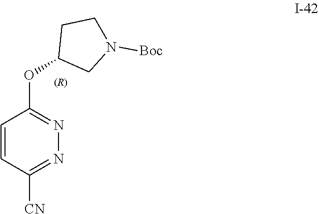

A solution of (R)-(−)-N-boc-3-pyrrolidinol (CAS: 109431-87-0; 0.80 g, 4.27 mmol) in DMF (3.31 mL) was added dropwise to a stirred mixture of NaH (60% dispersion in mineral oil, 0.21 g, 5.13 mmol) and 15-crown-5 (1.14 mL, 4.27 mmol) in DMF (3.31 mL) at 0° C. The reaction mixture was stirred at 0° C. for 30 min and a solution of 6-chloro-3-pyridazinecarbonitrile (CAS: 35857-89-7; 656 mg, 4.70 mmol) dissolved in DMF (3.1 mL) was added portionwise at 0° C. The resulting mixture was stirred at 80° C. for 3 h. The mixture was concentrated in vacuo and the residue was diluted with water and extracted with EtOAc. The organic layer was dried (MgSO₄), filtered and evaporated in vacuo. The crude product was purified by flash column chromatography (silica, EtOAC in heptane, gradient from 0/100 to 60/40). The desired fractions were collected and concentrated in vacuo to afford intermediate 42 (810 mg, 65%) as a white solid.

### Preparation of Intermediate 43

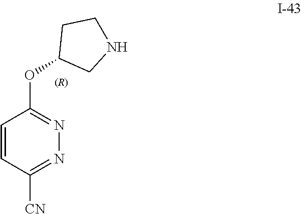

HCl (4M in 1,4-dioxane, 6.98 mL, 27.9 mmol) was added to intermediate 42 (810 mg, 2.79 mmol) and the reaction mixture was stirred at room temperature for 3 h. The reaction mixture was concentrated to dryness. The residue was suspended in EtOAc and basified with NH₄Cl. The aqueous phase was extracted with EtOAc. the combined organic extracts were dried (MgSO₄), filtered and the solvent was evaporated in vacuo to afford intermediate 43 which was used as such in the next step.

### Preparation of Intermediate 44

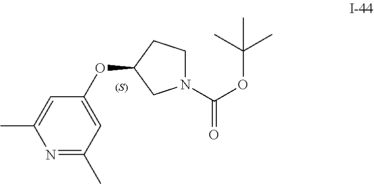

Sodium hydride (341.8 mg, 8.55 mmol) was added to a stirred solution of (3S)-1-Boc-3-hydroxypyrrolidine (CAS: 109431-87-0, 1600 mg, 8.55 mmol) in DMF (4.12 mL) at 0° C. and the mixture was stirred for 30 min. Then the mixture was allowed to warm to rt and a solution of 4-chloro-2,6-dimethylpyridine (CAS: 3512-75-2, 1.09 mL, 8.55 mmol) in DMF (2.78 mL) was added dropwise. The mixture was stirred at rt for 16 h and then at 60° C. for 6 h. After cooling to rt, water was added and the mixture was extracted with EtOAc. The organic layer was dried over Na₂SO₄, filtered and concentrated in vacuo. The residue was purified by flash chromatography (silica gel, EtOAc in heptane: 0/100 to 30/70). The desired fractions were collected and concentrated in vacuo to yield intermediate 44 (1670 mg, 67%) as a colorless oil.

### Preparation of Intermediate 45

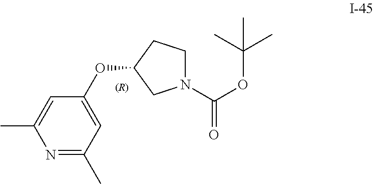

Intermediate 45 was prepared following an analogous procedure to the one described for the synthesis of intermediate 44 using (3R)-1-Boc-3-hydroxypyrrolidine (CAS: 109431-87-0) and 4-chloro-2,6-dimethylpyridine (CAS: 3512-75-2) as starting materials.

### Preparation of Intermediate 46

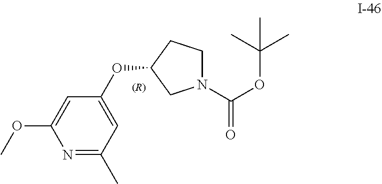

Intermediate 46 was prepared following an analogous procedure to the one described for the synthesis of intermediate 44 using (3R)-1-Boc-3-hydroxypyrrolidine (CAS: 109431-87-0) and 4-bromo-2-methoxy-6-methylpyridine (CAS: 1083169-00-9) as starting materials.

### Preparation of Intermediate 47

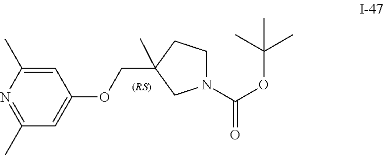 [SMILES: Cc1cc(OCC2(C)CCN(C(=O)OC(C)(C)C)C2)cc(C)n1]

Intermediate 47 was prepared following an analogous procedure to the one described for the synthesis of intermediate 44 using 1-Boc-3-methylpyrrolidine-3-methanol (CAS: 1263506-20-2) and 4-chloro-2,6-dimethylpyridine (CAS: 3512-75-2) as starting materials.

### Preparation of Intermediate 48

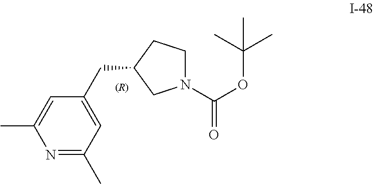

A solution of intermediate 72 (0.24 M in THF, 43 mL, 10.32 mmol) was added to a flask containing 4-bromo-2,6-dimethylpyridine (CAS: 5093-70-9, 1.75 g, 9.38 mmol) and bis(triphenylphosphine)palladium(II) dichloride (0.395 g, 0.56 mmol) under N₂. Then N,N,N′,N′-tetramethylethylenediamine (1.538 mL, 10.32 mmol) was added and the mixture was stirred at 60° C. for 18 h. The mixture was quenched with the addition of a 1/1 solution of sat NH₄Cl/32% aq NH₃ and then it was extracted with EtOAc. The organic layer was separated, dried (Na₂SO₄), filtered and the solvents evaporated in vacuo. The crude product was purified by flash column chromatography (SiO₂, EtOAc in heptane 0/100 to 80/20). The desired fractions were collected and the solvents evaporated in vacuo to yield intermediate 48 (2.59 g, 955) as a yellow oil.

### Preparation of Intermediate 49

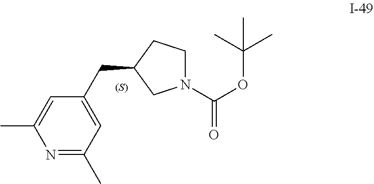

Intermediate 49 was prepared following an analogous procedure to the one described for the synthesis of intermediate 48 using intermediate 73 and 4-bromo-2,6-dimethylpyridine (CAS: 5093-70-9) as starting materials.

### Preparation of Intermediate 50

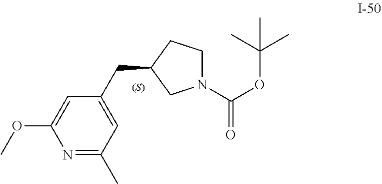

Intermediate 50 was prepared following an analogous procedure to the one described for the synthesis of intermediate 48 using intermediate 73 and 4-bromo-2-methoxy-6-methylpyridine (CAS: 1083169-00-9) as starting materials.

### Preparation of Intermediate 51

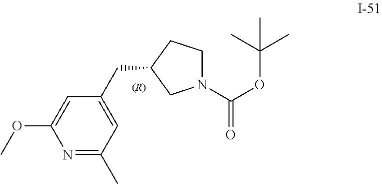

Intermediate 50 was prepared following an analogous procedure to the one described for the synthesis of intermediate 48 using intermediate 72 and 4-bromo-2-methoxy-6-methylpyridine (CAS: 1083169-00-9) as starting materials.

### Preparation of Intermediate 52

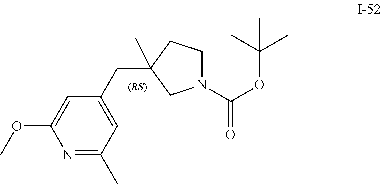 [SMILES: COc1cc(CC2(C)CCN(C(=O)OC(C)(C)C)C2)cc(C)n1]

Intermediate 52 was prepared following an analogous procedure to the one described for the synthesis of intermediate 48 using intermediate 74 and 4-bromo-2-methoxy-6-methylpyridine (CAS: 1083169-00-9) as starting materials.

### Preparation of Intermediate 53

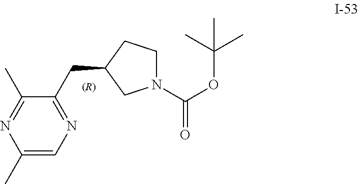

Intermediate 53 was prepared following an analogous procedure to the one described for the synthesis of intermediate 48 using intermediate 73 and 2-chloro-3,5-dimethylpyrazine (CAS: 38557-72-1) as starting materials.

### Preparation of Intermediate 54

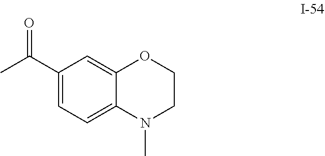

n-Butyl lithium (1.6M in hexane, 6.85 mL, 11.0 mmol) was added dropwise to a stirred solution of 7-bromo-4-methyl-3,4-dihydro-2H-1,4-benzoxazine (CAS: 154264-95-6; 2.00 g, 8.77 mmol) in Me-THF (30 mL) under N₂ atmosphere at −78° C. The reaction mixture was stirred at −78° C. for 30 min and a solution of N-methoxy-N-methylacetamide (CAS:78191-00-1; 1.81 g, 17.5 mmol) in Me-THF (10 mL) was added dropwise. The reaction mixture was stirred at −78° C. for 1 h and at room temperature for 1 h. The reaction was quenched with NH₄Cl (sat.) and extracted with EtOAc. The organic layer was dried (Na₂SO₄), filtered and concentrated in vacuo. The residue was purified by flash column chromatography (silica, EtOAc in heptane, gradient from 0/100 to 30/70). The desired fractions were collected and concentrated in vacuo to afford intermediate 54 (818 mg, 49%) as a yellow oil.

### Preparation of Intermediate 55

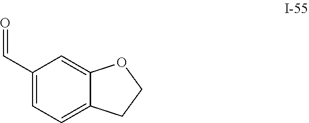 [SMILES: O=Cc1ccc2c(c1)OCC2]

n-Butyl lithium (1.6M in hexane, 3.87 mL, 6.18 mmol) was added dropwise to a stirred solution of 6-bromo-2,3-dihydrobenzofuran (CAS: 189035-22-1; 1.00 g, 5.02 mmol) in Me-THF (24.1 mL) under N₂ atmosphere at −78° C. The reaction mixture was stirred at −78° C. for 30 min and DMF (0.97 mL, 12.5 mmol) was added dropwise. The reaction mixture was stirred at −78° C. for 2 h, quenched with NH₄Cl (sat.) and extracted with EtOAc. The organic layer was dried (MgSO₄), filtered and concentrated in vacuo. The crude product was purified by flash column chromatography (SiO₂, EtOAc in heptane, gradient from 0/100 to 100/0). The desired fractions were collected and concentrated in vacuo to afford intermediate 55 (631 mg, 85%) as a cream solid.

### Preparation of Intermediate 56

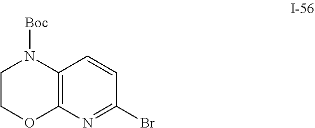 [SMILES: CC(C)(C)OC(=O)N1CCOc2nc(Br)ccc21]

Borane dimethyl sulfide complex (CAS: 13292-87-0; 950 μL, 10.0 mmol) was added dropwise to a stirred suspension of 6-bromo-1H-pyrido[2,3-b][1,4]oxazin-2(3H)-one (CAS: 1245708-13-7; 1.13 g, 4.95 mmol) in THF (26 mL) under N₂ atmosphere. The reaction mixture was stirred under reflux for 2 h. The mixture was cooled to 0° C. and MeOH (10 mL) was added dropwise. The mixture was stirred at room temperature for 1 h. The solvent was evaporated in vacuo. The crude mixture was dissolved with THF (26 mL) and cooled to 0° C. Boc anhydride (CAS: 24424-99-5; 1.2 mL, 1.1 eq) and lithium bis(trimethylsilyl)amide (1M in THF, 5.75 mL) were added dropwise. The reaction mixture was stirred at 0° C. for 2 h and at room temperature for 1 h. The mixture was cooled to 0° C. and Boc anhydride (0.25 mL, 0.2 eq) and lithium bis(trimethylsilyl)amide (1M in THF, 1 mL) were added dropwise. The reaction mixture was stirred at 0° C. for 1 h and treated with NH₄Cl (sat.). The mixture was extracted with EtOAc. The organic layer was dried (MgSO₄), filtered and the solvents were evaporated in vacuo. The crude product was purified by flash column chromatography (SiO₂, EtOAc in heptane, gradient from 0/100 to 70/30). The desired fractions were collected and concentrated in vacuo to give intermediate 56 (1.29 g, 83%) as a pale yellow oil that precipitated upon standing.

### Preparation of Intermediate 57

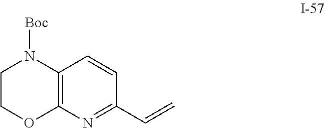 [SMILES: C=Cc1ccc2c(n1)OCCN2C(=O)OC(C)(C)C]

K₂CO₃ (sat., aq., 22 mL), vinylboronic acid pinacol ester (CAS: 75927-49-0; 0.95 mL, 5.60 mL) and Pd(PPh₃)₄ were successively added to a stirred solution of intermediate 56 (1.28 g, 4.06 mmol) in 1,4-dioxane (41 mL) under N₂ atmosphere. The reaction mixture was stirred at 80° C. for 20 h. The mixture was treated with water and extracted with EtAOc. The organic layer was dried (MgSO₄), filtered and the solvents were evaporated in vacuo. The crude product was purified by flash column chromatography (SiO₂, EtOAc in heptane, gradient from 0/100 to 50/50). The desired fractions were collected and concentrated in vacuo to yield intermediate 57 (940 mg, 88%) as a yellow oil.

### Preparation of Intermediate 58

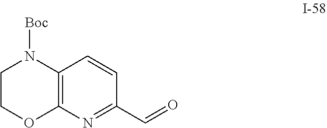 [SMILES: CC(C)(C)OC(=O)N1CCOc2nc(C=O)ccc21]

Sodium periodate (CAS: 7790-28-5; 1.71 g, 8.01 mmol) and osmium tetroxide (2.5% in t-BuOH, 066 mL, 48.8 μmop were added to a stirred solution of intermediate 57 (940 mg, 3.58 mmol) in 1,4-dioxane (27.7 mL) and H₂O (11.1 mL) under N₂ atmosphere. The reaction mixture was stirred at room temperature for 18 h. The mixture was treated with Na₂S₂O₃ (sat.) and extracted with EtOAc. The organic layer was dried (MgSO₄), filtered and the solvents were evaporated in vacuo. The crude product was purified by flash column chromatography (SiO₂, EtOAc in heptane, gradient from 0/100 to 100/0). The desired fractions were collected and concentrated in vacuo to yield intermediate 58 (440 mg, 46%) as a white solid.

### Preparation of Intermediate 59

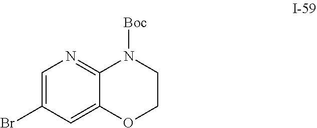 [SMILES: CC(C)(C)OC(=O)N1CCOc2cc(Br)cnc21]

Lithium bis(trimethylsilyl)amide (1M in THF, 1.1 equiv.) was added dropwise over 10 min to a stirred mixture of 7-bromo-3,4-dihydro-2H-pyrido[3,2-b][1,4]oxazine (CAS: 34950-82-8; 3.00 g, 14.0 mmol) and boc-anhydride (CAS: 24424-99-5; 1.1 equiv.) in THF (67.8 mL) at 0° C. and under N₂ atmosphere. The reaction mixture was stirred at 0° C. for 2 h and additional quantity of boc-anhydride (0.52 equiv.) in THF (10 mL) was added at 0° C. The reaction mixture was stirred at 0° C. for 1 h, treated with NH₄Cl (sat.) and extracted with EtOAc. The organic layer was dried (Na₂SO₄), filtered and the solvents were evaporated in vacuo. The crude product was purified by flash column chromatography (SiO₂, EtOAc in DCM, gradient from 0/100 to 2/98). The desired fractions were collected and concentrated in vacuo to afford intermediate 59 (3.66 g, 83%) as a beige solid.

### Preparation of Intermediate 60

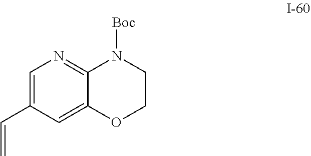 [SMILES: C=Cc1cnc2c(c1)OCCN2C(=O)OC(C)(C)C]

Pd(PPh₃)₄ (0.67 g, 0.58 mmol) followed by vinylboronic acid pinacol ester (CAS: 75927-49-0; 2.46 mL, 14.5 mmol) were added to a deoxygenated solution of intermediate 59 (3.66 g, 11.6 mmol) in K₂CO₃ (sat. aq., 29 mL) and 1,4-dioxane (57.9 mL) under N₂ atmosphere. The reaction mixture was stirred at 80° C. for 18 h. The mixture was treated with water and extracted with EtOAc. The organic layer was dried (Na₂SO₄), filtered and the solvents were evaporated in vacuo. The crude product was purified by flash column chromatography (SiO₂, EtOAc in DCM, gradient from 0/100 to 5/95). The desired fractions were collected and concentrated in vacuo to afford intermediate 60 (2.69 g, 88%) as a brown solid.

### Preparation of Intermediate 61

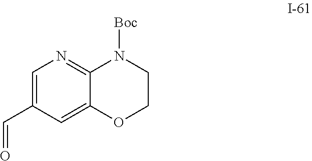 [SMILES: CC(C)(C)OC(=O)N1CCOc2cc(C=O)cnc21]

Sodium periodate (CAS: 7790-28-5; 4.9 g, 22.9 mmol) followed by osmium tetroxide (2.5% in t-BuOH, 1.89 mL, 0.14 mmol) were added to a stirred mixture of intermediate 60 (2.69 g, 10.2 mmol) in 1,4-dioxane (79.3 mL) and H₂O (31.7 mL) under N₂ atmosphere. The reaction mixture was stirred at room temperature for 4.5 h, treated with Na₂S₂O₃ (sat.) and extracted with EtOAc. The organic layer was dried (Na₂SO₄), filtered and the solvents were evaporated in vacuo. The crude product was purified by flash column chromatography (SiO₂, EtOAc in heptane, gradient from 0/100 to 40/60). The desired fractions were collected and concentrated in vacuo to afford intermediate 61 (1.93 g, 71%) as a white solid.

### Preparation of Intermediate 62

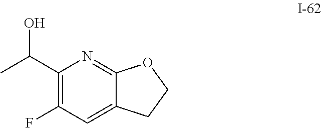

NaBH₄ (55.5 mg, 1.47 mmol) was added to a solution of intermediate 97 (133 mg, 0.73 mmol) in EtOH (3 mL) at 0° C. The reaction mixture was stirred at room temperature for 30 min and the reaction was quenched with NH₄Cl (sat., aq.). The mixture was extracted with DCM. The combined organic layers were dried (MgSO₄), filtered and concentrated in vacuo to afford intermediate 62 (130 mg, 97%) as an oil.

### Preparation of Intermediate 63

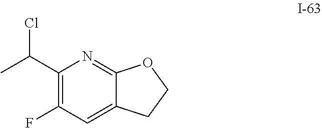

Thionyl chloride (0.8 mL, 11.0 mmol) was added to a solution of intermediate 62 (500 mg, 2.73 mmol) in DCM (12 mL) at 0° C. The reaction mixture was stirred at room temperature for 2 h, diluted with water and extracted with DCM. The combined organic layers were dried (MgSO₄), filtered and evaporated in vacuo to yield intermediate 63 (520 mg) which was used in the next step without any purification.

### Preparation of Intermediate 64

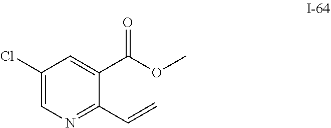

A mixture of methyl 2,5-dichloronicotinate (CAS: 67754-03-4; 1.95 g, 9.47 mmol), vinylboronic acid pinacol ester (CAS: 75927-49-0; 1.79 mL, 10.4 mmol), Pd(PPh₃)₄ (547 mg, 0.47 mmol) and K₂CO₃ (2M, 9.47 mL, 18.9 mmol) in 1,2-dimethoxyethane (24.6 mL) was stirred under N₂ atmosphere for 3 h at 120° C. The suspension was filtered through Celite® and evaporated in vacuo. The residue taken up in water and extracted with DCM. The organic layer was dried (NaSO₄), filtered and evaporated in vacuo. The residue was purified by flash column chromatography (silica, DCM). The desired fractions were collected and concentrated in vacuo to afford intermediate 64 (1.46 g, 78%) as a brown oil.

### Preparation of Intermediate 65

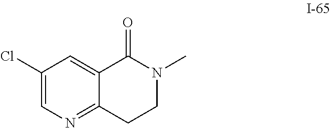

A mixture of intermediate 64 (1.40 g, 4.68 mmol) and methylamine hydrochloride (CAS: 593-51-1; 473 mg, 7.01 mmol) in DIPEA (2M in NMP, 5.85 mL, 11.7 mmol) was stirred at 160° C. for 10 min under microwave irradiation. The mixture was diluted with EtOAc and washed with NaHCO₃ (sat.) and brine. The organic layer was dried (Na₂SO₄), filtered and concentrated in vacuo. The residue was purified by flash column chromatography (silica, EtOAc in DCM, gradient from 0/100 to 30/70). The desired fractions were collected and concentrated in vacuo to afford intermediate 65 (633 mg, 69%) as a red oil, that solidified upon standing.

### Preparation of Intermediate 66

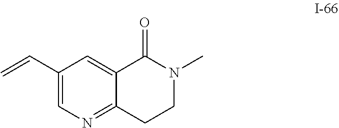

Pd(dtbpf)Cl₂ (167 mg, 0.25 mmol) was added to a mixture of intermediate 65 (500 mg, 2.54 mmol), potassium trifluoro(vinyl)borate (CAS: 13682-77-4; 681 mg, 5.09 mmol) and K₃PO₄ (1.62 g, 7.63 mmol) in 1,4-dioxane (11.9 mL) and H₂O (4.13 mL) under N₂ atmosphere. The reaction mixture was stirred at 110° C. for 16 h. The suspension was filtered through Celite® and washed with water and EtOAc. The organic layer was separated, dried (Na₂SO), filtered and evaporated in vacuo. The crude mixture was purified by flash column chromatography (silica, EtOAc in DCM, gradient from 0/100 to 20/80). The desired fractions were collected and concentrated in vacuo to afford intermediate 66 (569 mg, 89% purity) which was used as such in the next step.

### Preparation of Intermediate 67

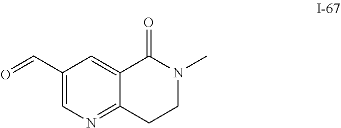

Osmium tetroxide (2.5% in t-BuOH, 0.53 mL, 38.9 μmol) followed by sodium periodate (1.39 g, 6.52 mmol) were added to a stirred solution of intermediate 66 (569 mg, 3.02 mmol, 89% purity) in 1,4-dioxane (22.1 mL) and H₂O (9.47 mL) under N₂ atmosphere. The reaction mixture was stirred at room temperature for 1 h, treated with Na₂S₂O₃ (sat.) and extracted with EtOAc. The organic layer was dried (MgSO₄), filtered and the solvents were evaporated in vacuo. The crude product was purified by flash column chromatography (SiO₂, EtOAc in DCM, gradient from 0/100 to 50/50). The desired fractions were collected and concentrated in vacuo to afford intermediate 67 (217 mg, 38%) as a yellow wax.

### Preparation of Intermediate 72

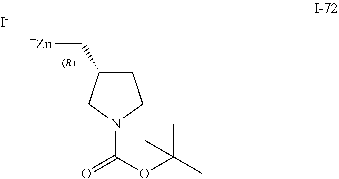

A solution of (3R)-1-Boc-3-iodomethylpyrrolidine (CAS: 1187932-69-9; 10.1 g, 32.4 mmol) in THF (65 mL) was pumped through a column containing activated Zn (30 g, 458.8 mmol) at 40° C. with flow of 1 mL/min. The outcome solution was collected under N₂ atmosphere to yield intermediate 72 as a clear solution that was used without any further manipulation.

For the above reaction Zn was activated as follows: A solution of TMSCl (2 mL) and 1-bromo-2-chloroethane (1.2 mL) in THF (20 mL) was passed through the column containing Zn at a flow of 1 mL/min.

### Preparation of Intermediate 73

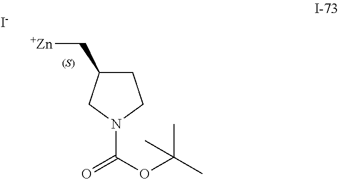

Intermediate 73 was prepared following an analogous procedure to the one described for the synthesis of intermediate 72 using (3S)-1-Boc-3-iodomethylpyrrolidine (CAS: 224168-68-7 as starting material.

### Preparation of Intermediate 74

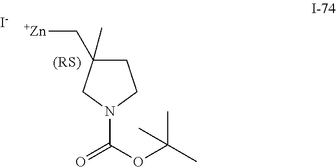

Intermediate 74 was prepared following an analogous procedure to the one described for the synthesis of intermediate 72 using intermediate 81 as starting material.

### Preparation of Intermediate 75

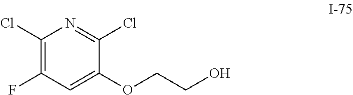 [SMILES: OCCOc1cc(F)c(Cl)nc1Cl]

(2-Bromoethoxy)-tert-butyldimethylsilane (CAS: 86864-60-0; 1.51 mL, 7.06 mmol) was added to a stirred suspension of 2,6-dichloro-5-fluoropyridin-3-ol (CAS: 2228660-663-5; 1.13 g, 6.21 mmol) and K₂CO₃ (1.22 g, 8.82 mmol) in DMF (5.95 mL). The reaction mixture was stirred at 90° C. for 16 h, diluted with water and extracted with EtOAc (twice). The combined organic layers were dried (Na₂SO₄), filtered and the solvents were evaporated in vacuo. The crude product was purified by flash column chromatography (SiO₂, EtOAc in heptane, gradient from 0/100 to 50/50). The desired fractions were collected and the solvents were evaporated in vacuo to afford intermediate 75 (1.095 g, 78%) as white solid.

### Preparation of Intermediate 76

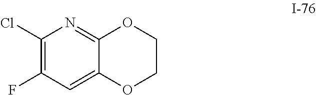 [SMILES: Fc1cc2c(nc1Cl)OCCO2]

To a mixture of intermediate 75 (1.30 g, 5.77 mmol) in t-BuOH (32.6 mL) was added t-BuOK (777 mg, 6.92 mmol). The reaction mixture was heated at 90° C. for 1 h, cooled down and the solvent was removed in vacuo. The residue was diluted with water and EtOAc. The mixture was filtered through a pad of Celite® and washed with EtOAc. The organic layer was washed with brine, dried (Na₂SO₄), filtered and concentrated in vacuo. The crude product was purified by flash column chromatography (SiO₂, EtOAc in heptane, gradient from 0/100 to 50/50). The desired fractions were collected and concentrated in vacuo to yield intermediate 76 (470 mg, 43%) as a white solid.

### Preparation of Intermediate 77

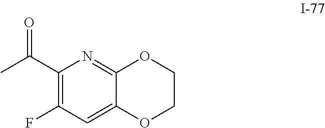

To a mixture of intermediate 76 (900 mg, 4.75 mmol) in toluene (16.7 mL) were added Pd(PPh₃)₂Cl₂ (366 mg, 0.52 mmol) and tributyl(1-ethoxyvinyl)tin (CAS: 97674-02-7; 2.25 mL, 6.65 mmol). The reaction mixture was stirred at 92° C. for 16 h. Then HCl (2N, 1 mL) was added and the mixture was stirred at room temperature for 3 h. The mixture was neutralized with NaHCO₃ (sat.) and extracted with EtOAc. The organic layer was dried (MgSO₄), filtered and concentrated in vacuo. The crude mixture was purified by flash column chromatography (SiO₂, EtOAc in heptane, gradient from 0/100 to 100/0). The desired fractions were collected and concentrated in vacuo to afford intermediate 77 (563 mg, 60%) as a brown solid.

### Preparation of Intermediate 78

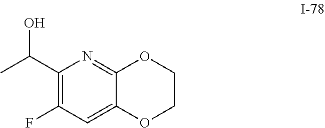

NaBH₄ (432 mg, 11.4 mmol) was added to a solution of intermediate 77 (563 mg, 2.86 mmol) in EtOH (13.4 mL) at 0° C. The reaction mixture was stirred at room temperature for 10 min. Water was added and the mixture was extracted with DCM. The combined organic layers were dried (Na₂SO₄), filtered and concentrated in vacuo to give intermediate 78 (465 mg, 81%) which was used as such in the next step.

### Preparation of Intermediate 79

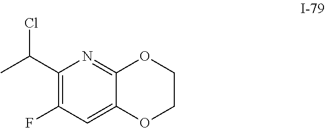

Thionyl chloride (0.64 mL, 8.74 mmol) was added to a solution of intermediate 78 (435 mg, 2.18 mmol) in DCM (14.6 mL) at 0° C. The reaction mixture was stirred at room temperature for 12 h. Water (20 mL) was added and the mixture was extracted with DCM (3×20 mL). The combined organic layers were dried (Na₂SO₄), filtered and evaporated in vacuo to afford intermediate 79 (453 mg, 87%, 91% purity) as a brown oil.

### Preparation of Intermediate 81

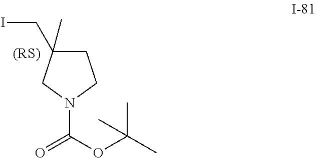

1-Boc-3-methylpyrrolidine-3-methanol (CAS: 1263506-20-2; 1 g, 4.64 mmol) was added portionwise to a solution of 12 (1.3 g, 5.1 mmol), PPh₃ (1.34 g, 5.1 mmol) and imidazole (474 mg, 6.96 mmol) in toluene (16.6 mL) at rt. The mixture was stirred for 16 h at 80° C. Then the mixture was cooled down to room temperature and EtOAc, water and Na₂S₂O₃ (aq. sat. soltn.) we added. The organic layer was separated and the volatiles were evaporated under vacuum. The residue thus obtained was purified by flash chromatography (silica gel, EtOAc in heptane, 0:100 to 10:90). The product containing fractions were evaporated in vacuo to yield intermediate 81 (1.1 g, 73%) as a colorless oil.

### Preparation of Intermediate 86

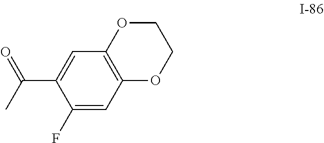

To a mixture of 1-(2,3-dihydro-1,4-benzodioxin-6-yl)ethenone (2.1 g, 11.78 mmol) in 1-butyl-3-methylimidazolium tetrafluoroborate (CAS 174501-65-6; 7 mL), N-fluoro-N′-(chloromethyl)triethylenediaminebis(tetrafluoroborate) (CAS140681-55-6; 10.43 g, 29.5 mmol) was added. The reaction mixture was heated at 70° C. for 16 h. Then it was cooled to rt, treated with water and extracted with EtOAc (2×15 mL). The combined organic layer was evaporated in vacuo to afford an oil which was purified by flash column chromatography (SiO₂, EtOAc in Heptane, 0/100 to 10/90). The desired fractions were concentrated to yield intermediate 86 (1.1 g, 48%) as white solid.

### Preparation of Intermediate 93

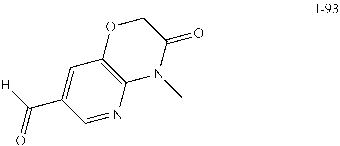

Sodium periodate (2.91 g, 13.6 mmol) followed by osmium tetroxide (0.472 mL, 0.035 mmol, 2.5% in t-BuOH) and 2,6-dimethylpyridine (0.71 mL, 6.11 mmol) were added to a stirred solution of intermediate 94 (508 mg, 2.67 mmol) in 1,4-dioxane (25 mL) and water (7.5 mL) in a sealed tube and under N₂ atmosphere. The mixture was stirred at rt for 16 h. The mixture was treated with water, filtered and washed with EtOAc. The filtrate was extracted with additional EtOAc. The organic layer was separated, dried (MgSO₄), filtered and the solvents evaporated in vacuo. The crude product was purified by flash column chromatography (SiO₂, EtOAc in DCM 0/100 to 100/0). The desired fractions were collected and concentrated in vacuo to yield intermediate 93 (230 mg, 45%) as a white solid.

### Preparation of Intermediate 94

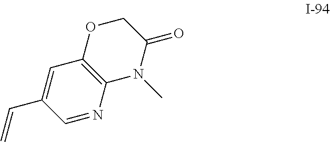

Potassium carbonate (7.5 mL, 10% aq soltn) followed by 4,4,5,5-tetramethyl-2-vinyl-1,3,2-dioxaborolane (CAS: 75927-49-0; 0.65 mL, 3.83 mmol) and Pd(PPh₃)₄ (365 mg, 0.31 mmol) were added to a stirred solution of 7-bromo-4-methyl-2,3-dihydro-4H-pyrido[3,2-b][1,4]oxazin-3-one (CAS: 122450-97-9) in 1,4-dioxane (7.5 mL) in a sealed tube and under N₂ atmosphere. The mixture was stirred at 150° C. for 15 min under microwave irradiation. The mixture was treated with water and extracted with DCM. The organic layer was separated, dried (MgSO₄), filtered and the solvents evaporated in vacuo. The crude product was purified by flash column chromatography (SiO₂, EtOAc in heptane 0/100 to 100/0). The desired fractions were collected and concentrated in vacuo to yield intermediate 94 (516 mg, 87%) as a white solid.

### Preparation of Intermediate 96

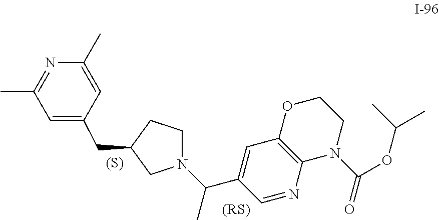

Titanium(IV) isopropoxide (578.5 μL, 1.98 mmol) was added dropwise to a stirred solution of intermediate 25 (125 mg, 0.66 mmol) and tert-butyl 7-formyl-2H-pyrido[3,2-b][1,4]oxazine-4(3H)-carboxylate (CAS: 1287312-62-2, 216.36 mg, 0.82 mmol) in DCM (3.98 mL) in a sealed tube and under N₂. The mixture was stirred at rt for 16 h. The mixture was cooled at 0° C. and methyl magnesium bromide (1.4 M in THF, 2.31 mL, 3.24 mmol) was added dropwise over 10 min. The mixture was stirred at rt for 19 h. The mixture was treated with sat NH₄Cl and DCM and filtered through a pad of diatomaceous earth and washed with more DCM. The filtrate was extracted with additional DCM. The organic layer was separated, dried (MgSO₄), filtered and the solvents evaporated in vacuo. The crude product was purified by flash column chromatography (silica; MeOH in DCM 0/100 to 10/90). The desired fractions were collected and evaporated to yield intermediate 96 (80.2 mg, 28%) as a yellow sticky solid.

### Preparation of Intermediate 97

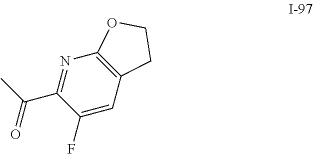

To a mixture of intermediate 98 (340 mg, 2.071 mmol) in dry THF (20 mL), methyl magnesium bromide (2.071 mL, 2.9 mmol, 1.4 M in THF) was added at 0° C. After completion of the addition, the reaction was stirred for 16 h at rt. The mixture was quenched with 1M aq HCl and stirred for 30 min, then the crude was basified with NH₄OH until pH 8. The solution was extracted with EtOAc (2×5 mL) The combined organic extracts were dried (Na₂SO₄), filtered and evaporated to dryness to give a residue that was purified by flash column chromatography (SiO₂, EtOAc in heptane 0/100 to 20/80). The desired fractions were collected and concentrated to yield intermediate 97 (150 mg, 40%) as a colorless oil.

### Preparation of Intermediate 98

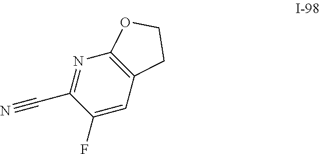 [SMILES: N#Cc1nc2c(cc1F)CCO2]

To a mixture of intermediate 99 (400 mg, 2.57 mmol) in acetonitrile (7 mL), trimethylsilyl cyanide (CAS:7677-24-9; 1.29 mL, 10.3 mmol) and triethylamine (0.9 mL, 6.47 mmol) were added. The mixture was stirred at 90° C. for 24 h. The mixture was cooled and treated with water and extracted with EtOAc (2×10 mL). The combined organic extracts were dried over MgSO₄ and the solvent was evaporated in vacuo to give a residue that was purified by flash column chromatography (SiO₂, EtOAc in heptane 0/100 to 30/60). The desired fractions were collected and concentrated in vacuo to intermediate 98 (320 mg, 76%) as an oil.

### Preparation of Intermediate 99

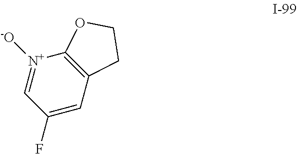

To a mixture of 5-fluoro-2,3-dihydrofuro[2,3-b]pyridine (CAS: 1356542-41-0; 500 mg, 3.6 mmol) in DCM (15 mL), meta-chloroperbenzoic acid (806 mg, 4.7 mmol) was added at rt. The mixture was stirred at 25° C. for 36 h. The solvent was removed in vacuo, and the residue thus obtained was purified by silica gel column chromatography (silica; EtOAc in heptane 0/100 to 30/70 then DCM in MeOH 0/100 to 6/94). The desired fractions were collected and concentrated in vacuo to afford intermediate 99 (400 mg, 72%) as white solid.

### Preparation of Intermediate 100

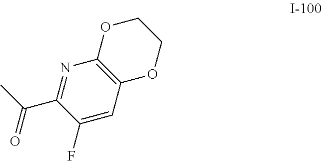

To a mixture of intermediate 101 (1.6 g, 5.7 mmol) in toluene (15 mL), bis(triphenylphosphine)palladium(II) dichloride (400 mg, 0.57 mmol) and tributyl(1-ethoxyvinyl)tin (CAS: 97674-02-7; 2.5 mL, 7.4 mmol) were added. The mixture was heated at 92° C. for 16 h, then the crude was cooled and treated with aqueous 2N HCl (5 mL) and the mixture was stirred for 2 h. The crude was neutralised with an aqueous saturated solution of NaHCO₃ and extracted with EtOAc and the combined organic layers were evaporated in vacuo. The crude was purified by flash column chromatography (SiO₂, MeOH in DCM 0/100 to 5/95). The desired fractions were collected and concentrated in vacuo to yield intermediate 100 (850 mg, 76%) as orange solid.

### Preparation of Intermediate 101

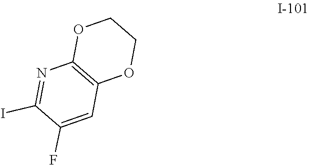 [SMILES: Fc1cc2c(nc1I)OCCO2]

To a mixture of intermediate 102 (5 g, 12.2 mmol) in t-BuOH (6.91 mL), potassium tert-butoxide (206 mg, 1.83 mmol) was added at rt. The mixture was heated at 90° C. for 3 h. After cooling, the solvent was removed in vacuo and the residue was diluted with water. The aqueous solution was extracted with EtOAc (3×12 mL). The combined organic layers were washed with brine (2×10 mL), separated and dried over anhydrous Na2SO4 and concentrated. The crude was purified by flash column chromatography (SiO2, MeOH in DCM 0/100 to 5/95). The desired fractions were collected and concentrated in vacuo to yield intermediate 101 (1.6 g, 47%) as white solid.

### Preparation of Intermediate 102

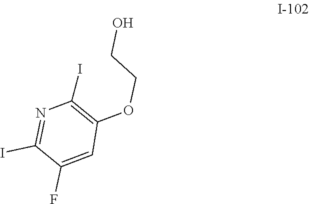 [SMILES: OCCOc1cc(F)c(I)nc1I]

To a mixture of intermediate 103 (8 g, 15.3 mmol) in THF (120 mL), tetrabutylammonium fluoride (15.3 mL, 15. mmol, 1M solution in THF) was added the mixture was stirred for 3 h at rt. Water was added and the crude was extracted with EtOAc. The organic phase was dried (Na2SO4) and evaporated in vacuo to afford an oil which was purified by column chromatography (SiO2, MeOH in DCM, 0/100 to 5/95). The desired fractions were concentrated to yield intermediate 102 (5.8 g, 92%) as oil.

### Preparation of Intermediate 103

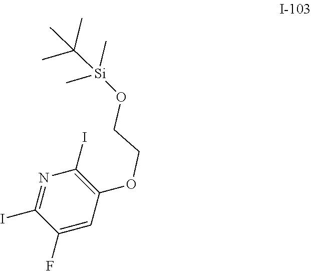 [SMILES: CC(C)(C)[Si](C)(C)OCCOc1cc(F)c(I)nc1I]

A mixture of intermediate 104 (6.1 g, 16.7 mmol), (2-bromoethoxy)dimethyl-tert-butylsilane (4.4 gm 18.4 mmol), and potassium tert-butoxide (5.08 g, 36.78 mmol) in DMF (15 mL) was heated at 90° C. for 5 h. The crude was cooled and treated with water and extracted with EtOAc (2×20 mL). The combined organic extracts were evaporated in vacuo to afford a residue that was purified by column chromatography (SiO2, EtOAc in heptane, 0/100 to 20/80). The desired fractions were concentrated in vacuo to yield intermediate 103 (8.1 g, 93%) as an oil.

### Preparation of Intermediate 104

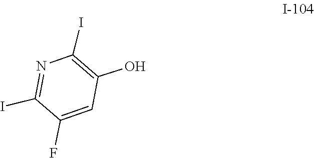

To a solution of 3-fluoro-5-hydroxypyridine (2 g, 17.7 mmol) in Na₂CO₃ (30 mL, aq. sat. soltn.) and water (10 mL), I₂ (9.2 g, 36.25 mmol) was added and the mixture was stirred for 16 h at rt. The reaction mixture was quenched with an aqueous saturated solution of Na₂S₂O₃ and the solution pH was adjusted to pH=5 by addition of aqueous HCl. The reaction mixture was extracted with EtOAc (3×70 mL) and the combined organic layer was dried over MgSO₄, filtered and evaporated in vacuo to yield intermediate 104 (6.02 g, 93%) as a yellow solid.

### Preparation of Intermediate 107

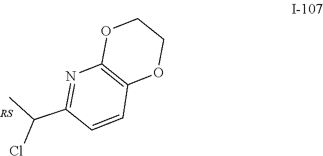

Thionyl chloride (6.51 mL, 89 mmol) was added to a solution of intermediate 108 (4.04 g, 22.3 mmol) in DCM (150 mL) at 0° C. The mixture was stirred at rt for 12 h. Water (80 mL) was added and the mixture was extracted with DCM (80 mL×3). The combined organic layers were dried (Na₂SO₄), filtered and evaporated in vacuo to yield crude intermediate 107 (3.53 g, 79%) as a brown oil that solidified upon standing.

### Preparation of Intermediate 108

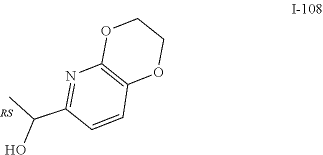

Sodium borohydride (3.54 g, 94 mmol) was added to a solution of 1-(2,3-dihydro-[1,4]dioxino[2,3-b]pyridin-6-yl)ethenone (CAS: 1254044-25-1; 4.5 g, 23.4 mmol) in EtOH (109 mL) at 0° C. The mixture was stirred at rt for 10 min. Water was added and the mixture was extracted with DCM (80 mL×3). The organic layers were combined, dried (Na2SO4), filtered and concentrated in vacuo to yield intermediate 108 (4.04 g, 95%) as a pale yellow oil.

### Preparation of Intermediate 118

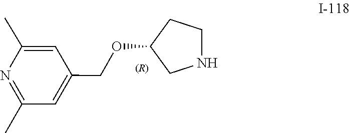

Intermediate 118 was prepared following an analogous procedure to the one described for the synthesis of intermediate 20 using intermediate 119 as starting material.

### Preparation of Intermediate 119

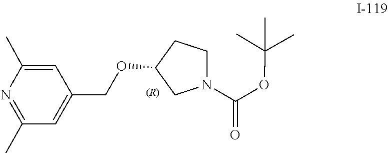

Intermediate 119 was prepared following an analogous procedure to the one described for the synthesis of intermediate 44 using (R)-1-Boc-3-hydroxypyrrolidine (CAS: 109431-87-0) and 4-chloromethyl-2,6-dimethylpyridine (CAS: 120739-87-9) as starting materials.

### Preparation of Intermediate 120

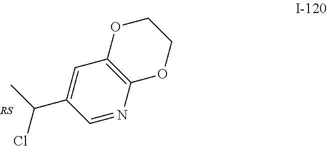

Intermediate 120 was prepared following an analogous procedure to the one described for the synthesis of intermediate 107 using intermediate 121 as starting material.

### Preparation of Intermediate 121

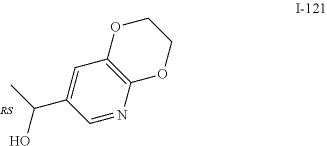

Intermediate 121 was prepared following an analogous procedure to the one described for the synthesis of intermediate 108 using 1-(2,3-dihydro-[1,4]dioxino[2,3-b]pyridin-7-yl)-ethanone (CAS: 1254044-15-9) as starting material.

### Preparation of Intermediate 122

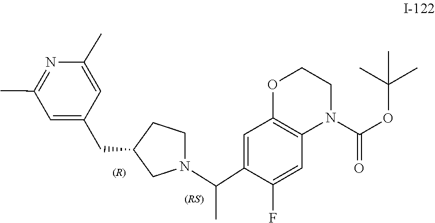

Intermediate 122 was prepared following an analogous procedure to the one described for the synthesis of intermediate 96 using intermediate 27 and intermediate 123 as starting materials.

### Preparation of Intermediate 123

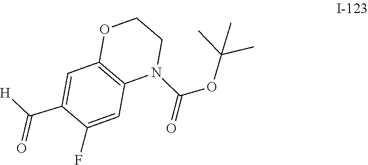 [SMILES: CC(C)(C)OC(=O)N1CCOc2cc(C=O)c(F)cc21]

Intermediate 123 was prepared following an analogous procedure to the one described for the synthesis of intermediate 93 using intermediate 124 as starting material.

### Preparation of Intermediate 124

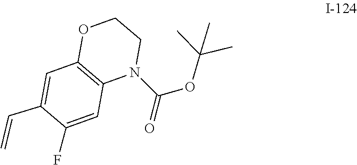 [SMILES: C=Cc1cc2c(cc1F)N(C(=O)OC(C)(C)C)CCO2]

Intermediate 124 was prepared following an analogous procedure to the one described for the synthesis of intermediate 94 using intermediate 125 as starting material.

### Preparation of Intermediate 125

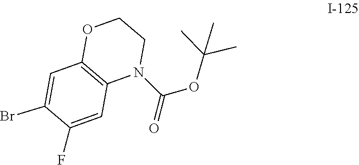 [SMILES: CC(C)(C)OC(=O)N1CCOc2cc(Br)c(F)cc21]

Borane dimethyl sulphide complex (1.65 mL, 17.4 mmol) was added dropwise to a stirred suspension of 7-bromo-6-fluoro-2H-benzo[b][1,4]oxazin-3(4H)-one (CAS: 1260829-35-3; 2.1 g, 8.53 mmol) in THF (44 mL) in a round-bottom flask under a condenser and under N2 atmosphere. The mixture was stirred at reflux temperature for 2 h. The mixture was cooled at 0° C. and MeOH (12 mL) was added dropwise. The mixture was stirred at rt for 1 h. The solvent was evaporated in vacuo. The crude taken up in THF (44 mL) and cooled at 0° C. Boc-anhydride (CAS: 24424-99-5; 2.65 mL, 12.4 mmol) was added in one portion followed by lithium bis(trimethylsilyl)amide (12.1 mL, 12.1 mmol, 1M solution in THF) dropwise and the mixture was stirred at 0° C. for 1 h and at rt for 60 h. The mixture was treated with aq sat NH₄Cl and extracted with EtOAc. The organic layer was separated, dried (MgSO4), filtered and the solvents evaporated in vacuo. The crude product was purified by flash column chromatography (SiO2, EtOAc in heptane 0/100 to 70/30). The desired fractions were collected and concentrated in vacuo to yield intermediate 125 (2.8 g, 99%) as a yellow oil

### Preparation of Intermediate 126

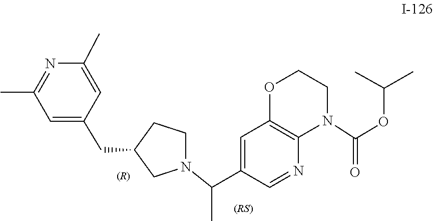

Intermediate 126 was prepared following an analogous procedure to the one described for the synthesis of intermediate 96 using intermediate 24 and tert-butyl 7-formyl-2H-pyrido[3,2-b][1,4]oxazine-4(3H)-carboxylate (CAS: 1287312-62-2) as starting materials.

### Preparation of Intermediate 127

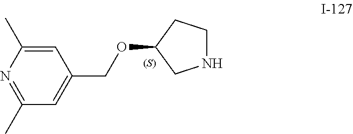

Intermediate 127 was prepared following an analogous procedure to the one described for the synthesis of intermediate 20 using intermediate 128 as starting material.

### Preparation of Intermediate 128

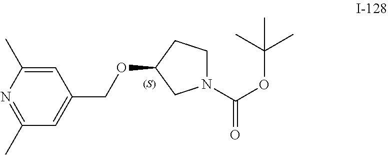

Intermediate 128 was prepared following an analogous procedure to the one described for the synthesis of intermediate 44 using (S)-1-Boc-3-(hydroxymethyl)pyrrolidine (CAS: 109431-87-0) and 4-chloromethyl-2,6-dimethylpyridine (CAS: 120739-87-9) as starting materials.

### Preparation of Intermediate 129

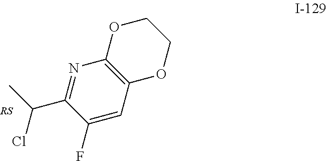

Intermediate 129 was prepared following an analogous procedure to the one described for the synthesis of intermediate 107 using intermediate 130 as starting material.

### Preparation of Intermediate 130

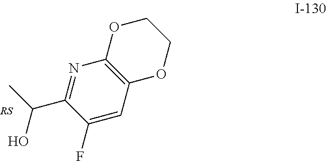

Intermediate 130 was prepared following an analogous procedure to the one described for the synthesis of intermediate 108 using intermediate 100 as starting material.

### Preparation of Intermediate 131

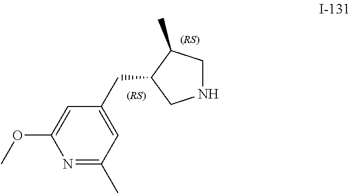

Intermediate 131 was prepared following an analogous procedure to the one described for the synthesis of intermediate 23 using intermediate 132 as starting material.

### Preparation of Intermediate 132

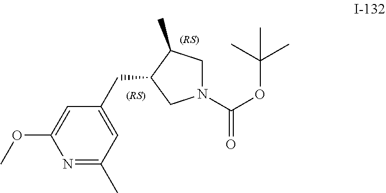

Intermediate 132 was prepared following an analogous procedure to the one described for the synthesis of intermediate 48 using intermediate 133 and 4-bromo-2-methoxy-6-methylpyridine (CAS: 1083169-00-9) as starting materials.

### Preparation of Intermediate 133

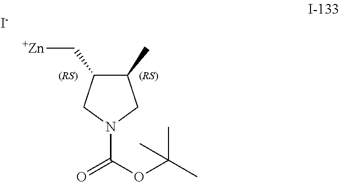

Intermediate 133 was prepared following an analogous procedure to the one described for the synthesis of intermediate 72 using intermediate 134 as starting material.

### Preparation of Intermediate 134

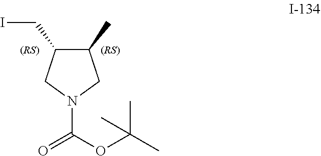

Intermediate 134 was prepared following an analogous procedure to the one described for the synthesis of intermediate 81 using intermediate trans-tert-butyl-3-(hydroxymethyl)-4-pyrrolidine-1-carboxylate as starting material.

### Preparation of Intermediate 135

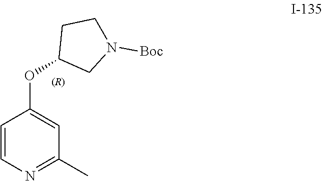

To a solution of (R)-(−)-N-Boc-3-pyrrolidinol (CAS: 103057-44-9; 150 mg, 0.80 mmol) in anhydrous DMF (2.02 mL) were added NaH (60% dispersion in mineral oil, 38.5 mg, 0.96 mmol) and 15-crown-5 (0.2 mL, 0.96 mmol) at 0° C. under N₂ atmosphere. 4-Chloro-2-methylpyridine (CAS: 3678-62-4; 97.84, 0.88 mmol) was added and the reaction mixture was stirred at 60° C. for 16 h. Additional amount of NaH (60% dispersion in mineral oil, 1 eq) was added and the reaction mixture was stirred overnight at 60° C. Again NaH (60% dispersion in mineral oil, 1 eq) was added and the reaction mixture was stirred for another 4 h at 60° C. Water was added at 0° C. and the mixture was extracted with EtOAc. The organic layer was dried (Na₂SO₄), filtered and the solvents were evaporated in vacuo. The crude mixture was purified by flash column chromatography (silica, EtOAC in heptane, gradient from 0/100 to 80/20). The desired fractions were collected and the solvents were evaporated in vacuo to give intermediate 135 (178.9 mg, 80%) as a yellow oil.

### Preparation of Intermediate 136

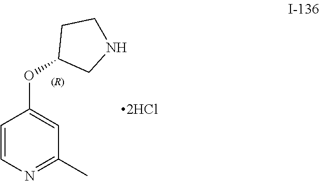

HCl (4M in 1,4-dioxane, 1.93 mL, 7.71 mmol) was added to a stirred solution of intermediate 135 (179 mg, 0.64 mmol) in 1,4-dioxane (5.5 mL). The reaction mixture was stirred at room temperature for 3 h and the solvent was evaporated in vacuo to give intermediate 136 (2HCl salt, 191 mg) which was used in the next step without any purification.

### Preparation of Intermediate 137

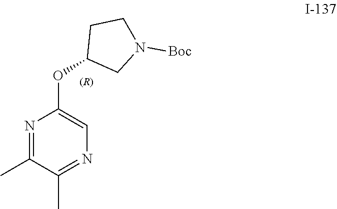

Intermediate 137 was prepared following an analogous procedure to the one described for the synthesis of intermediate 135 using (R)-(−)-N-boc-3-pyrrolidinol (CAS: 103057-44-9) and 5-chloro-2,3-dimethylpyrazine (CAS: 182500-28-3) as starting materials. The crude was purified by flash column chromatography (silica, EtOAc in heptane, gradient from 0/100 to 80/20). The desired fractions were collected and the solvents were evaporated in vacuo to afford intermediate 137 (198 mg, 84%) as a light yellow oil.

### Preparation of Intermediate 138

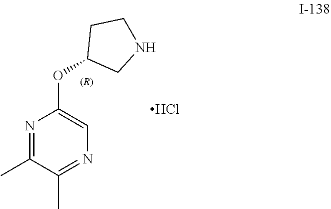

Intermediate 138 was prepared following an analogous procedure to the one described for the synthesis of intermediate 136 using intermediate 137 as starting material. The crude product (HCl salt, 190 mg) was used in the next step without any purification.

### Preparation of Intermediate 139

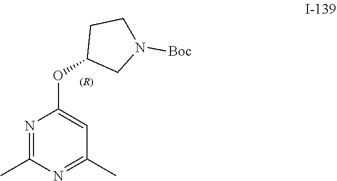

Intermediate 139 was prepared following an analogous procedure to the one described for the synthesis of intermediate 135 using (R)-(−)-N-boc-3-pyrrolidinol (CAS: 103057-44-9) and 4-chloro-2,6-dimethylpyrimidine (CAS: 182500-28-3) as starting materials. The crude was purified by flash column chromatography (silica, EtOAc in heptane, gradient from 0/100 to 80/20). The desired fractions were collected and the solvents were evaporated in vacuo to afford intermediate 139 (167 mg, 71%) as a light yellow oil.

### Preparation of Intermediate 140

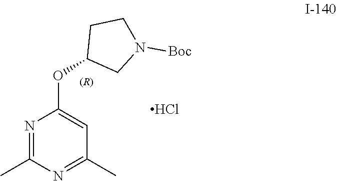

Intermediate 140 was prepared following an analogous procedure to the one described for the synthesis of intermediate 136 using intermediate 139 as starting material. The crude product (HCl salt, 160 mg) was used in the next step without any purification.

### Preparation of Intermediate 141

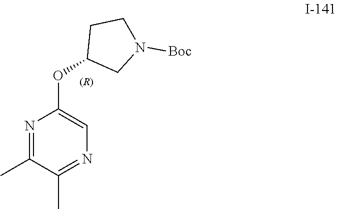

To a solution of 5,6-dimethylpyridin-3-ol (CAS: 61893-00-3; 100 mg, 0.81 mmol) in DMSO (2.56 mL) under N₂ atmosphere were added Cs₂CO₃ (337 mg, 1.03 mmol) and (R)-tert-butyl 3-(tosyloxy)pyrrolidine-1-carboxylate (CAS: 139986-03-1; 252 mg, 0.74 mmol). The reaction mixture was stirred at 60° C. for 4 h. Water was added and the mixture was extracted with EtOAc. The organic layer was dried (Na₂SO₄), filtered and concentrated in vacuo. The crude mixture was purified by flash column chromatography (silica, EtOAc in heptane, gradient from 0/100 to 80/20). The desired fractions were collected and the solvents were evaporated in vacuo to afford intermediate 141 (146 mg, 68%) as a light yellow solid.

### Preparation of Intermediate 142

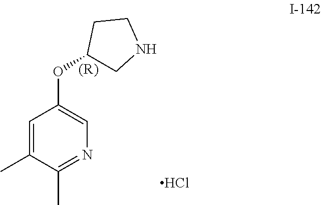

Intermediate 142 was prepared following an analogous procedure to the one described for the synthesis of intermediate 136 using intermediate 141 as starting material. The crude product (HCl salt, 151 mg) was used in the next step without any purification.

### Preparation of Intermediate 143

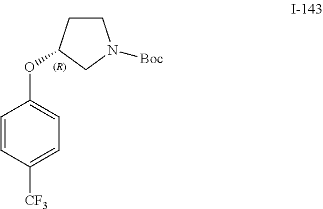

Intermediate 143 was prepared following an analogous procedure to the one described for the synthesis of intermediate 141 using R)-tert-butyl 3-(tosyloxy)pyrrolidine-1-carboxylate (CAS: 139986-03-1) and 5-hydroxy-2-(trifluoromethyl)pyridine (CAS: 216766-12-0) as starting materials.

The crude mixture was purified by flash column chromatography (silica, EtOAc in heptane, gradient from 0/100 to 80/20). The desired fractions were collected and the solvents were evaporated in vacuo to afford intermediate 143 (172 mg, 78%) as a light yellow oil.

### Preparation of Intermediate 144

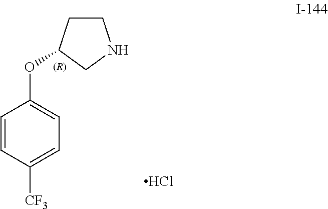

Intermediate 144 was prepared following an analogous procedure to the one described for the synthesis of intermediate 136 using intermediate 143 as starting material. The crude product (HCl salt, 144 mg) was used in the next step without any purification.

### Preparation of Intermediate 145

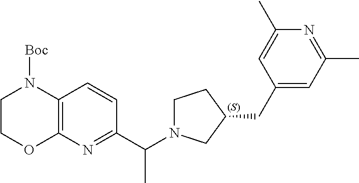

Ti(Oi-Pr)₄ (CAS: 546-68-9; 0.58 mL, 1.98 mmol) was added dropwise to a stirred mixture of intermediate 25 (125 mg, 0.66 mmol) and intermediate 58 (216 mg, 0.82 mmol) in DCM (3.98 mL) in a sealed tube and under N₂ atmosphere. The reaction mixture was stirred at room temperature for 16 h, cooled to 0° C. and methylmagnesium bromide (1.4M in THF, 2.31 mL, 3.24 mmol) was added dropwise over 10 min. The reaction mixture was stirred at room temperature for 19 h. The mixture was treated with NH₄Cl (sat. solution) and DCM, filtered through a pad of Celite® and washed with DCM. The filtrate was extracted with DCM. The organic layer was dried (MgSO₄), filtered and the solvents were evaporated in vacuo. The crude product was purified by flash column chromatography (silica, MeOH in DCM, gradient from 0/100 to 10/90). The desired fractions were collected and evaporated in vacuo to give intermediate 145 (118 mg, 40%) as a brown oil.

### Preparation of Intermediate 146

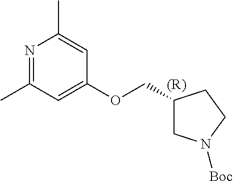

NaH (60% dispersion in mineral oil, 2338 mg, 5.96 mmol) was added to a solution of (R)-3-hydroxymethyl-pyrrolidine-1-carboxylic acid tert-butyl (CAS: 138108-72-2; 1.00 g, 4.97 mmol) in DMF (10 mL) at 0° C. under nitrogen atmosphere. The reaction mixture was stirred at 0° C. for 30 min and 4-chloro-2,6-dimethylpyridine (CAS: 3512-75-2; 697 mg, 5.47 mmol) was added dropwise. The reaction mixture was stirred at 0° C. for 1 h, then at 80° C. for 20 h. The reaction was quenched with NH₄Cl (sat.) and extracted with EtOAc. The organic layer was separated, dried (MgSO₄), filtered and evaporated in vacuo. The crude product was purified by flash column chromatography (silica, EtOAc in heptane, gradient from 50/50 to 100/0). The desired fractions were collected and concentrated in vacuo to yield intermediate 146 (1.40 g, 92%) as an oil.

### Preparation of Intermediate 147

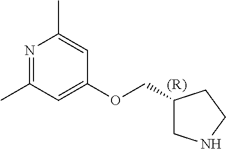

A solution of intermediate 146 (1.40 g, 4.57 mmol in MeOH (30 mL) was added to a reactor containing Amberlyst®15 hydrogen form (CAS: 39389-20-3; 4.86 g). The mixture was shaken in a solid phase reactor at room temperature for 16 h. The resin was washed with MeOH (the fraction was discarded) and then with NH₃ (7N in MeOH). The filtrates were concentrated in vacuo to give intermediate 147 (500 mg, 53%) as a pale brown oil.

### Preparation of Intermediate 148

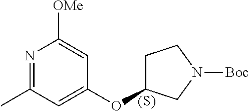

Intermediate 148 was prepared following an analogous procedure to the one described for the synthesis of intermediate 146 using (S)-(+)-N-boc-3-pyrrolidinol (CAS: 101469-92-5) and 4-bromo-2-methoxy-6-methylpyridine (CAS: 1083169-00-9) as starting materials.

### Preparation of Intermediate 149

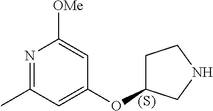

Intermediate 149 was prepared following an analogous procedure to the one described for the synthesis of intermediate 147 using intermediate 148 as starting material.

### Preparation of Final Compounds

### E1. Preparation of Product 1

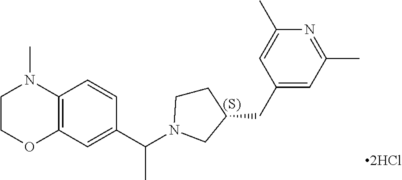

Sodium cyanoborohydride (CAS: 25895-60-7; 71.6 mg, 1.14 mmol) was added to a mixture of intermediate 54.2HCl (250 mg, 0.95 mmol), intermediate 25 (182 mg, 0.95 mmol), Ti(Oi-Pr)₄ (CAS: 546-68-9; 0.28 mL, 0.95 mmol) and Et₃N (0.39 mL, 2.85 mmol) in DCM (3.12 mL) at room temperature. The reaction mixture was stirred at 80° C. for 16 h. Water was added and the product was extracted with DCM. The organic layer was separated, dried (Na₂SO₄), filtered and concentrated in vacuo. The crude mixture was purified by flash column chromatography (silica, NH₃ (7M in MeOH) in DCM, gradient from 0/100 to 5/95). The desired fractions were collected and concentrated in vacuo. A second purification was performed by RP HPLC (stationary phase: C18 XBridge 30×100 mm 5 μm), mobile phase: NH₄HCO₃ (0.25% solution in water)/CH₃CN, gradient from 67/33 to 50/50) to afford a colorless oil.

The product (220 mg) was dissolved in MeOH and treated with HCl (6M in i-PrOH, 0.48 mL, 2.85 mmol). The mixture was stirred at room temperature for 2 h and concentrated in vacuo to give product 1 (221 mg, 53%) as a white solid.

### E2. Preparation of Product 2

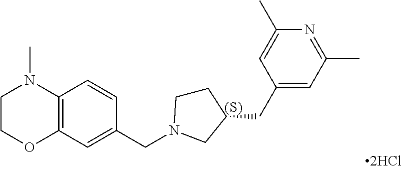

Sodium triacetoxyborohydride (CAS: 56553-60-7; 302 mg, 1.43 mmol) was added to a mixture of intermediate 25.2HCl ((250 mg, 0.95 mmol), 4-methyl-3,4-dihydro-2H-1,4-benzoxazine-7-carbaldehyde (CAS: 141103-93-7; 168 mg, 0.95 mmol) and Et₃N (0.40 mL, 2.85 mmol) in MeOH (3.08 mL). The reaction mixture was stirred at room temperature for 16 h. Water was added and the product was extracted with EtOAc. The organic layer was dried (Na₂SO₄), filtered and concentrated in vacuo. The crude mixture was purified by flash column chromatography (silica, NH₃ (7M in MeOH) in DCM, gradient from 0/100 to 5/95). The desired fractions were collected and concentrated in vacuo. A second purification was performed by RP HPLC (stationary phase: C18 XBridge 30×100 mm 5 μm), mobile phase: NH₄HCO₃ (0.25% solution in water)/CH₃CN, gradient from 80/20 to 60/40). The residue (180 mg) was dissolved in MeOH and treated with HCl (6M in i-PrOH, 0.16 mL, 0.95 mmol). The mixture was stirred at room temperature for 2 h and concentrated in vacuo to afford product 2 (198 mg, 49%) as a grey solid.

### E3. Preparation of Product 3

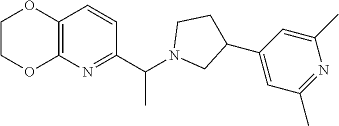 [SMILES: Cc1cc(C2CCN(C(C)c3ccc4c(n3)OCCO4)C2)cc(C)n1]

2,3-Dihydro-[1,4]dioxino[2,3-b]pyridine-6-carbaldehyde (CAS: 615568-24-6; 85.9 mg, 0.52 mmol) and Ti(Oi-Pr)₄ (CAS: 546-68-9; 0.37 mL, 1.23 mmol) were added to a stirred solution of intermediate 17.HCl (65.0 mg, 0.37 mmol) in DCM (1.28 mL) at room temperature and under N₂ atmosphere. The reaction mixture was stirred for 16 h, cooled to 0° C. and methylmagnesium bromide (1.4M in THF and toluene, 1.53 mL, 2.14 mmol) was added portionwise. The reaction mixture was stirred at this temperature for 15 min and at room temperature for 16 h. The mixture was treated with NH₄Cl (sat. solution), diluted with DCM and filtered through a pad of diatomaceous earth. The organic layer was separated, dried (MgSO₄), filtered and the solvents were evaporated in vacuo. The crude product was purified by flash column chromatography (silica, MeOH in EtOAc, gradient from 0/100 to 10/90). The desired fractions were collected and concentrated in vacuo. A second purification was performed by RP HPLC (stationary phase: C18 XBridge 30×100 mm 5 μm), mobile phase: NH₄HCO₃ (0.25% solution in water)/CH₃CN, gradient from 80/20 to 60/40) to give product 3 (14 mg, 11%) as a pale yellow oil.

### E4. Preparation of Product 4

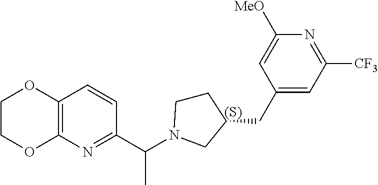

Product 4 was prepared following an analogous procedure to the one described for the synthesis of product 3 using 2,3-dihydro-[1,4]dioxino[2,3-B]pyridine-6-carbaldehyde (CAS: 615568-24-6) and intermediate 11.HCl. Intermediate 11.HCl was dissolved in MeOH and passed through an Isolute SCX-2 cartridge, eluting the product with NH₃ (7N in MeOH) prior to its use in the reaction.

The crude product was purified by flash column chromatography (silica, MeOH in EtOAc, gradient from 0/100 to 10/90). The desired fractions were collected and concentrated in vacuo. A second purification was performed by RP HPLC (stationary phase: C18)(Bridge 30×100 mm 5 μm), mobile phase: NH₄HCO₃ (0.25% solution in water)/CH₃CN, gradient from 54/46 to 36/64). The residue (36 mg) was treated with EtOAc and H₂O. The organic layer was separated, dried (Na₂SO₄), filtered and the solvent was evaporated in vacuo to give product 4 (30 mg, 21%) as a colorless film.

### E5. Preparation of Product 5

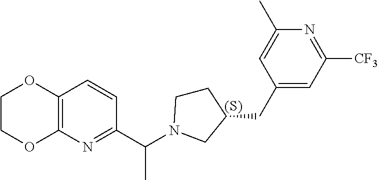

Product 5 was prepared following an analogous procedure to the one described for the synthesis of product 3 using 2,3-dihydro-[1,4]dioxino[2,3-B]pyridine-6-carbaldehyde (CAS: 615568-24-6) and intermediate 14.

The crude product was purified by flash column chromatography (silica, MeOH in EtOAc, gradient from 0/100 to 10/90). The desired fractions were collected and concentrated in vacuo. A second purification was performed by RP HPLC (stationary phase: C18)(Bridge 30×100 mm 5 μm), mobile phase: NH₄HCO₃ (0.25% solution in water)/CH₃CN, gradient from 54/46 to 36/64). The residue (65 mg) was treated with EtOAc and H₂O. The organic layer was separated, dreed (Na₂SO₄), filtered and the solvent was evaporated in vacuo to afford product 5 (50 mg, 30%) as a colorless film.

### E6. Preparation of Product 6

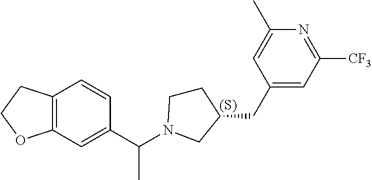

Product 6 was prepared following an analogous procedure to the one described for the synthesis of product 3 using intermediate 55 and intermediate 14.

The crude product was purified by flash column chromatography (silica, MeOH in EtOAc, gradient from 0/100 to 10/90). The desired fractions were collected and concentrated in vacuo to afford product 6 (115 mg, 72%) as a colorless oil.

### E7. Preparation of Product 7

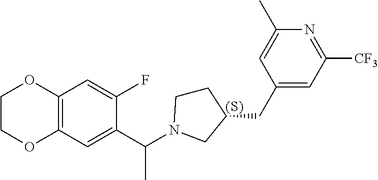

Ti(Oi-Pr)₄ (CAS: 546-68-9; 0.12 mL, 0.41 mmol) and sodium cyanoborohydride (CAS: 25895-60-7; 30.9 mg, 0.49 mmol) were added sequentially to a mixture of intermediate 14 (100 mg, 0.41 mmol) and intermediate 86 (80.3 mg, 0.41 mmol) in DCE (1.66 mL) at room temperature. The reaction mixture was stirred at 80° C. for 16 h in a sealed tube. The mixture was treated with NaHCO₃ (sat. solution), diluted with DCM and filtered through Celite®. The organic layer separated, dried (Na₂SO₄), filtered and the solvent was evaporated in vacuo. The crude product was purified by flash column chromatography (silica, MeOH in EtOAc, gradient from 0/100 to 10/90). The desired fractions were collected and evaporated in vacuo to afford product 7 (130 mg, 75%) as a pale yellow oil.

### E8. Preparation of Product 8

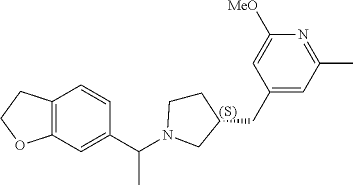

Intermediate 55 (80.4 mg, 0.54 mmol) and Ti(Oi-Pr)₄ (CAS: 546-68-9; 0.34 mL, 1.16 mmol) were added to a stirred solution of intermediate 26 (80.0 mg, 0.39 mmol) in DCM (1.58 mL) at room temperature and under N₂ atmosphere. The reaction mixture was stirred for 16 h, cooled to 0° C. and THF (0.45 mL) was added, followed by methylmagnesium bromide (1.4M in THF and toluene, 1.39 mL, 1.94 mmol) dropwise. The reaction mixture was stirred at this temperature for 25 min and at room temperature for 2 h. The mixture was treated with NH₄Cl (sat. solution) and extracted with DCM. The organic layer was dried (Na₂SO₄), filtered and the solvent was evaporated in vacuo. The crude product was purified by flash column chromatography (SiO₂ amino functionalized, MeOH in DCM, gradient from 0/100 to 4/96). The desired fractions were collected and concentrated in vacuo. A second purification was performed by RP HPLC (stationary phase: C18 XBridge 30×100 mm 5 μm), mobile phase: NH₄HCO₃ (0.25% solution in water)/CH₃CN, gradient from 54/46 to 36/64). The residue (80 mg) was washed with water and extracted with EtOAc. The organic layer was dried (Na₂SO₄), filtered and the solvent was evaporated in vacuo to give product 8 (78 mg, 57%) as an oil.

### E9. Preparation of Product 9

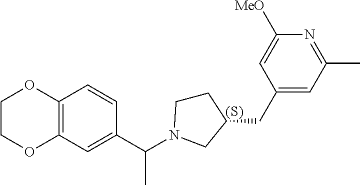

Product 9 was prepared following an analogous procedure to the one described for the synthesis of product 8 using intermediate 26 and 2,3-dihydro[1,4]dioxino[2,3-b]pyridine-6-carbaldehyde (CAS: 615568-24-6) as starting materials.

The crude product was purified by flash column chromatography (SiO₂ amino functionalized, MeOH in DCM, gradient from 0/100 to 4/96). The desired fractions were collected and concentrated in vacuo to afford product 9 (90 mg, 63%) as an oil.

### E10. Preparation of Products 10 and 11

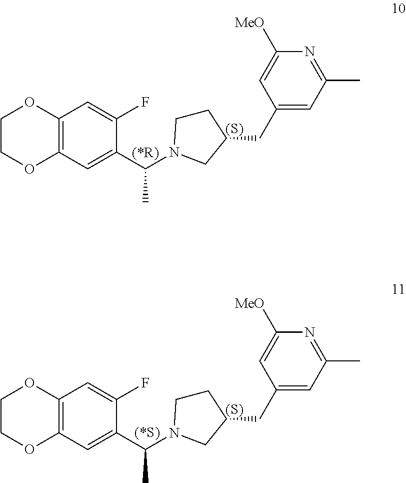

Ti(Oi-Pr)₄ (CAS: 546-68-9; 0.1 mL, 0.34 mmol) and sodium cyanoborohydride (CAS: 25895-60-7; 25.6 mg, 0.41 mmol) were added sequentially to a mixture of intermediate 26 (70.0 mg, 0.34 mmol) and intermediate 86 (66.6 mg, 0.34 mmol) in DCE (1.38 mL) at room temperature. The reaction mixture was stirred at 75° C. for 5 h in a sealed tube, and at 55° C. for 18 h. The mixture was treated with water, diluted with DCM and filtered through Celite®. The organic layer was separated, dried (Na₂SO₄), filtered and the solvent was evaporated in vacuo. The crude product was purified by flash column chromatography (silica, MeOH in DCM, gradient from 0/100 to 4/96). A second purification was performed via RP HPLC (stationary phase: C18 XBridge 30×100 mm 5 μm), mobile phase: NH₄HCO₃ (0.25% solution in water)/CH₃CN, gradient from 47/53 to 30/70) to give product 10 (25 mg 19%) and product 11 (30 mg, 23%) as oils.

### E11. Preparation of Product 12

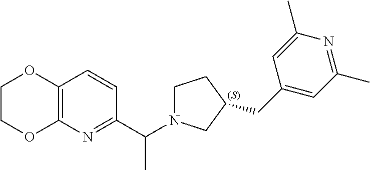

Ti(Oi-Pr)₄ (CAS: 546-68-9; 0.46 mL, 1.58 mmol) was added dropwise to a stirred mixture of intermediate 25 (100 mg, 0.53 mmol) and 2,3-dihydro-[1,4]dioxine[2,3-b]pyridine-6-carbaldehyde (CAS: 615568-24-6; 95.5 mg, 0.58 mmol) in THF (2.5 mL) in a sealed tube and under N₂ atmosphere. The reaction mixture was stirred for 16 h, cooled to 0° C. and methylmagnesium bromide (2.66 mmol) was added dropwise. The reaction mixture was stirred at 0° C. for 5 min and at room temperature for 18 h. The mixture was treated with NH₄Cl (sat. solution) and DCM, and filtered through a pad of Celite®. The filtrate was extracted with DCM. The organic layer was dried (MgSO₄), filtered and the solvents were evaporated in vacuo. The crude product was purified by flash column chromatography (SiO₂, NH₃ (7N in MeOH) in DCM, gradient from 0/100 to 3/97). A second purification was performed by RP HPLC (stationary phase: C18 XBridge 30×100 mm 5 μm), mobile phase: NH₄HCO₃ (0.25% solution in water)/CH₃CN, gradient from 60/40 to 37/63). The desired fractions were collected and evaporated in vacuo to afford product 12 (23.4 mg, 13%) as a yellowish oil.

### E12. Preparation of Products 13 and 14

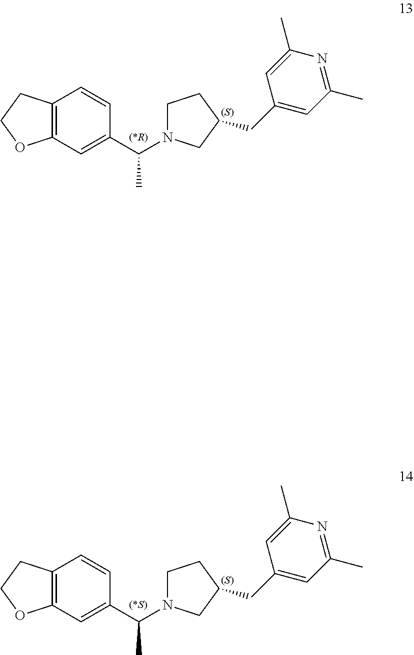

Products 13 and 14 were prepared following an analogous procedure to the one described for the synthesis of product 12 using intermediate 25 and intermediate 55 as starting materials.

The crude product was purified by flash column chromatography (SiO₂, NH₃ (7N in MeOH) in DCM, gradient from 0/100 to 3/97). A second purification was performed by RP HPLC (stationary phase: C18 XBridge 30×100 mm 5 μm), mobile phase: NH₄HCO₃ (0.25% solution in water)/CH₃CN, gradient from 60/40 to 37/63). The desired fractions were collected and evaporated in vacuo to give product 13 (15 mg, 8%) and product 14 (22.1 mg, 22%) as colorless oils.

### E13. Preparation of Product 15

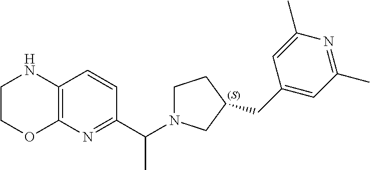

HCl (4M in 1,40-dioxane, 1.89 mL, 7.57 mmol) was added to intermediate 145 (118 mg, 0.26 mmol) and the reaction mixture was stirred at room temperature for 2 h. The solvent was evaporated in vacuo. The residue was purified using an Isolute® SCX-2 cartridge. The residue was washed with MeOH and the product was eluted with NH₃ (7N in MeOH). The desired fractions were collected and evaporated in vacuo. The residue was purified by flash column chromatography (silica, NH₃ (7N in MeOH) in DCM, gradient from 0/100 to 10/90). The desired fractions were collected and the solvents were evaporated in vacuo to give product 15 (53.5 mg, 58%).

### E14. Preparation of Product 16

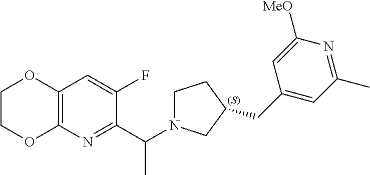

Sodium cyanoborohydride (CAS: 25895-60-7; 19.2 mg, 0.31 mmol) was added to a stirred mixture of intermediate 26 (30.0 mg, 0.15 mmol), intermediate 77 (34.4 mg, 0.18 mmol) and Ti(Oi-Pr)₄ (CAS: 546-68-9; 86.1 μL, 0.29 mmol) in anhydrous THF (1.98 mL) under N₂ atmosphere. The reaction mixture was stirred at 70° C. for 4 h in a sealed tube. The solvent was evaporated in vacuo and the crude mixture was purified by flash column chromatography (SiO₂, MeOH in DCM, gradient from 0/100 to 5/95). A second purification was performed by RP HPLC (stationary phase: C18 XBridge 30×100 mm 5 um), mobile phase: [0.1% NH₄CO₃H/NH₄OH pH 9 solution in water]/CH₃CN, gradient from 54/46 to 64/36). The desired fractions were collected and concentrated in vacuo to give product 16 (43 mg, 76%) as an oil.

### E15. Preparation of Product 17, 18 and 19

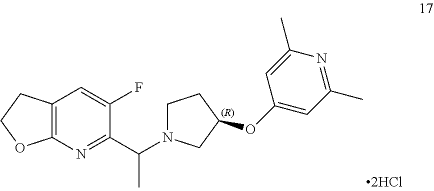

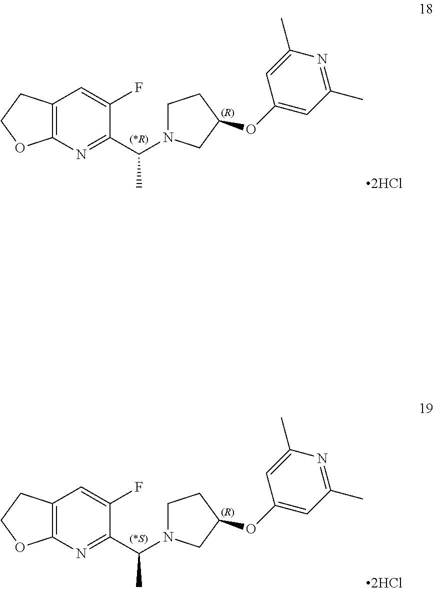

A solution of intermediate 63 (173 mg, 0.86 mmol) in anhydrous CH₃CN (10.8 mL) was added to a stirred mixture of intermediate 21 (150 mg, 0.78 mmol) and K₂CO₃ (323 mg, 2.34 mmol) in a sealed tube. The reaction mixture was stirred at 70° C. for 36 h. The reaction mixture was diluted with water and extracted with EtOAc. The organic layer was dried (Na₂SO₄), filtered and the solvents were evaporated in vacuo. The crude product was purified by flash column chromatography (silica, MeOH in DCM, gradient from 0/100 to 5/95). The desired fractions were collected and the solvents were evaporated in vacuo. The product was triturated with DIPE and filtered to afford a solid (102 mg, 37%).

The solid (23 mg) was dissolved in Et₂O (3 mL) and HCl (1M in Et₂O, 0.1 mL, 0.1 mmol) was added at 0° C. The mixture was stirred for 30 min. The solid was filtered off and washed with cold Et₂O to afford product 17 (20 mg) as a white solid.

The other fraction of the solid (89 mg) was purified via chiral SFC (stationary phase: Chiralpak IG 5 μm 250*20 mm, mobile phase: 75% CO₂, 25% MeOH (0.3% i-PrNH₂)) to give fraction A (20 mg) and fraction B (29 mg).

Fraction A (20 mg) was dissolved in Et₂O (2 mL) and HCl (2M in Et₂O, 0.06 mL, 0.12 mmol) was added at 0° C. The mixture was stirred for 30 min at room temperature. The solid was filtered to afford product 18 (22.5 mg) as a cream solid.

Product 19 (33.5 mg) was obtained following an analogous procedure using fraction B (29 mg) as starting material.

### E16. Preparation of Product 20

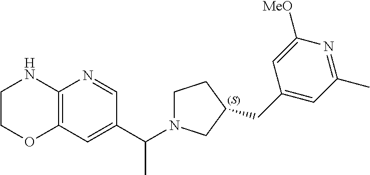

Intermediate 61 (160 mg, 0.60 mmol) and Ti(Oi-Pr)₄ (CAS: 546-68-9; 0.43 mL, 1.45 mmol) were added to a stirred solution of intermediate 26 (100 mg, 0.49 mmol) in DCM (1.98 mL) at room temperature and under N₂ atmosphere. The reaction mixture was stirred for 16 h. The mixture was cooled at 0° C. and THF (0.57 mL) was added, followed by methylmagnesium bromide (1.4M in THF and toluene, 1.73 mL, 2.42 mmol) dropwise. The reaction mixture was stirred at this temperature for 25 min and at room temperature for 18 h. The mixture was treated with NH₄Cl (sat. solution) and DCM, filtered through Celite® and the filtrate was extracted with DCM. The combined organic layers were dried (Na₂SO₄), filtered and the solvent was evaporated in vacuo. The crude mixture was purified by flash column chromatography (SiO₂, MeOH in EtOAc, gradient from 0/100 to 12/88). The desired fractions were collected and concentrated in vacuo to afford product 20 (28.8 mg, 16%) as a brown sticky solid.

### E17. Preparation of Products 21, 22 and 23

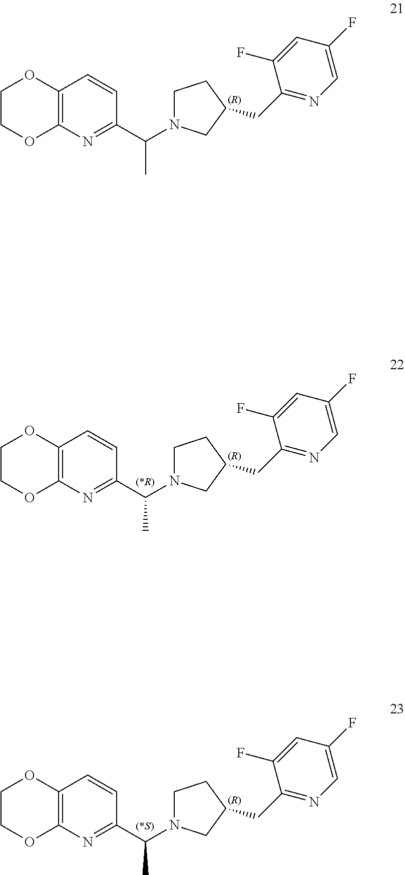

Products 21, 22 and 23 were prepared following an analogous procedure to the one described for the synthesis of product 20 using intermediate 35 and 2,3-dihydro-[1,4]dioxino[2,3-b]pyridine-6-carbaldehyde (CAS: 615568-24-6) as starting materials. The crude product was purified by flash column chromatography (SiO₂, MeOH in EtOAc, gradient from 0/100 to 10/90). The desired fractions were collected and concentrated in vacuo. A second purification was performed by flash column chromatography (silica, MeOH in DCM, gradient from 0/100 to 10/90). The desired fractions were collected and evaporated in vacuo to yield product 21 (118.9 mg, 65%) as a pale yellow oil.

A purification was performed via chiral SFC (stationary phase: CHIRACEL OJ-H 5 μm 250*20 mm, mobile phase: 75% CO₂, 25% i-PrOH (0.3% i-PrNH₂)) to afford product 22 (45 mg, 25%) and product 23 (42 mg, 23%). The two products were further purified by preparative LC (stationary phase: irregular bare silica 40 g, mobile phase: 0.5% NH₄OH, 92% DCM, 8% MeOH) to give product 22 (40 mg, 22%) as a pale yellow oil and product 23 (38 mg, 21%) as a pale yellow oil.

### E18. Preparation of Products 24, 25 and 26

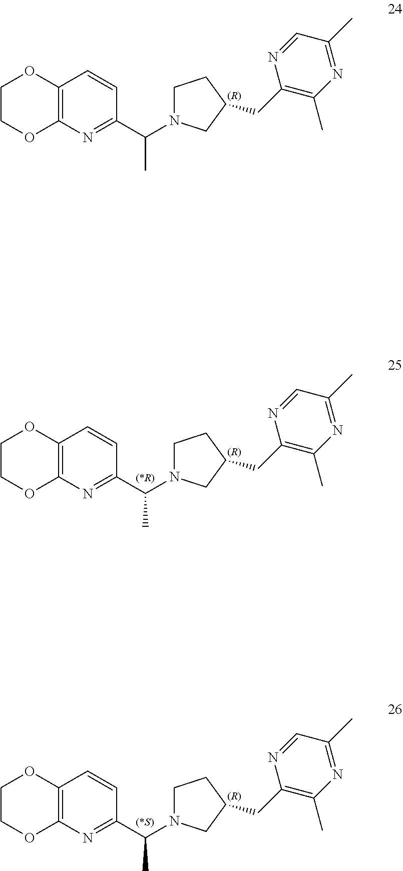

Products 24, 25 and 26 were prepared following an analogous procedure to the one described for the synthesis of product 20 using intermediate 29 and 2,3-dihydro-[1,4]dioxino[2,3-b]pyridine-6-carbaldehyde (CAS: 615568-24-6) as starting materials. The crude product was purified by flash column chromatography (SiO₂, MeOH in EtOAc, gradient from 0/100 to 15/85). The desired fractions were collected and concentrated in vacuo to yield product 24 (128.5 mg, 69%) as a light brown oil.

A purification was performed via chiral SFC (stationary phase: CHIRACEL OJ-H 5 μm 250*20 mm, mobile phase: 70% CO₂, 30% i-PrOH (0.3% i-PrNH₂)) to give product 25 (48 mg, 26%) and product 26 (48 mg, 26%) as a light brown oils.

### E19. Preparation of Products 27, 28 and 29

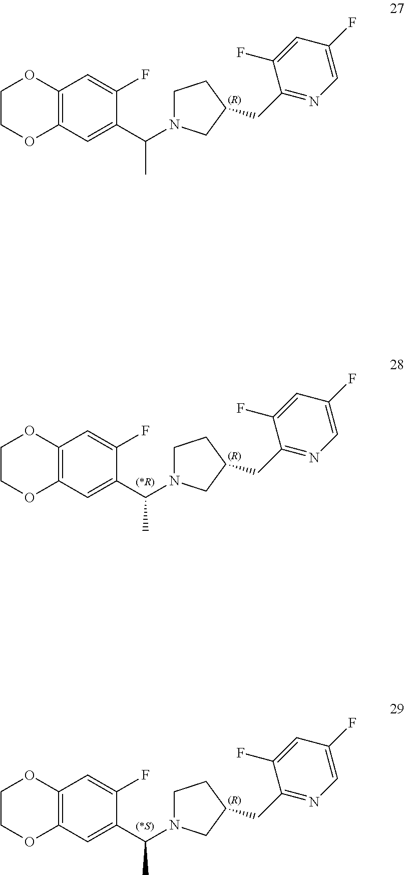

Ti(Oi-Pr)₄ (CAS: 546-68-9; 0.12 mL, 0.41 mmol) and sodium cyanoborohydride (CAS: 25895-60-7; 30.9 mg, 0.49 mmol) were added sequentially to a mixture of intermediate 35 (100 mg, 0.51 mmol) and intermediate 86 (80.3 mg, 0.41 mmol) in DCE (1.66 mL) in a sealed tube at room temperature. The reaction mixture was stirred at 80° C. for 16 h. The mixture was treated with NaHCO₃ (sat. solution), diluted with DCM and filtered through Celite®. The organic layer separated, dried (Na₂SO₄), filtered and the solvent was evaporated in vacuo. The crude product was purified by flash column chromatography (silica, EtOAc in DCM, gradient from 0/100 to 100/0). The desired fractions were collected and evaporated in vacuo to yield product 27 (80.9 mg, 52%) as a colorless oil.

A purification was performed via chiral SFC (stationary phase: CHIRALPAK AD-H 5 μm 250*30 mm, mobile phase: 88% CO₂, 12% EtOH (0.3% i-PrNH₂)) to afford product 28 (23 mg, 15%) and product 29 (26 mg, 17%) as colorless oils.

### E20. Preparation of Products 30, 31 and 32

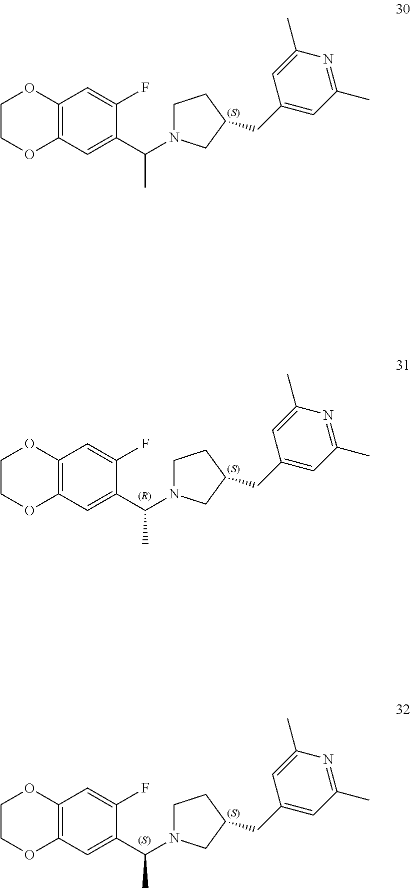

To a solution of intermediate 25 (100 mg, 0.53 mmol) in DCE (2.08 mL) were added intermediate 86 (124 mg, 0.63 mmol) and Ti(Oi-Pr)₄ (CAS: 546-68-9; 0.23 mL, 0.79 mmol). The reaction mixture was stirred at 80° C. for 20 h. Sodium cyanoborohydride (46.2 mg, 0.74 mmol) was added and the reaction mixture was stirred at 80° C. for 1 h and cooled to room temperature for 3 h. NH₄Cl (sat. solution) was added and the product extracted with DCM. The organic layer was dried (MgSO₄), filtered and the solvents were evaporated in vacuo. The crude product was purified by flash column chromatography (silica, NH₃ (7M in MeOH) in DCM, gradient from 0/100 to 10/90). A second purification was performed by RP HPLC (stationary phase: C18 XBridge 30×100 mm 5 μm), mobile phase: NH₄HCO₃ (0.25% solution in water)/CH₃CN, gradient from 60/40 to 43/57). The desired fractions were collected and concentrated in vacuo. The fractions were dissolved in EtOAc, washed with NaHCO₃ (sat. solution), dried (Na₂SO₄), filtered and concentrated in vacuo to give product 30 (10.5 mg, 5%), product 31 (13.7 mg, 7%) and product 32 (15.4 mg, 8%) as orange oils.

### E21. Preparation of Products 33 and 34

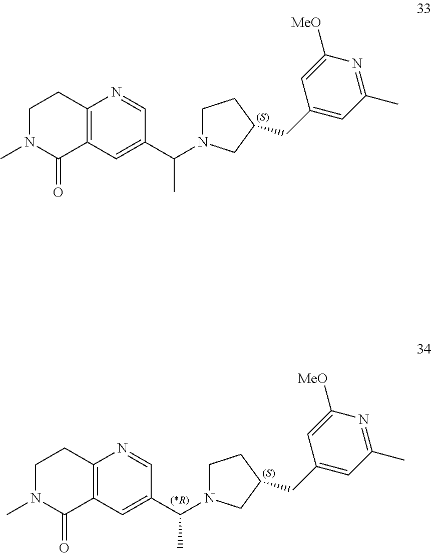

Intermediate 67 (96.8 mg, 0.51 mmol) and Ti(Oi-Pr)₄ (CAS: 546-68-9; 0.22 mL, 0.73 mmol) were added to a solution of intermediate 26 (100 mg, 0.49 mmol) in DCE (1.94 mL). The reaction mixture was stirred at 80° C. for 16 h, cooled to room temperature and methylmagnesium bromide (1.4M solution, 1.73 mL, 2.42 mmol) was added. The reaction mixture was stirred overnight. NaHCO₃ (sat. solution) was added and the mixture was extracted with DCM. The organic layer was dried (MgSO₄), filtered and the solvents were evaporated in vacuo. The crude product was purified by flash column chromatography (silica, NH₃ (7M in MeOH) in DCM, gradient from 0/100 to 10/90). A second purification was performed by RP HPLC (stationary phase: C18 XBridge 30×100 mm 5 μm), mobile phase: NH₄HCO₃ (0.25% solution in water)/CH₃CN, gradient from 75/25 to 57/43). The desired fractions were collected and concentrated in vacuo. The residue was dissolved in EtOAc, washed with NaHCO₃ (sat. solution), dried (Na₂SO₄), filtered and concentrated in vacuo to give product 33 (40 mg, 21%) and product 34 (23.2 mg, 12%) as colorless oils.

### E22. Preparation of Products 35 and 36

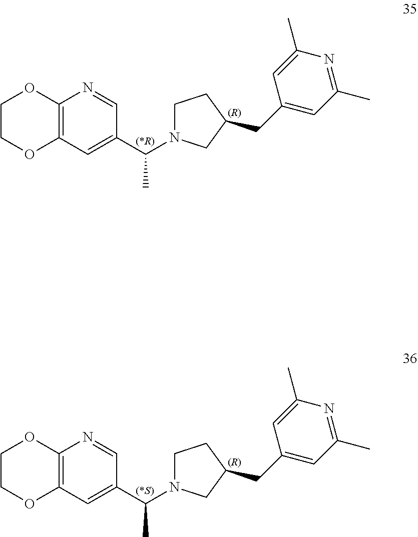

Intermediate 120 (100 mg, 0.50 mmol) was dissolved in CH₃CN (4 mL) and intermediate 24 (105 mg, 0.55 mmol) and K₂CO₃ (208 mg, 1.50 mmol) were added. The reaction mixture was stirred overnight at 80° C. Water was added and the mixture was extracted with DCM. The combined organic extracts were dried (Na₂SO₄), filtered and evaporated in vacuo. The crude product was purified by flash column chromatography (silica, NH₃ (7N in MeOH) in DCM, gradient from 0/100 to 10/90) (twice). Another purification was performed by RP HPLC (stationary phase: C18 XBridge 30×100 mm 5 μm), mobile phase: NH₄HCO₃ (0.25% solution in water)/CH₃CN, gradient from 80/20 to 0/100). The desired fractions were collected and concentrated in vacuo. The residue was dissolved in EtOAc and washed with NaHCO₃ (sat. solution). The organic layer was dried (Na₂SO₄), filtered and concentrated in vacuo to yield a racemic mixture (32.8 mg, 19%), product 35 (30.3 mg, 17%) and product 36 (24 mg, 14%) as colorless oils.

### E23. Preparation of Product 37

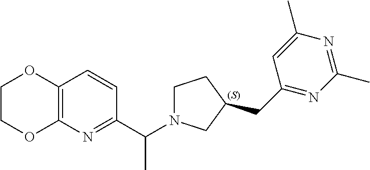

Product 37 was prepared following an analogous procedure to the one described for the synthesis of products 35 and 36 using intermediate 107 and intermediate 31 as starting materials.

The crude product was purified by flash column chromatography (silica, NH₃ (7M in MeOH) in DCM, gradient from 0/100 to 10/90) (twice). The desired fractions were collected and concentrated in vacuo to afford product 37 (137.2 mg, 97%) as a light brown oil.

### E24. Preparation of Products 38, 39, 40 and 41

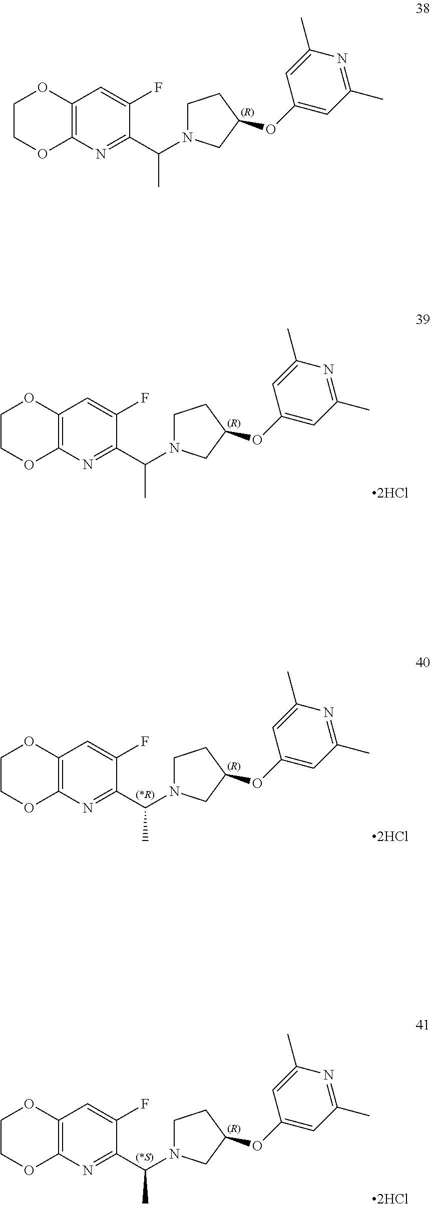

Products 38, 39, 40 and 41 were prepared following an analogous procedure to the one described for the synthesis of products 35 and 36 using intermediate 129 and intermediate 21 as starting materials.

The crude mixture was purified by flash column chromatography (silica, NH₃ (7N in MeOH) in DCM, gradient from 0/100 to 10/90). The desired fractions were collected and concentrated in vacuo. A second purification was performed by RP HPLC (stationary phase: C18 XBridge 30×100 mm 5 μm), mobile phase: NH₄HCO₃ (0.25% solution in water)/CH₃CN, gradient from 75/25 to 57/43). The desired fractions were collected and concentrated in vacuo. The residue was dissolved in EtOAc and washed with NaHCO₃ (sat. solution). The organic layer was dried (Na₂SO₄), filtered and concentrated in vacuo to afford product 38 (170.9 mg, 55%).

HCl (6M in i-PrOH, 44.6 μL, 0.27 mmol) was added to a stirred solution of product 38 (20 mg, 53.6 μmop in Et₂O (0.1 mL). The mixture was stirred at room temperature for 4 h and the solvent was concentrated in vacuo. tert-Butyl methyl ether was added and the mixture was sonicated for 5 min. The solvent was concentrated in vacuo. The process was repeated until the obtention of a solid which was dried under vacuum to afford product 139 (15.2 mg, 64%) as a light brown solid.

A purification was performed on product 138 via chiral SFC (stationary phase: Chiralpak IC 5 μm 250*21.2 mm, mobile phase: 78% CO₂, 22% EtOH (0.3% i-PrNH₂)) to give fraction A (75 mg) and fraction B (47 mg). Fraction B was purified again via chiral SFC (stationary phase: Chiralpak IC 5 μm 250*21.2 mm, mobile phase: 78% CO₂, 22% EtOH (0.3% i-PrNH₂)) to give 40 mg of fraction B.

Fraction A was dissolved in Et₂O (0.2 mL) and 7N HCl-IPA (0.2 mL) was added. The mixture was stirred at room temperature for 4 h. The solvent was concentrated in vacuo to afford product 40 (80 mg) as a cream solid. Fraction B was also converted into product 41 (86 mg) following an analogous procedure.

### E25. Preparation of Products 42 and 43

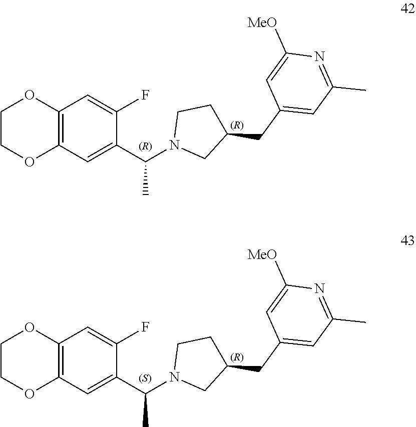

Intermediate 86 (99.8 mg, 0.51 mmol) and Ti(Oi-Pr)₄ (CAS: 546-68-9; 0.22 mL, 0.73 mmol) were added to a solution of intermediate 4 (100 mg, 0.49 mmol) in DCE (2 mL). The reaction mixture was stirred at 80° C. for 4 h, cooled to room temperature and sodium cyanoborohydride (CAS: 25895-60-7; 36.6 mg, 0.58 mmol) was added. The reaction mixture was stirred for 16 h. The reaction was quenched with NaHCO₃ (sat. solution) and diluted with DCM. The emulsion was filtered through a pad of Celite®. The filtrate was extracted with DCM. The combined organic layers were dried (MgSO₄), filtered and the solvents were evaporated in vacuo. The crude product was purified by flash column chromatography (silica, EtOAc in heptane, gradient from 30/70 to 70/30). The desired fractions were collected and concentrated in vacuo to afford a mixture of products (85 mg) as a colorless oil. The mixture was purified by RP HPLC (stationary phase: C18 XBridge 30×100 mm 5 μm), mobile phase: NH₄HCO₃ (0.25% solution in water)/CH₃CN, gradient from 47/53 to 30/70). The desired fractions were collected and concentrated in vacuo to give fractions A (35 mg) and fractions B (30 mg) as oils. Fractions A and B were diluted with DCM and NaHCO₃ (solution).

The aqueous phases were extracted with DCM. The organic layers were dried (MgSO₄), filtered and the solvents were evaporated in vacuo to give product 42 (30 mg, 16%) and product 43 (30 mg, 16%).

### E26. Preparation of Products 44 and 45

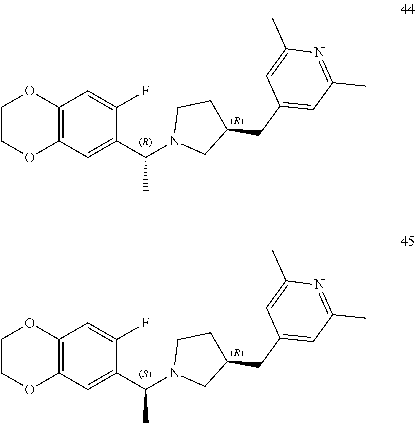

Products 44 and 45 were prepared following an analogous procedure to the one described for the synthesis of products 42 and 43 using intermediate 6 and intermediate 86 as starting materials.

The crude product was purified by flash column chromatography (silica, MeOH in DCM, gradient from 0/100 to 5/95). The desired fractions were collected and concentrated in vacuo to afford a mixture of products (140 mg) as a colorless oil. The mixture was purified by RP HPLC (stationary phase: C18 XBridge 30×100 mm 5 μm), mobile phase: NH₄HCO₃ (0.25% solution in water)/CH₃CN, gradient from 60/40 to 43/57) to afford fraction A and fraction B. Fractions A and B were diluted with DCM and NaHCO₃ (solution). The aqueous phases were extracted with EtOAc. The organic layers were dried (MgSO₄), filtered and the solvents were evaporated in vacuo to give product 44 (21 mg, 11%) and product 45 (20 mg, 10%) as oils.

### E27. Preparation of Product 46

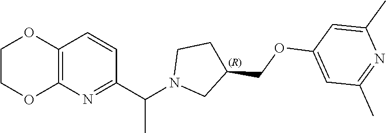

K₂CO₃ (208 mg, 1.50 mmol) was added to a mixture of intermediate 107 (100 mg, 0.50 mmol) and intermediate 118 (114 mg, 0.55 mmol) in CH₃CN (4 mL). The reaction mixture was stirred for 20 h at 60° C. The reaction mixture was diluted with EtOAc, filtered through Celite®, washed with EtOAc and the solvents were evaporated in vacuo. The crude product was purified by flash column chromatography (silica, NH₃ (7M in MeOH) in DCM, gradient from 0/100 to 5/95). The desired fractions were collected and concentrated in vacuo to afford product 46 (130 mg, 70%) as an oil.

### E28. Preparation of Product 47

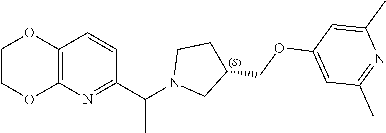

Product 47 was prepared following an analogous procedure to the one described for the synthesis of product 46 using intermediate 107 and intermediate 8 as starting materials. The crude product was purified by flash column chromatography (silica, NH₃ (7M in MeOH) in DCM, gradient from 0/100 to 05/95). The desired fractions were collected and concentrated in vacuo to give product 47 (170 mg, 92%) as an oil.

### E29. Preparation of Product 48

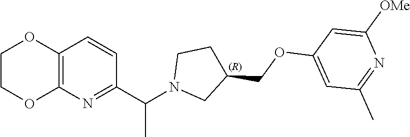

Product 48 was prepared following an analogous procedure to the one described for the synthesis of product 46 using intermediate 107 and intermediate 2 as starting materials.

The crude product was purified by flash column chromatography (silica, NH₃ (7M in MeOH) in DCM, gradient from 0/100 to 4/96). The desired fractions were collected and concentrated in vacuo to yield product 48 (190 mg, 73%) as an oil.

### E30. Preparation of Product 49

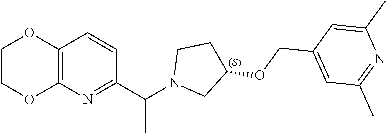

Product 49 was prepared following an analogous procedure to the one described for the synthesis of product 46 using intermediate 107 and intermediate 127 as starting materials.

The crude product was purified by flash column chromatography (silica, NH₃ (7M in MeOH) in DCM, gradient from 0/100 to 04/96). A second purification was performed by RP HPLC (stationary phase: C18 XBridge 30×100 mm 5 μm), mobile phase: NH₄HCO₃ (0.25% solution in water)/CH₃CN, gradient from 80/20 to 60/40). The desired fractions were collected and concentrated in vacuo to afford product 49 (70 mg, 65%) as an oil.

### E31. Preparation of Product 50

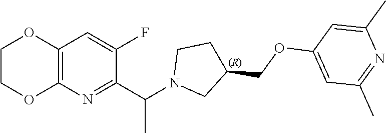

Product 50 was prepared following an analogous procedure to the one described for the synthesis of product 46 using intermediate 129 and intermediate 147 as starting materials.

The crude product was purified by flash column chromatography (silica, NH₃ (7M in MeOH) in DCM, gradient from 0/100 to 04/96). The desired fractions were collected and concentrated in vacuo to afford product 50 (70 mg, 75%) as an oil.

### E32. Preparation of Product 51 and 52

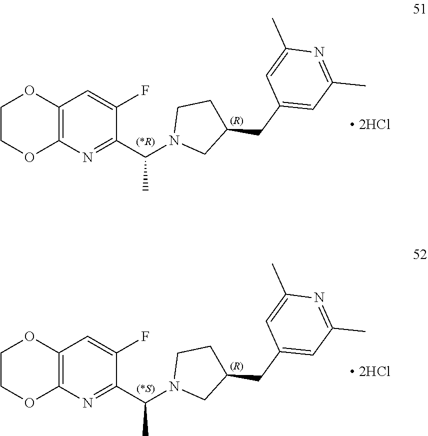

Products 51 and 52 were prepared following an analogous procedure to the one described for the synthesis of product 46 using intermediate 79 and intermediate 24 as starting materials.

The crude product was purified by flash column chromatography (silica, NH₃ (7M in MeOH) in DCM, gradient from 0/100 to 07/93). The desired fractions were collected and concentrated in vacuo to afford a mixture of products (160 mg) as an oil. A purification was performed via chiral SFC (stationary phase: CHIRACEL OJ-H 5 μm 250*20 mm, mobile phase: 85% CO2, 15% EtOH (0.3% iPrNH2)) to give fraction A (52 mg) and fraction B (46 mg).

Fraction A (35 mg) was purified by flash column chromatography (silica, NH₃ (7M in MeOH) in DCM, gradient from 0/100 to 03/97). The desired fractions were collected and concentrated in vacuo. The resulting product was dissolved in tert-butyl methyl ether (2 mL) and HCl (2M in Et₂O, 2 mL, 4 mmol) was added under stirring. The precipitate was filtrated and dried at 50° C. under vacuum to afford product 51 (35 mg). Product 52 was prepared following an analogous procedure using fraction B as starting material.

### E33. Preparation of Product 53

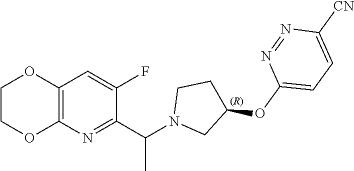

Product 53 was prepared following an analogous procedure to the one described for the synthesis of product 46 using intermediate 129 and intermediate 43 as starting materials.

The crude product was purified by flash column chromatography (silica, MeOH in DCM, gradient from 0/100 to 10/90). The desired fractions were collected and concentrated in vacuo to afford product 53 (69.5 mg, 71%).

### E34. Preparation of Products 54 and 55

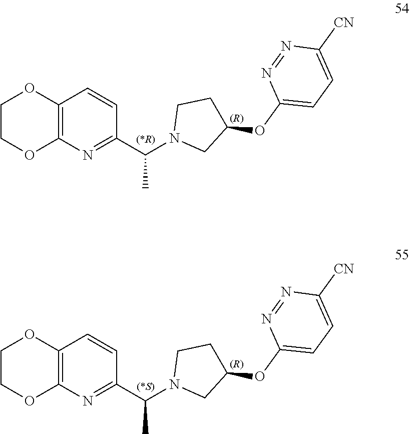

Products 54 and 55 were prepared following an analogous procedure to the one described for the synthesis of product 46 using intermediate 107 and intermediate 43 as starting materials.

The crude product was purified by flash column chromatography (silica, MeOH in DCM, gradient from 0/100 to 10/90) to afford a mixture of products. The mixture was purified by RP HPLC (stationary phase: C18 XBridge 30×100 mm 5 μm), mobile phase: NH₄HCO₃ (0.25% solution in water)/CH₃CN, gradient from 90/10 to 60/40) to afford product 54 (15.6 mg, 17%) and product 55 (16.4 mg, 18%).

### E35. Preparation of Product 56

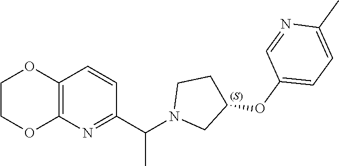

Product 56 was prepared following an analogous procedure to the one described for the synthesis of product 46 using intermediate 107 and intermediate 41 as starting materials.

The crude product was purified by flash column chromatography (silica, MeOH in DCM, gradient from 0/100 to 10/90). The desired fractions were collected and concentrated in vacuo to afford product 56 (17.5 mg, 27%).

### E36. Preparation of Product 57

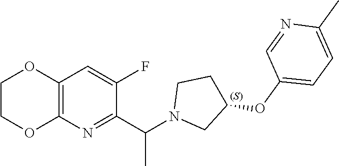

Product 57 was prepared following an analogous procedure to the one described for the synthesis of product 46 using intermediate 129 and intermediate 41 as starting materials.

The crude product was purified by flash column chromatography (silica, MeOH in DCM, gradient from 0/100 to 10/90). The desired fractions were collected and concentrated in vacuo to afford product 57 (31.2 mg, 60%) as a colorless oil.

### E37. Preparation of Product 58

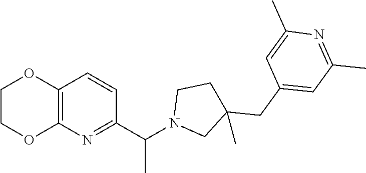 [SMILES: Cc1cc(CC2(C)CCN(C(C)c3ccc4c(n3)OCCO4)C2)cc(C)n1]

K₂CO₃ (779 mg, 5.64 mmol) was added to a stirred mixture of intermediate 37 (384 mg, 1.88 mmol) and intermediate 107 (338 mg, 1.69 mmol) in anhydrous CH₃CN (14.8 mL). The reaction mixture was stirred at 80° C. in a sealed tube for 12 h. The reaction mixture was diluted with water and extracted with EtOAc. The organic layer was dried (Na₂SO₄), filtered and the solvents were evaporated in vacuo. The residue was purified by flash column chromatography (SiO₂, EtOAc in heptane, gradient from 0/100 to 100/0). The desired fractions were collected and evaporated in vacuo to give product 58 (502 mg, 73%) as a pale brown oil.

### E38. Preparation of Product 59

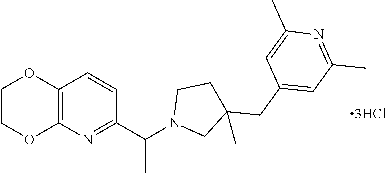 [SMILES: Cc1cc(CC2(C)CCN(C(C)c3ccc4c(n3)OCCO4)C2)cc(C)n1]

Product 58 (435 mg) was suspended in Et₂O and treated with HCl (2N in Et₂O, 4 eq) at room temperature. The white precipitate was filtered and dried to give product 59 (428.7 mg) as a white solid.

### E39. Preparation of Product 60

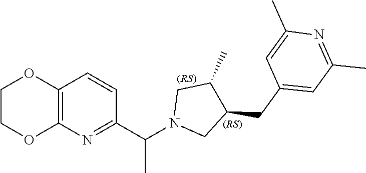 [SMILES: Cc1cc(C[C@H]2CN(C(C)c3ccc4c(n3)OCCO4)C[C@@H]2C)cc(C)n1]

Product 60 was prepared following an analogous procedure to the one described for the synthesis of product 58 using intermediate 39 and intermediate 107 as starting materials. The residue was purified by RP HPLC (stationary phase: C18 XBridge 30×100 mm 5 μm), mobile phase: NH₄HCO₃ (0.25% solution in water)/CH₃CN, gradient from 80/20 to 0/100). The desired fractions were collected and solvents were evaporated in vacuo to afford product 60 (125.8 mg, 56%) as a colorless oil.

### E40. Preparation of Product 61

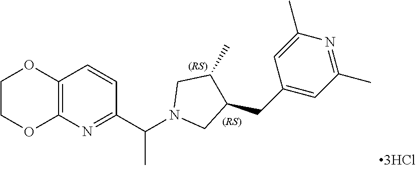 [SMILES: Cc1cc(C[C@H]2CN(C(C)c3ccc4c(n3)OCCO4)C[C@@H]2C)cc(C)n1]

Product 61 was prepared following an analogous procedure to the one described for the synthesis of compound 59 using product 60 as starting material.

### E41. Preparation of Product 62

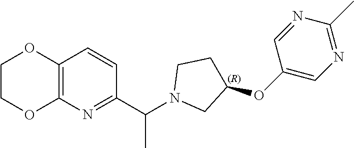

Product 62 was prepared following an analogous procedure to the one described for the synthesis of product 58 using intermediate 33 and intermediate 107 as starting materials.

The crude product was purified by flash column chromatography (silica, MeOH in DCM, gradient from 0/100 to 10/90). The desired fractions were collected and concentrated in vacuo. A second purification was performed by RP HPLC (stationary phase: C18 XBridge 30×100 mm 5 um), mobile phase: [0.1% NH₄CO₃H/NH₄OH pH 9 solution in water]/CH₃CN, gradient from 67/33 to 50/50). The desired fractions were collected and concentrated in vacuo to afford product 62 (115 mg, 60%) as a light yellow solid.

### E42. Preparation of Products 63 and 64

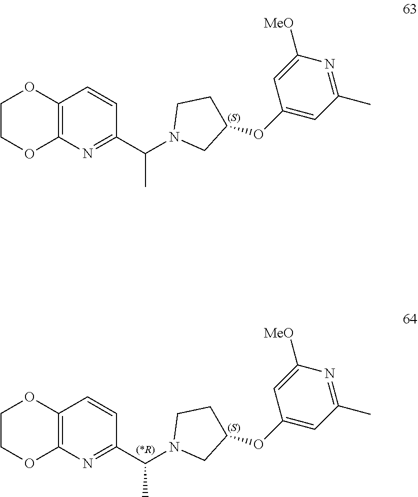

2,3-Dihydro-[1,4]dioxino[2,3-b]pyridine-6-carbaldehyde (CAS: 615568-24-6; 95.2 mg, 0.58 mmol) and Ti(Oi-Pr)₄ (CAS: 546-68-9; 0.21 mL, 0.72 mmol) were added to a stirred solution of intermediate 149 (100 mg, 0.48 mmol) in anhydrous DCM (1.92 mL). The reaction mixture was stirred at room temperature for 20 h. The reaction mixture was cooled to 0° C. and methylmagnesium bromide (1.4M in THF, 1.72 mL, 2.40 mmol) was added dropwise. The reaction mixture was stirred at 0° C. for 5 min and at room temperature for 2 h. NH₄Cl (sat. solution) was added and the product extracted with DCM. The organic layer was dried (MgSO₄), filtered and the solvents were evaporated in vacuo. The crude product was purified by flash column chromatography (silica, NH₃ (7M in MeOH) in DCM, gradient from 0/100 to 3/97). The residue was purified by RP HPLC (stationary phase: C18 X Bridge 30×100 mm 5 μm), mobile phase: NH₄HCO₃ (0.25% solution in water)/CH₃CN, gradient from 67/33 to 50/50). NaHCO₃ (sat. solution) was added and the product was extracted with DCM. The organic layer was dried (MgSO₄), filtered and the solvents were evaporated in vacuo to afford product 63 (15 mg, 8%) and product 64 (20 mg, 11%) as yellow oils.

### E48. Preparation of Products 70, 71 and 72

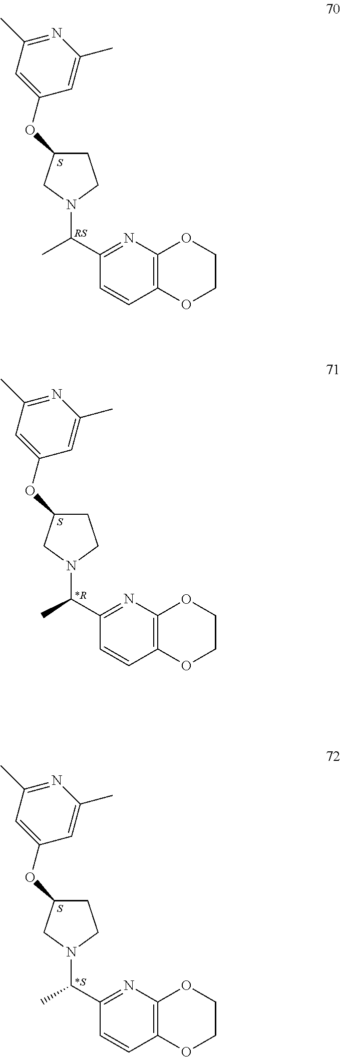

Product 70 was prepared following an analogous procedure to the one described for the synthesis of product 1 using intermediate 20 (100 mg, 0.52 mmol) and 2,3-dihydro[1,4]dioxino[2,3-b]pyridine-6-carbaldehyde (CAS: 615568-24-6) as starting materials. Crude product 70 was purified by RP HPLC (stationary phase: C18 XBridge 30×100 mm 5 μm, mobile phase: gradient from 54% NH₄HCO₃ 0.25% solution in water, 46% CH₃CN to 36% NH₄HCO₃ 0.25% solution in water, 64% CH₃CN). The desired fractions were collected and concentrated in vacuo. The residue thus obtained was dissolved in EtOAc and washed with an aq sat sol of NaHCO₃. The organic phases were separated, dried (Na₂SO₄), filtered and concentrated in vacuo to yield product 70 (121 mg, 65%, mixture of diastereoisomers) as a colorless oil.

Product 70 (110 mg) was purified via chiral SFC (stationary phase: CHIRACEL OJ-H 5 μm 250*20 mm, mobile phase: 80% CO₂, 20% MeOH (0.3% iPrNH₂)) yielding product 71 (50 mg, 27%) and product 72 (42 mg, 23%) both as oils. Product 71 was taken up in diethyl ether and treated with HCl (6N solution in i-PrOH). The solvents were evaporated in vacuo to yield product 71 (60.3 mg, 27%, 2×HCl salt) as a cream color solid. Product 72 was taken up in diethyl ether and treated with HCl (6N solution in i-PrOH). The solvents were evaporated in vacuo to yield product 72 (49 mg, 22%, 2×HCl salt) as a cream color solid.

### E49. Preparation of Products 73, 74 and 75

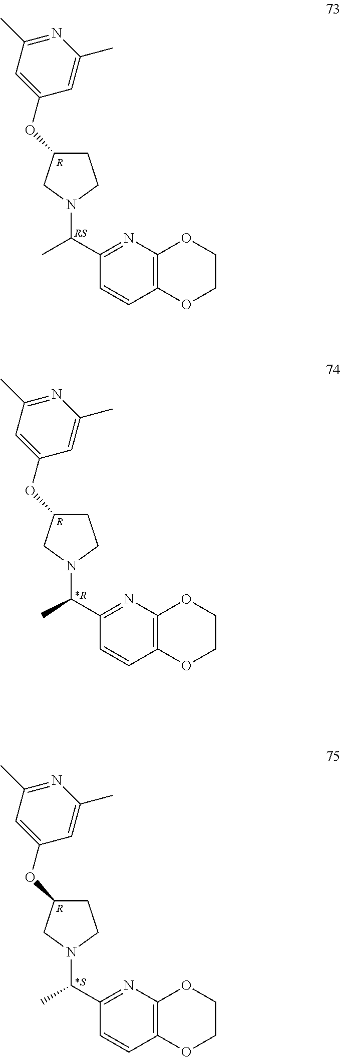

Product 73 was prepared following an analogous procedure to the one described for the synthesis of product 1 using intermediate 21 (100 mg, 0.52 mmol) and 2,3-dihydro[1,4]dioxino[2,3-b]pyridine-6-carbaldehyde (CAS: 615568-24-6) as starting materials. Crude product 73 was purified by RP HPLC (stationary phase: C18 XBridge 30×100 mm 5 μm, mobile phase: gradient from 80% 10 mM NH₄HCO₃/NH₄OH pH=9 solution in Water, 20% CH₃CN to 60% 10 mM NH₄HCO₃/NH₄OH pH=9 solution in water, 40% CH₃CN). The desired fractions were collected and concentrated in vacuo. The residue thus obtained was dissolved in EtOAc and washed with an aq sat sol of NaHCO₃. The organic phases were separated, dried (Na₂SO₄), filtered and concentrated in vacuo to yield product 73 (78 mg, 42%, mixture of diastereoisomers) as a colorless oil.

Product 73 (65 mg) was purified via chiral SFC (stationary phase: CHIRALPAK AD-H 5 μm 250*30 mm, mobile phase: 80% CO₂, 20% MeOH (0.3% iPrNH₂)) yielding product 74 (20 mg, 11%) and product 75 (19 mg, 10%) both as oils.

### E50. Preparation of Product 76

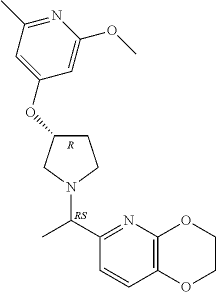

Product 76 was prepared following an analogous procedure to the one described for the synthesis of product 1 using intermediate 22 (100 mg, 0.48 mmol) and 2,3-dihydro[1,4]dioxino[2,3-b]pyridine-6-carbaldehyde (CAS: 615568-24-6) as starting materials. Product 76 was purified by RP HPLC (stationary phase: C18 XBridge 30×100 mm 5 μm, mobile phase: gradient from 67% NH₄HCO₃ 0.25% solution in water, 33% CH₃CN to 50% NH₄HCO₃ 0.25% solution in water, 50% CH₃CN). The desired fractions were collected and concentrated in vacuo yielding product 76 (61.1 mg, 34%, mixture of diastereoisomers) as a colorless oil.

### E51. Preparation of Products 77, 100, 101, 102 and 103

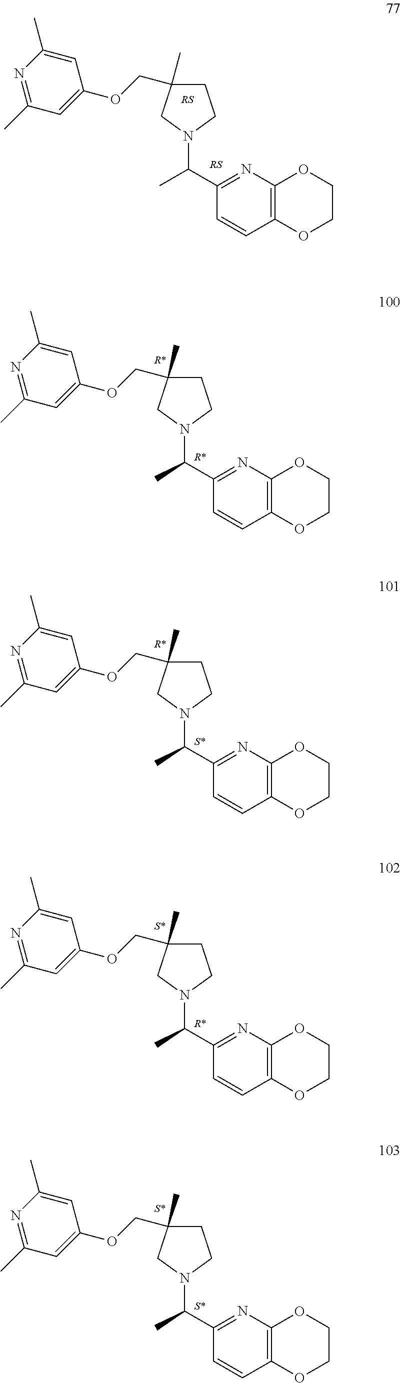 [SMILES: Cc1cc(OCC2(C)CCN(C(C)c3ccc4c(n3)OCCO4)C2)cc(C)n1 ; Cc1cc(OC[C@]2(C)CCN([C@H](C)c3ccc4c(n3)OCCO4)C2)cc(C)n1 ; Cc1cc(OC[C@]2(C)CCN([C@H](C)c3ccc4c(n3)OCCO4)C2)cc(C)n1 ; Cc1cc(OC[C@]2(C)CCN([C@H](C)c3ccc4c(n3)OCCO4)C2)cc(C)n1 ; Cc1cc(OC[C@]2(C)CCN([C@H](C)c3ccc4c(n3)OCCO4)C2)cc(C)n1]

Product 77 was prepared following an analogous procedure to the one described for the synthesis of product 1 using intermediate 23 (336 mg, 1.52 mmol) and 2,3-dihydro[1,4]dioxino[2,3-b]pyridine-6-carbaldehyde (CAS: 615568-24-6) as starting materials. Crude product 77 was purified by RP HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and concentrated in vacuo to yield product 77 (232 mg, 40%, mixture of diastereoisomers) as a colorless oil.

Product 77 (220 mg) was purified via chiral SFC (stationary phase: Lux-Cellulose-4 5 μm 250*21.2 mm, mobile phase: 80% CO₂, 20% EtOH (0.3% iPrNH₂)) yielding product 77 (101 mg), product 102 (55 mg, 9%) and product 103 (49 mg, 8%) all as oils. Product 77 (101 mg) was purified via chiral SFC (stationary phase: CHIRALPAK AD-H 5 μm 250*30 mm, mobile phase: 90% CO₂, 10% iPrOH (0.3% iPrNH₂)) yielding product 100 (44 mg, 7%) and impure product 101 (47 mg, 8%) all as oils.

Impure product 101 (47 mg) was purified via chiral SFC (stationary phase: CHIRACEL OJ-H 5 μm 250*20 mm, mobile phase: 75% CO₂, 25% iPrOH (0.3% iPrNH₂)) yielding product 101 (39 mg, 7%) as an oil.

Product 100 was suspended in Et₂O and treated with HCl (4 equiv, 2N solution in Et₂O) at room temperature. The pale brown precipitate was filtered and dried in the oven to yield product 100 (40 mg, 5%, 3×HCl salt) as a white solid.

Product 101 was suspended in Et₂O and treated with HCl (4 equiv, 2N solution in Et₂O) at room temperature. The pale brown precipitate was filtered and dried in the oven to yield product 101 (36 mg, 5%, 3×HCl salt) as a white solid.

Product 103 was suspended in Et₂O and treated with HCl (4 equiv, 2N solution in Et₂O) at room temperature. The pale brown precipitate was filtered and dried in the oven to yield product 103 (48 mg, 6%, 3×HCl salt) as a white solid.

### E52. Preparation of Products 78, 104 and 105

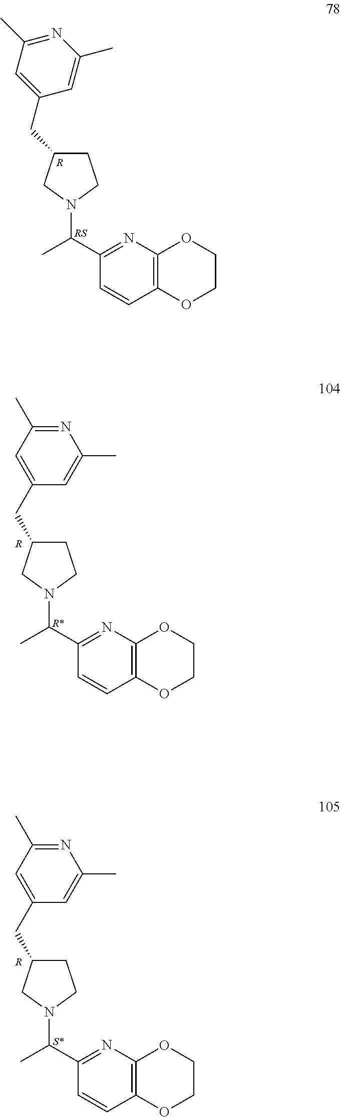

Product 78 was prepared following an analogous procedure to the one described for the synthesis of product 1 using intermediate 24 (100 mg, 0.53 mmol) and 2,3-dihydro[1,4]dioxino[2,3-b]pyridine-6-carbaldehyde (CAS: 615568-24-6) as starting materials. Product 78 (120 mg, 64%, mixture of diastereoisomers) was isolated as a colorless oil.

Product 78 (110 mg) was purified via chiral SFC (stationary phase: CHIRALPAK AD-H 5 μm 250*30 mm, mobile phase: 90% CO₂, 10% EtOH (0.3% iPrNH₂)) yielding product 104 (37 mg, 20%) and impure product 105 (41 mg, 22%) all as oils. Product 104 (37 mg) was dissolved in Et₂O (1 mL) and then HCl (1 mL, 2N in Et₂O) was added. The resulting solid was filtered and dried to give product 104 (35 mg, 16%, 2×HCl salt) as a sticky foam. Impure product 105 (41 mg) was taken up in DCM and washed with NaHCO₃ (aq. sat. soltn.). The organic layer was separated, dried (MgSO₄), filtered and the solvents evaporated in vacuo to give impure product 105 (40 mg) which was further purified by flash column chromatography (silica; 7M ammonia solution in methanol in DCM 0/100 to 05/95). The desired fractions were collected and concentrated in vacuo to yield pure product 105 (30 mg, 16%) as an oil. Product 105 (30 mg) was dissolved in Et₂O (1 mL) and then HCl (1 mL, 2N in Et₂O) was added. The resulting solid was filtered and dried to give product 105 (30 mg, 13%, 2×HCl salt) as a sticky foam.

### E53. Preparation of Product 79

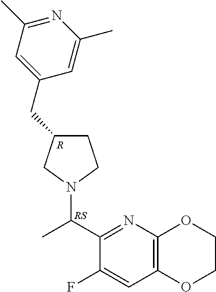

Product 79 was prepared following an analogous procedure to the one described for the synthesis of product 2 using intermediate 24 (106 mg, 0.55 mmol) and intermediate 86 as starting materials. Product 79 was purified by RP HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and concentrated in vacuo yielding impure product 79 (12 mg, 6%, mixture of diastereoisomers) as a colorless oil. Impure product 79 (12 mg) was further purified by flash column chromatography (silica; 7M ammonia solution in methanol in DCM 0/100 to 2/98). The desired fractions were collected and concentrated in vacuo to give product 79 (8.5 mg, 4%, mixture of diastereoisomers) as an oil.

### E54. Preparation of Product 80

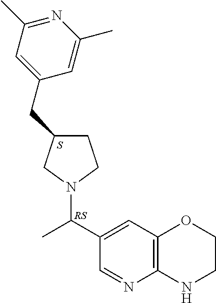

Product 80 was prepared following an analogous procedure to the one described for the synthesis of product 14 using intermediate 96 (80 mg, 0.18 mmol) as starting material. Crude product 80 was purified by RP HPLC (stationary phase: C18 XBridge 30×100 mm 5 μm, mobile phase: gradient from 47% 10 mM NH₄HCO₃/NH₄OH pH=9 solution in water, 53% MeOH to 24% 10 mM NH₄HCO₃/NH₄OH pH=9 solution in water, 76% MeOH). The desired fractions were collected and concentrated in vacuo to yield product 80 (20 mg, 31%, mixture of diastereoisomers)

### E55. Preparation of Products 81 and 82

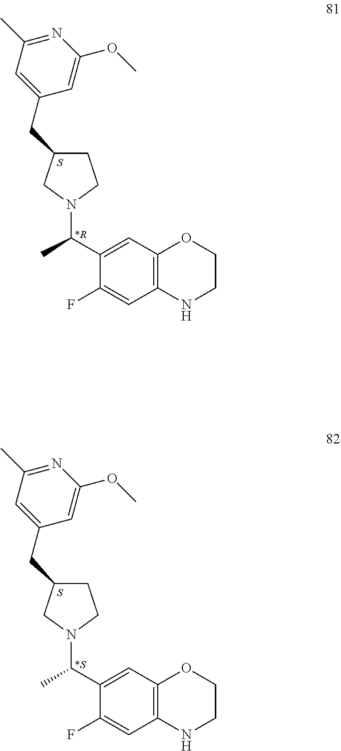

Product 81 and product 82 were prepared following an analogous procedure to the one described for the synthesis of product 1 using intermediate 26 (200 mg, 0.53 mmol) and intermediate 123 (300 mg, 1.07 mmol) as starting materials. A mixture (258 mg) of crude Product 81 and crude product 82 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and concentrated in vacuo yielding a mixture (215 mg) of product 81 and product 82 which was further purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and concentrated in vacuo yielding product 81 (46 mg, 12%, single racemic diastereoisomer) and product 82 (40 mg, 11%, single racemic diastereoisomer) both as a yellow oil.

### E56 Preparation of Products 83, 84 and 85

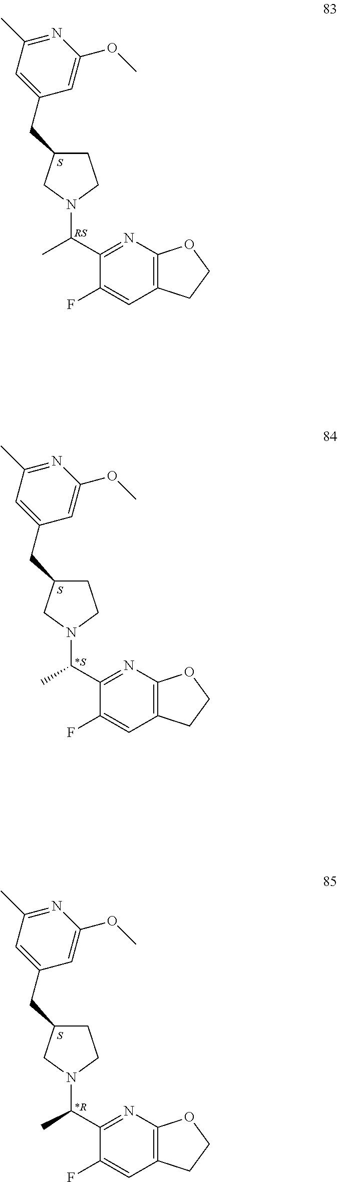

Product 83 was prepared following an analogous procedure to the one described for the synthesis of product 2 using intermediate 26 (50 mg, 0.242 mmol) and intermediate 97 as starting materials. Product 83 was purified by RP HPLC (stationary phase: C18 XBridge 30×100 mm 5 μm, mobile phase: gradient from 54% 0.1% NH₄CO₃H/NH₄OH pH 9 solution in water, 46% CH₃CN to 64% 0.1% NH₄CO₃H/NH₄OH pH 9 solution in water, 36% CH₃CN), the desired fractions were collected and concentrated in vacuo to get yielding product 83 (31.2 mg, 33%, mixture of diastereoisomers) as a colorless oil.

Product 83 (23 mg) was purified via chiral SFC (stationary phase: CHIRACEL OJ-H 5 μm 250*20 mm, mobile phase: 90% CO₂, 10% iPrOH (0.3% iPrNH₂)) yielding product 84 (10 mg, 11%) and product 85 (11 mg, 12%) as oils.

### E57. Preparation of Products 86, 87 and 88

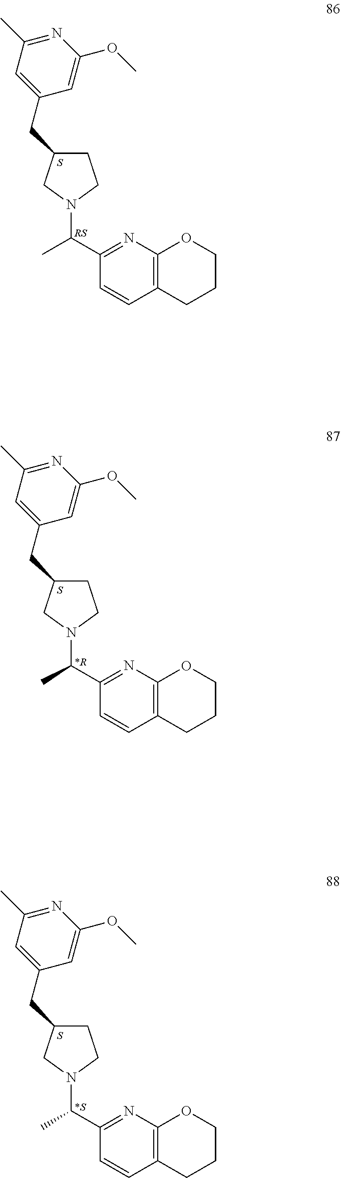

Product 86 was prepared following an analogous procedure to the one described for the synthesis of product 2 using intermediate 26 (150 mg, 0.727 mmol) and 7-acetyl(3,4-dihydro-2H-pyrano)[2,3-b]pyridine (CAS: 253874-77-0) as starting materials. Product 86 was purified by RP HPLC (Stationary phase: C18 XBridge 30×100 mm 5 μm, mobile phase: gradient from 54% 0.1% NH₄CO₃H/NH₄OH pH 9 solution in water, 46% CH₃CN to 64% 0.1% NH₄CO₃H/NH₄OH pH 9 solution in water, 36% CH₃CN), the desired fractions were collected and concentrated in vacuo to get yielding product 86 (90 mg, 34%) as mixture of isomers. The compound was dissolved in EtOAc and was treated with a saturated solution of NaHCO₃ (stirred 30 min), the organic layer was separated and evaporated in vacuo to afford product 86 (82.6 mg, 31%) as oil.

Product 86 (70 mg) was purified via chiral SFC (stationary phase: CHIRALPAK AD-H 5 μm 250*30 mm, mobile phase: 70% CO₂, 30% MeOH (0.3% iPrNH₂)) yielding impure product 87 (39 mg) and product 88 (25 mg, 9%) both as oils.

Impure product 87 (39 mg) was purified via preparative LC (stationary phase: irregular bare silica 40 g, mobile phase: 0.5% NH₄OH, 94% DCM, 6% MeOH) yielding product 87 (32 mg, 12%) as an oil.

### E58. Preparation of Products 89, 106 and 107

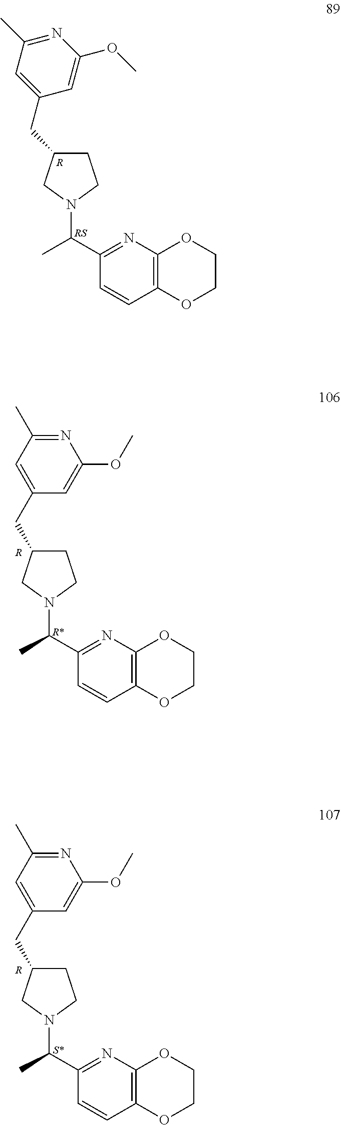

Product 89 was prepared following an analogous procedure to the one described for the synthesis of product 1 using intermediate 27 (100 mg, 0.485 mmol) and 2,3-dihydro[1,4]dioxino[2,3-b]pyridine-6-carbaldehyde (CAS: 615568-24-6) as starting materials. Product 89 (98 mg, 55%) was obtained as an oil.

Product 89 (85 mg) was purified via chiral SFC (stationary phase: CHIRALPAK AD-H 5 μm 250*30 mm, mobile phase: 90% CO₂, 10% EtOH (0.3% iPrNH₂)) yielding product 106 (33 mg, 18%) and product 107 (35 mg, 19%).

### E59. Preparation of Product 90

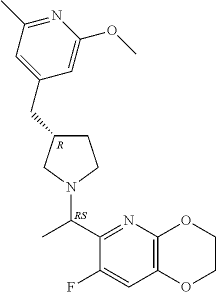

Product 90 was prepared following an analogous procedure to the one described for the synthesis of product 2 using intermediate 27 (115 mg, 0.558 mmol) and intermediate 100 (100 mg, 0.507 mmol) as starting materials. Product 90 was purified by RP HPLC (stationary phase: C18 XBridge 30×100 mm 5 μm, mobile phase: gradient from 67% NH₄HCO₃ 0.25% solution in water, 33% CH₃CN to 50% NH₄HCO₃ 0.25% solution in water, 50% CH₃CN). The desired fractions were collected and concentrated in vacuo to give impure product 90 (15 mg). Impure product 90 (15 mg) was purified by flash column chromatography (silica; 7M ammonia solution in methanol in DCM 0/100 to 2/98). The desired fractions were collected and concentrated in vacuo to give product 90 (9.5 mg, 5%) as an oil.

### E60. Preparation of Products 91, 92, 93, 94 and 95

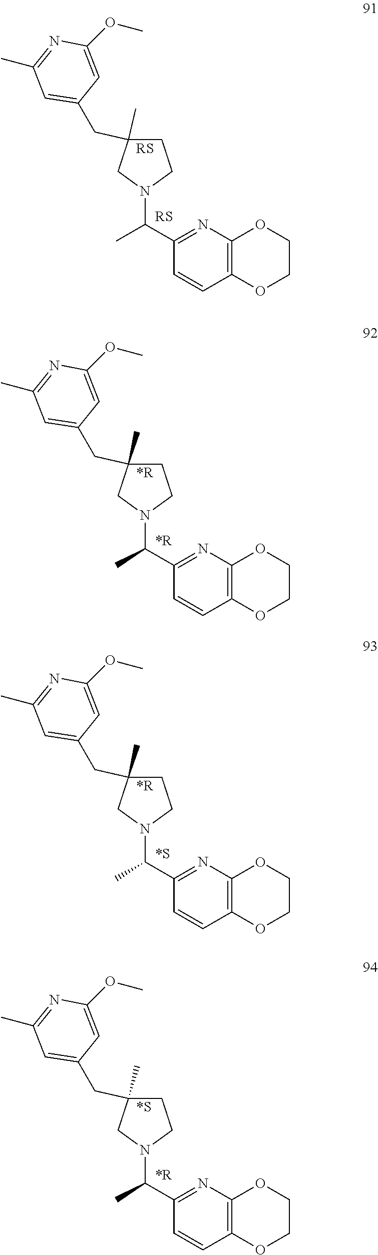 [SMILES: COc1cc(CC2(C)CCN(C(C)c3ccc4c(n3)OCCO4)C2)cc(C)n1 ; COc1cc(C[C@]2(C)CCN([C@H](C)c3ccc4c(n3)OCCO4)C2)cc(C)n1 ; COc1cc(C[C@]2(C)CCN([C@@H](C)c3ccc4c(n3)OCCO4)C2)cc(C)n1 ; COc1cc(C[C@@]2(C)CCN([C@H](C)c3ccc4c(n3)OCCO4)C2)cc(C)n1]

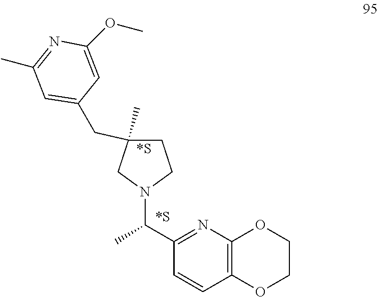 [SMILES: COc1cc(C[C@@]2(C)CCN([C@@H](C)c3ccc4c(n3)OCCO4)C2)cc(C)n1]

Product 91 was prepared following an analogous procedure to the one described for the synthesis of product 1 using intermediate 28 (154.8 mg, 0.726 mmol) and 2,3-dihydro[1,4]dioxino[2,3-b]pyridine-6-carbaldehyde (CAS: 615568-24-6) as starting materials. Crude product 91 was purified by RP HPLC (stationary phase: C18 XBridge 50×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and concentrated in vacuo to yield product 91 (151 mg, 56%, mixture of diastereoisomers) as a yellow oil.

Product 91 (140 mg) was purified via chiral SFC (stationary phase: CHIRALPAK AD-H 5 μm 250*30 mm, mobile phase: 92% CO₂, 8% iPrOH (0.9% iPrNH₂)) yielding product 91 (45 mg), product 92 (21 mg, 8%) and product 93 (21 mg, 8%) all as oils. Product 91 (45 mg) was purified via chiral SFC (stationary phase: CHIRACEL OJ-H 5 μm 250*20 mm, mobile phase: 92% CO₂, 8% MeOH (0.3% iPrNH₂)) yielding product 94 (17 mg, 6%) and impure product 95 (19 mg, 7%) all as oils.

### E61. Preparation of Products 96, 97 and 98

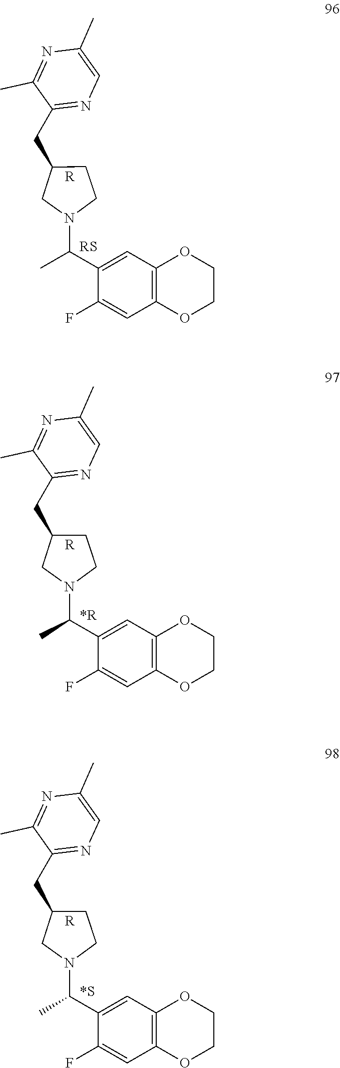

Product 96 was prepared following an analogous procedure to the one described for the synthesis of product 2 using intermediate 29 (100 mg, 0.523 mmol) and intermediate 86 (80.3 mg, 0.409 mmol) as starting materials. Product 96 (108.8 mg, 72%, mixture of diastereoisomers) was obtained as a colorless oil.

Product 96 (100 mg) was purified via SFC (stationary phase: CHIRALPAK AD-H 5 μm 250*30 mm, mobile phase: 85% CO₂, 15% EtOH (0.3% iPrNH₂)) yielding product 97 (42 mg, 28%) and product 98 (43 mg, 28%) as yellow oils.

### E68. Preparation of Product 115

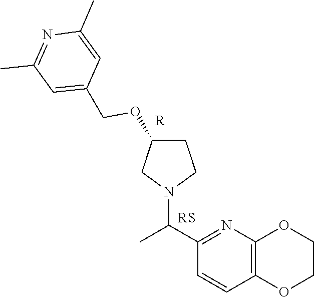

Product 115 was prepared following an analogous procedure to the one described for the synthesis of product 110 using intermediate 118 (110 mg, 0.53 mmol) and intermediate 107 (106 mg, 0.53 mmol) as starting materials.

### E69. Preparation of Product 116

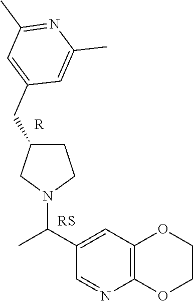

Product 116 was prepared following an analogous procedure to the one described for the synthesis of product 110 using intermediate 24 (104 mg, 0.55 mmol) and intermediate 120 (100 mg, 0.50 mmol) as starting materials.

### E70. Preparation of Product 117

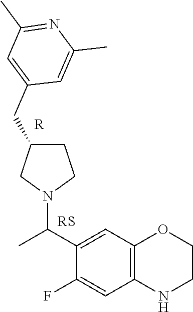

Trifluoroacetic acid (0.38 mL, 4.98 mmol) was added to a solution of intermediate 122 (130 mg, 0.28 mmol) in DCM (1.1 mL) at 0° C. The reaction mixture was stirred at rt for 18 h. as starting material. Then a NaHCO₃ (aq sat soltn) was added and the product was extracted with DCM. The organic layer was dried (MgSO₄), filtered and concentrated in vacuo. The crude product was purified by flash column chromatography (Silica, 7 M solution of ammonia in MeOH in DCM 0/100 to 30/70). The desired fractions were collected and evaporated in vacuo to give impure product 70. Impure product 70 was purified twice by RP HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected, a saturated solution of Na₂CO₃ was added and the product extracted with DCM. The organic phase was separated and the solvents evaporated in vacuo to yield product 117 (30 mg, 29%) as an oil.

### E71. Preparation of Product 118

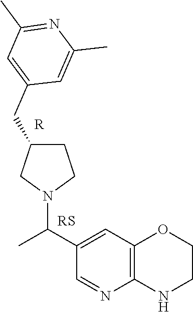

Product 118 was prepared following an analogous procedure to the one described for the synthesis of product 14 using intermediate 126 (30 mg, 0.068 mmol) as starting material. Crude product 118 was purified by RP HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected, a saturated solution of NaHCO₃ was added and the product extracted with DCM. The organic phase was separated and the solvents evaporated in vacuo to yield product 118 (22 mg, 91%) as an oil.

### E72. Preparation of Product 119

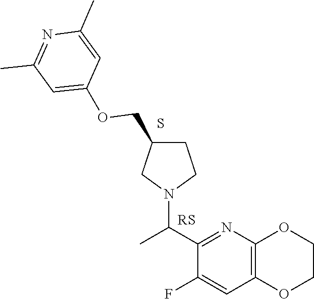

Product 119 was prepared following an analogous procedure to the one described for the synthesis of product 110 using intermediate 129 (50 mg, 0.23 mmol) and intermediate 127 (52 mg, 0.25 mmol) as starting materials. Product 119 was purified by RP HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and extracted with EtOAc. The organic phase was separated and the solvents evaporated in vacuo to yield product 119 (30 mg, 34%) as an oil.

### E73. Preparation of Products 120, 121, 122, 123 and 124

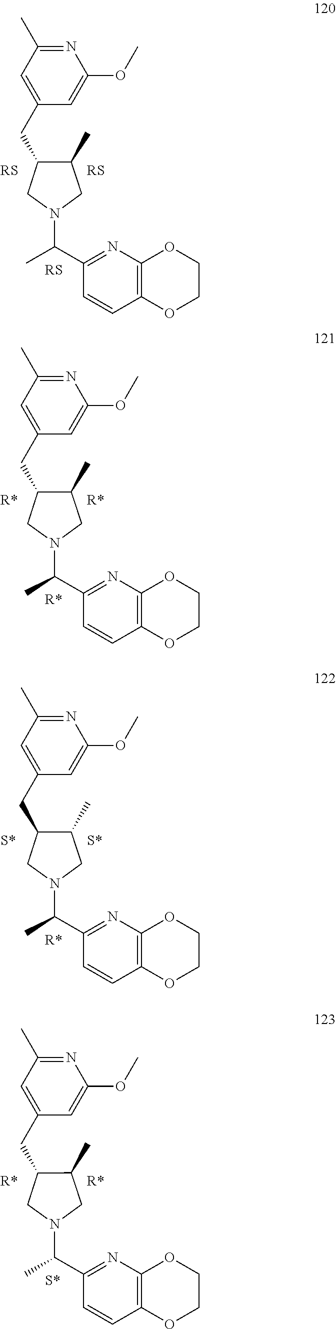 [SMILES: COc1cc(C[C@H]2CN(C(C)c3ccc4c(n3)OCCO4)C[C@@H]2C)cc(C)n1 ; COc1cc(C[C@H]2CN([C@H](C)c3ccc4c(n3)OCCO4)C[C@@H]2C)cc(C)n1 ; COc1cc(C[C@@H]2CN([C@H](C)c3ccc4c(n3)OCCO4)C[C@H]2C)cc(C)n1 ; COc1cc(C[C@H]2CN([C@@H](C)c3ccc4c(n3)OCCO4)C[C@@H]2C)cc(C)n1]

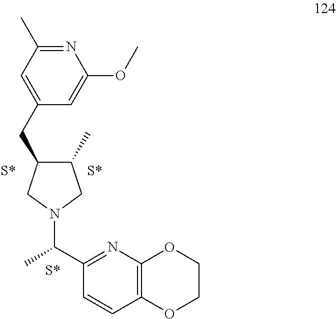 [SMILES: COc1cc(C[C@@H]2CN([C@@H](C)c3ccc4c(n3)OCCO4)C[C@H]2C)cc(C)n1]

Product 120 was prepared following an analogous procedure to the one described for the synthesis of product 1 using intermediate 131 (500 mg, 2.5 mmol) and 2,3-dihydro[1,4]dioxino[2,3-b]pyridine-6-carbaldehyde (CAS: 615568-24-6) as starting materials. Product 120 was purified by RP HPLC (stationary phase: XBridge C18 50×100 mm, 5 μm, mobile phase: gradient from 60% NH₄HCO₃ 0.25% solution in water, 40% CH₃CN to 43% NH₄HCO₃ 0.25% solution in water, 57% CH₃CN). The desired factions were evaporated in vacuo to yield product 120 (388 mg, 44%) as a sticky yellow oil.

Product 120 (375 mg) was purified via chiral SFC (stationary phase: CHIRACEL OJ-H 5 μm 250*30 mm, mobile phase: 75% CO₂, 25% iPrOH (0.3% iPrNH₂)) yielding product 121 (87 mg, 10%) a mixture (127 mg) of product 122 and product 123 and product 124 (88 mg, 10%).

The mixture (127 mg) of product 122 and product 123 was purified via chiral SFC (stationary phase: Chiralpak IC 5 μm 250*21.2 mm, mobile phase: 82% CO₂, 18% iPrOH (0.6% iPrNH₂)) yielding impure product 122 (50 mg) and product 123 (46 mg, 5%).

Impure product 122 (50 mg) was purified via preparative LC (stationary phase: irregular bare silica 10 g, mobile phase: 0.3% NH₄OH, 95% DCM, 5% MeOH) yielding product 122 (39 mg, 5%).

All products were obtained as sticky oils.

### E74. Preparation of Product 125

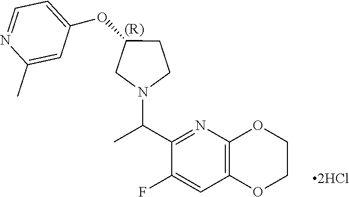

A mixture of intermediate 136.2HCl (191 mg, 0.76 mmol), intermediate 129 (165 mg, 0.76 mmol) and DIPEA (0.79 mL, 4.56 mmol) in anhydrous CH₃CN (2.92 mL) was stirred at 70° C. for 20 h. The reaction mixture was diluted with water and extracted with EtOAc. The organic layer was dried (Na₂SO₄), filtered and the solvents were evaporated in vacuo. The crude product was purified by flash column chromatography (silica, MeOH in DCM, gradient from 0/100 to 5/95). The desired fractions were collected and evaporated in vacuo.

The residue (177 mg) A was dissolved in Et₂O (1.25 mL) and HCl (2M in Et₂O, 0.74 mL, 1.48 mmol) was added under stirring. The precipitate was filtered and the product was dried under vacuum for 16 h at room temperature to give product 125 (145.3 mg, 44%) as a white solid.

### E75. Preparation of Product 126

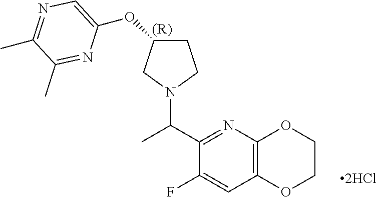

Product 126 was prepared following an analogous procedure to the one described for the synthesis of product 125 using intermediate 129 and intermediate 138 as starting materials.

### E76. Preparation of Product 127

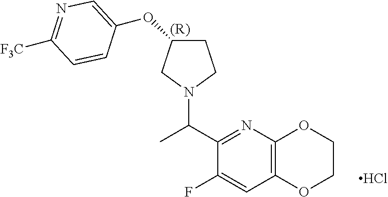

Product 127 was prepared following an analogous procedure to the one described for the synthesis of product 125 using intermediate 129 and intermediate 144 as starting materials.

### E77. Preparation of Product 128

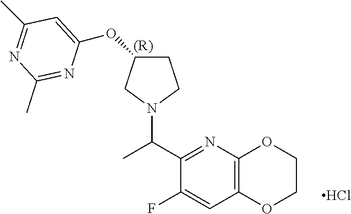

Product 128 was prepared following an analogous procedure to the one described for the synthesis of product 125 using intermediate 129 and intermediate 140.HCl as starting materials.

### E78. Preparation of Product 129

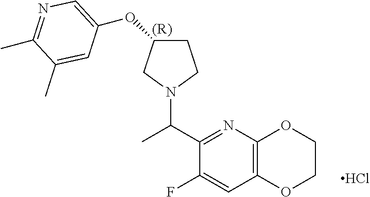

Product 129 was prepared following an analogous procedure to the one described for the synthesis of product 125 using intermediate 129 and intermediate 142.HCl as starting materials.

The following compounds were prepared following the methods exemplified in the Experimental Part. In case no salt form is indicated, the compound was obtained as a free base. ‘Ex. No.’ refers to the Example number according to which protocol the compound was synthesized. ‘Co. No.’ means compound number.

TABLE 1
(I)

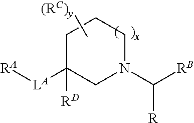

| Co. No. | Exp. No. | Co. Formula (I) | Salt Form |
|---|---|---|---|
| E1 | 1 | 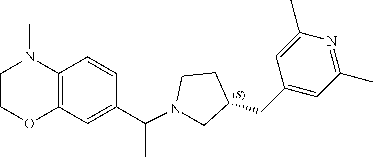 | 2HCl |
| E2 | 2 | 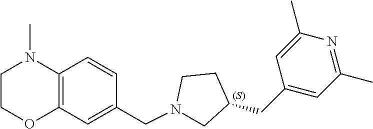 | 2HCl |
| E3 | 3 | 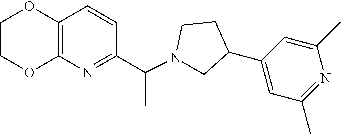 [SMILES: Cc1cc(C2CCN(C(C)c3ccc4c(n3)OCCO4)C2)cc(C)n1] | |
| E4 | 4 | 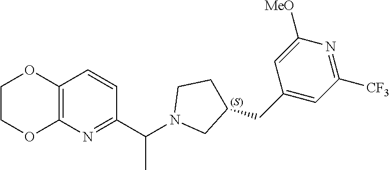 | |
| E5 | 5 | 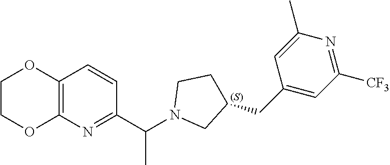 | |
| E6 | 6 | 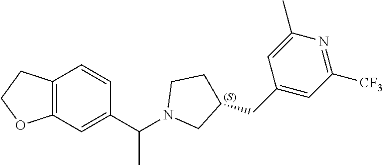 | |
| E7 | 7 | 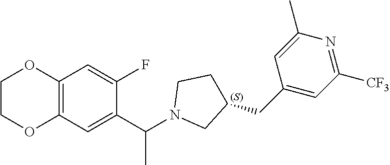 | |
| E8 | 8 | 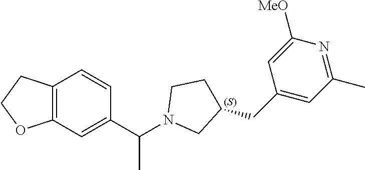 | |
| E9 | 9 | 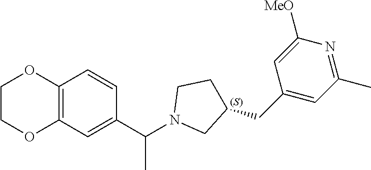 | |
| E10 | 10 | 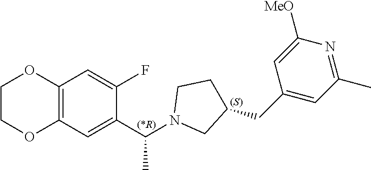 | |
| E10 | 11 | 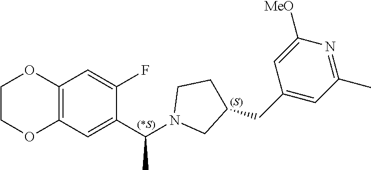 | |
| E11 | 12 | 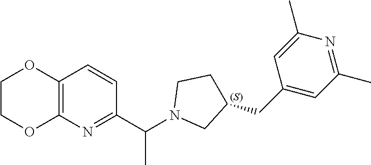 | |
| E12 | 13 | 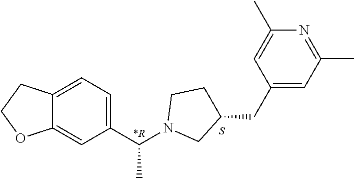 | |
| E12 | 14 | 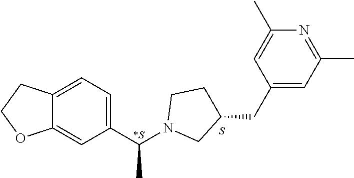 | |
| E13 | 15 | 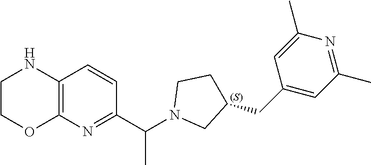 | |
| E14 | 16 | 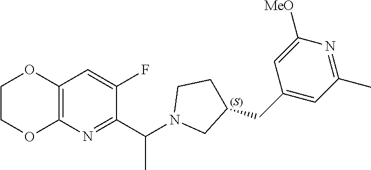 | |
| E15 | 17 | 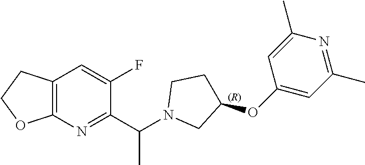 | 2HCl |
| E15 | 18 | 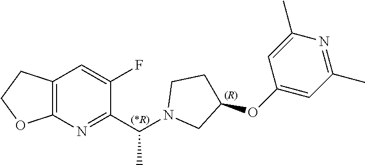 | 2HCl |
| E15 | 19 | 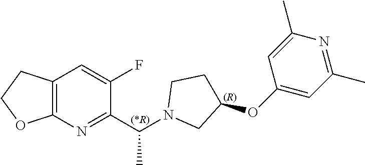 | 2HCl |
| E16 | 20 | 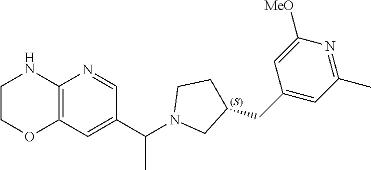 | |
| E17 | 21 | 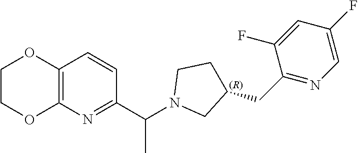 | |
| E17 | 22 | 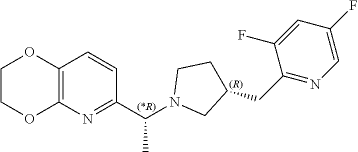 | |
| E17 | 23 | 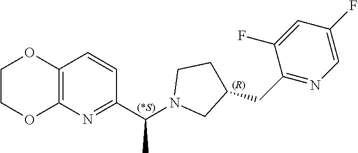 | |
| E18 | 24 | 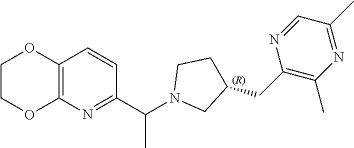 | |
| E18 | 25 | 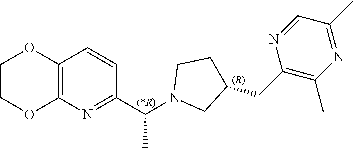 | |
| E18 | 26 | 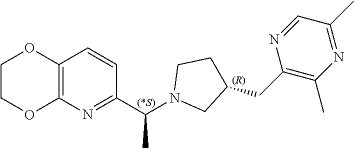 | |
| E19 | 27 | 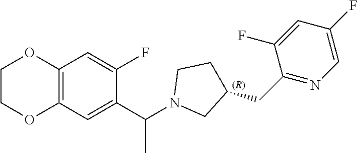 | |
| E19 | 28 | 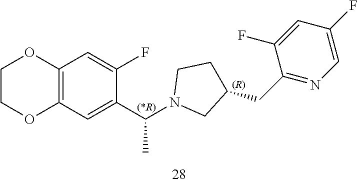 | |
| E19 | 29 | 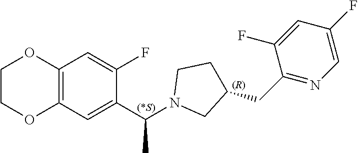 | |
| E20 | 30 | 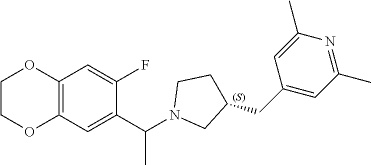 | |
| E20 | 31 | 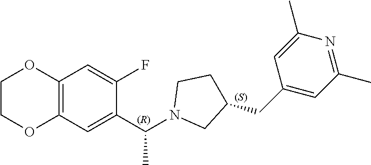 | |
| E20 | 32 | 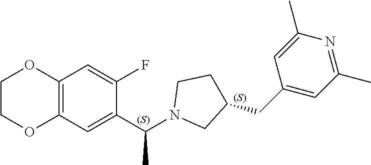 | |
| E21 | 33 | 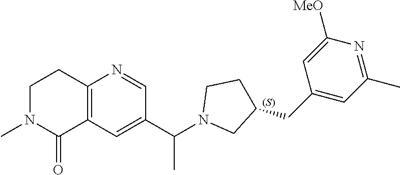 | |
| E21 | 34 | 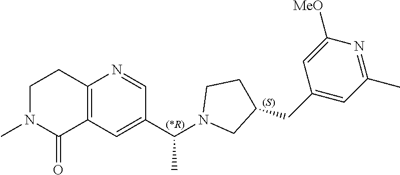 | |
| E22 | 35 | 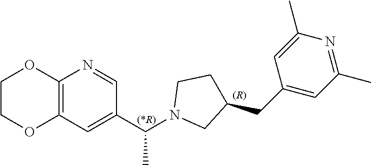 | |
| E22 | 36 | 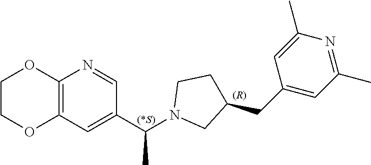 | |
| E23 | 37 | 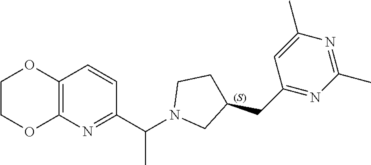 | |
| E24 | 38 | 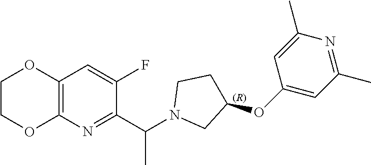 | |
| E24 | 39 | 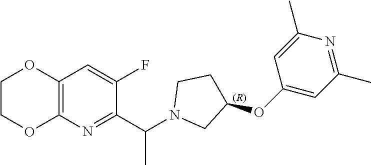 | 2HCl |
| E24 | 40 | 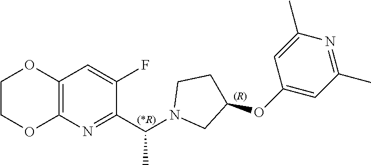 | 2HCl |
| E24 | 41 | 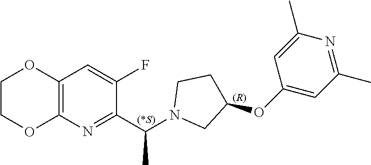 | 2HCl |
| E25 | 42 | 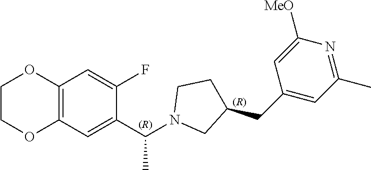 | |
| E25 | 43 | 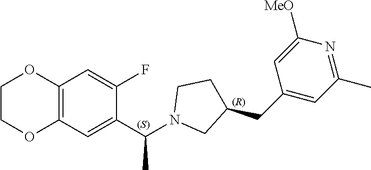 | |
| E26 | 44 | 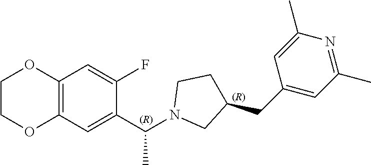 | |
| E26 | 45 | 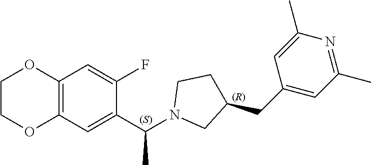 | |
| E27 | 46 | 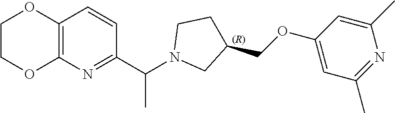 | |
| E28 | 47 | 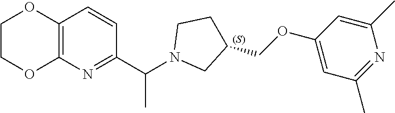 | |
| E29 | 48 | 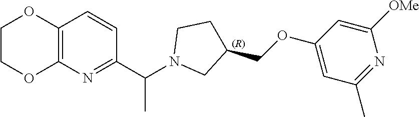 | |
| E30 | 49 | 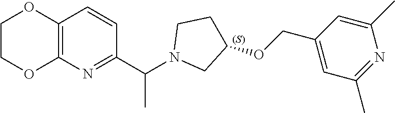 | |
| E31 | 50 | 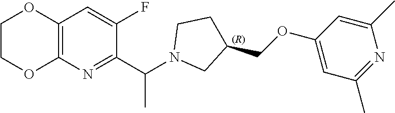 | |
| E32 | 51 | 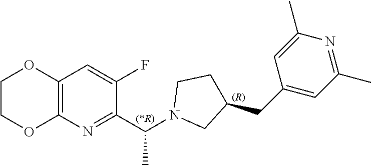 | 2HCl |
| E32 | 52 | 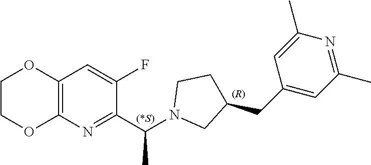 | 2HCl |
| E33 | 53 | 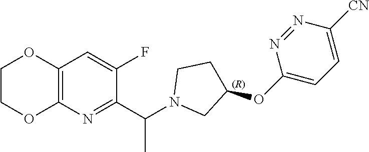 | |
| E34 | 54 | 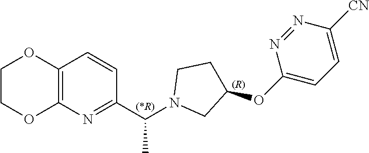 | |
| E34 | 55 | 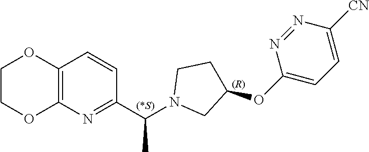 | |
| E35 | 56 | 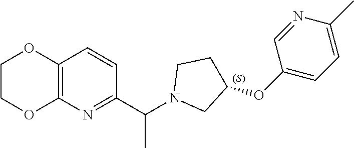 | |
| E36 | 57 | 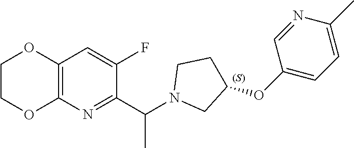 | |
| E37 | 58 | 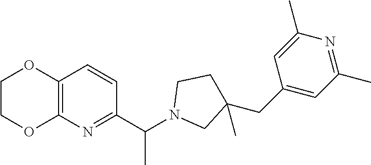 [SMILES: Cc1cc(CC2(C)CCN(C(C)c3ccc4c(n3)OCCO4)C2)cc(C)n1] | |
| E38 | 59 | 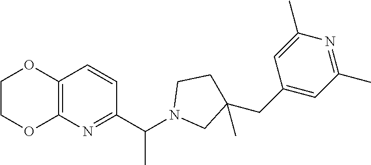 [SMILES: Cc1cc(CC2(C)CCN(C(C)c3ccc4c(n3)OCCO4)C2)cc(C)n1] | 3HCl |
| E39 | 60 | 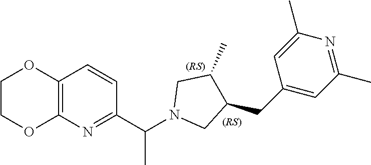 | |
| E40 | 61 | 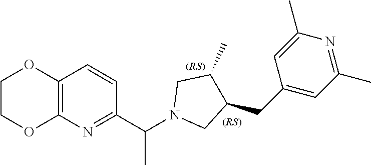 | 3HCl |
| E41 | 62 | 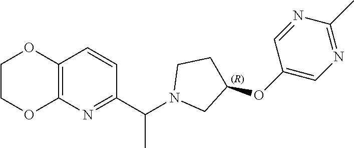 | |
| E42 | 63 | 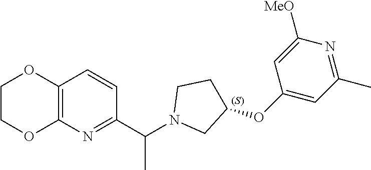 | |
| E42 | 64 | 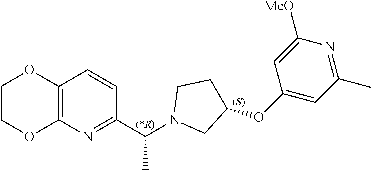 | |
| 70 | E48 | 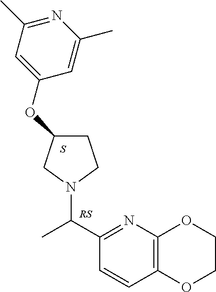 | |
| 71 | E48 | 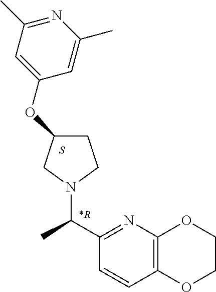 | •2HCl |
| 72 | E48 | 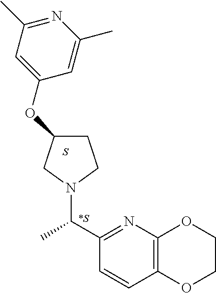 | •2HCl |
| 73 | E49 | 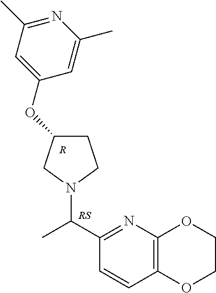 | |
| 74 | E49 | 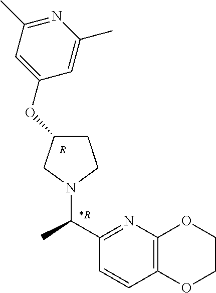 | |
| 75 | E49 | 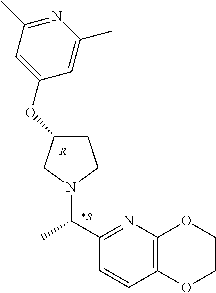 | |
| 76 | E50 | 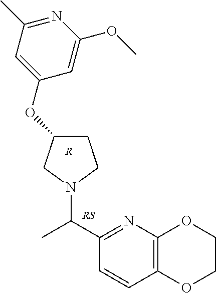 | |
| 77 | E51 | 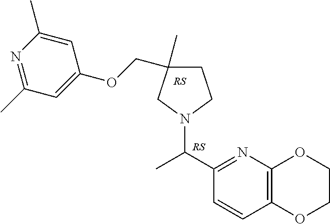 | |
| 78 | E52 | 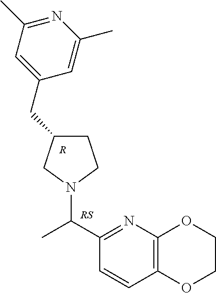 | |
| 79 | E53 | 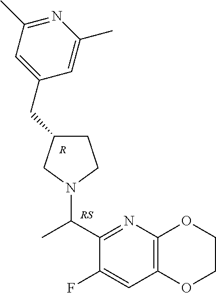 | |
| 80 | E54 | 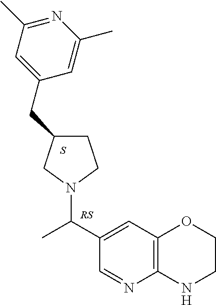 | |
| 81 | E55 | 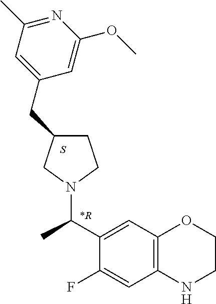 | |
| 82 | E55 | 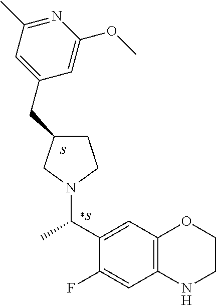 | |
| 83 | E56 | 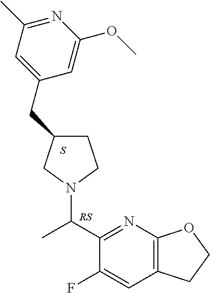 | |
| 84 | E56 | 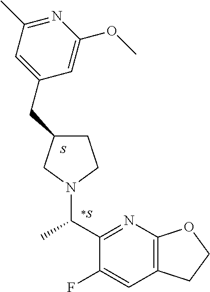 | |
| 85 | E56 | 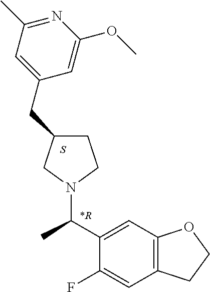 | |
| 86 | E57 | 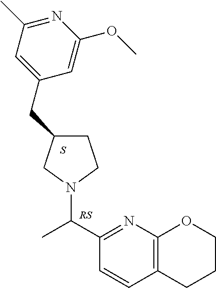 | |
| 87 | E57 | 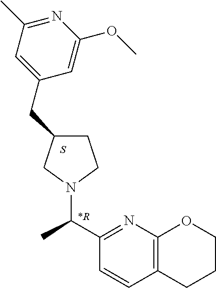 | |
| 88 | E57 | 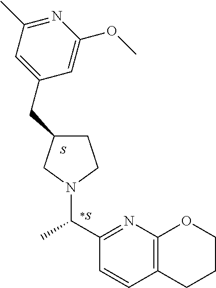 | |
| 89 | E58 | 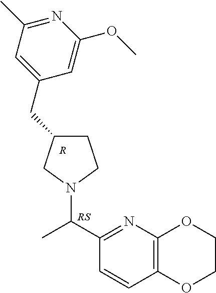 | |
| 90 | E59 | 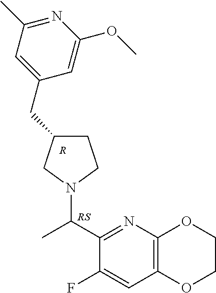 | |
| 91 | E60 | 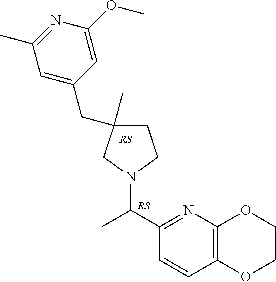 | |
| 92 | E60 | 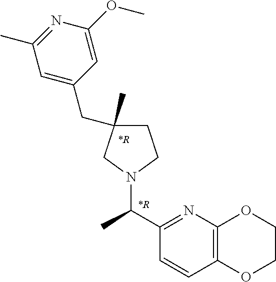 | |
| 93 | E60 | 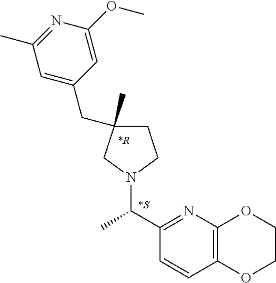 | |
| 94 | E60 | 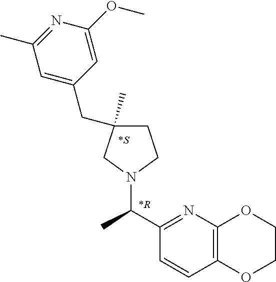 | |
| 95 | E60 | 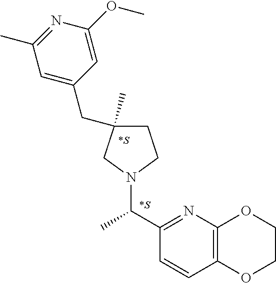 | |
| 96 | E61 | 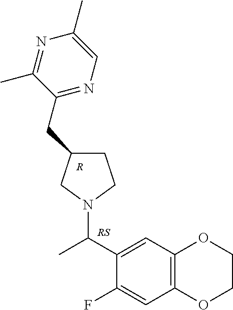 | |
| 97 | E61 | 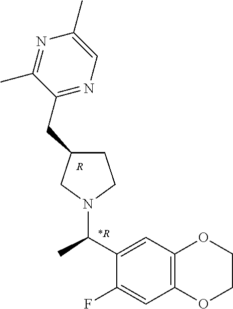 | |
| 98 | E61 | 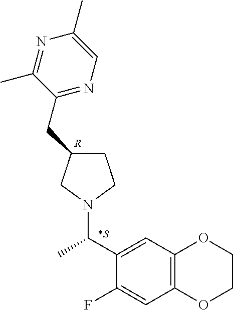 | |
| 100 | E51 | 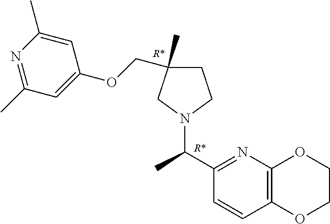 | |
| 101 | E51 | 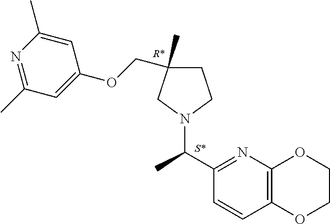 | |
| 102 | E51 | 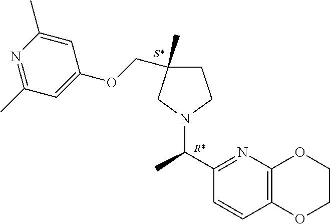 | |
| 103 | E51 | 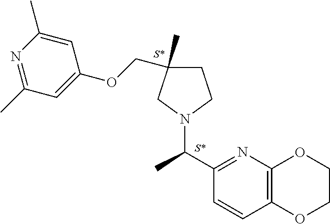 | •3HCl |
| 104 | E52 | 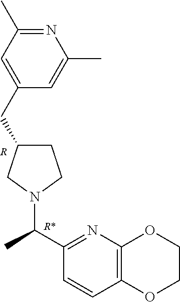 | •2HCl |
| 105 | E52 | 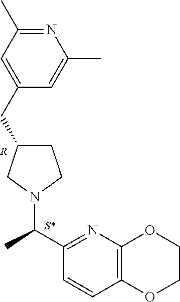 | •2HCl |
| 106 | E58 | 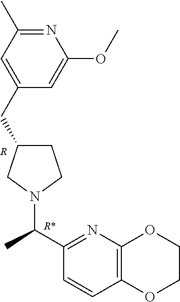 | |
| 107 | E58 | 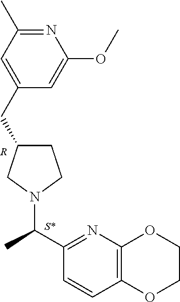 | |
| 115 | E68 | 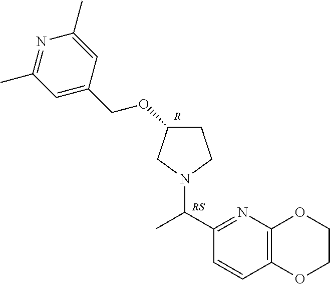 | |
| 116 | E69 | 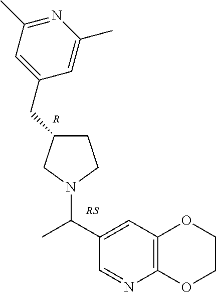 | |
| 117 | E70 | 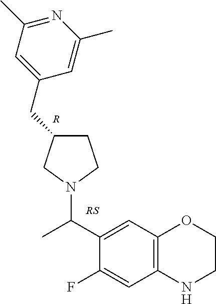 | |
| 118 | E71 | 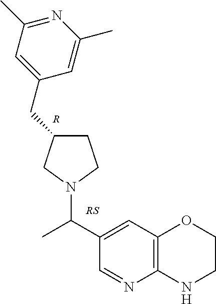 | |
| 119 | E72 | 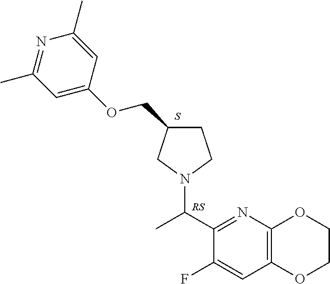 | |
| 120 | E73 | 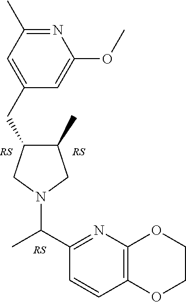 | |
| 121 | E73 | 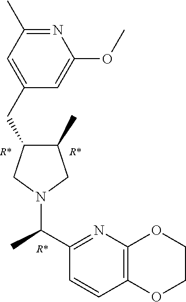 | |
| 122 | E73 | 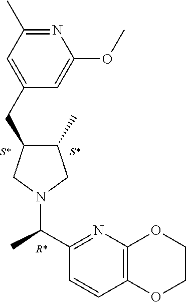 | |
| 123 | E73 | 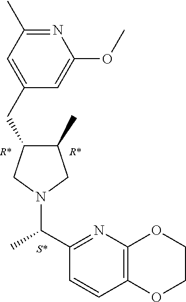 | |
| 124 | E73 | 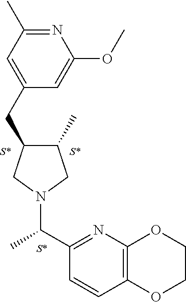 | |
| E74 | 125 | 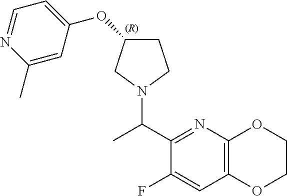 | 2HCl |
| E75 | 126 | 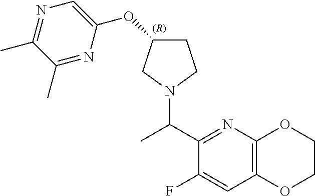 | 2HCl |
| E76 | 127 | 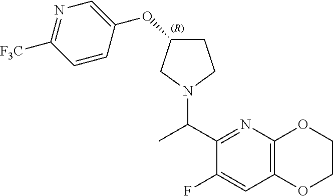 | HCl |
| E77 | 128 | 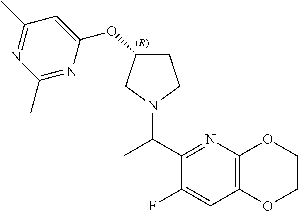 | HCl |
| E78 | 129 | 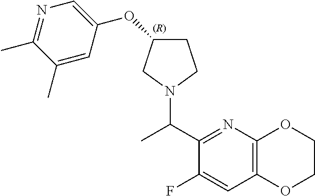 | HCl |

The values of salt stoichiometry or acid content in the compounds as provided herein, are those obtained experimentally. The content of hydrochloric acid reported herein was determined by ¹H NMR integration and/or elemental analysis.

### Analytical Part

### Melting Points

Values are peak values, and are obtained with experimental uncertainties that are commonly associated with this analytical method.

DSC823e (A): For a number of compounds, melting points were determined with a DSC823e (Mettler-Toledo) apparatus. Melting points were measured with a temperature gradient of 10° C./minute. Maximum temperature was 300° C. Values are peak values (A).

Mettler Toledo MP50 (B): For a number of compounds, melting points were determined in open capillary tubes on a Mettler FP 81HT/FP90 apparatus. Melting points were measured with a temperature gradient of 1, 3, 5 or 10° C./minute. Maximum temperature was 300° C. The melting point was read from a digital display.

### LCMS

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻ etc. . . . ). For molecules with multiple isotopic patterns (Br, Cl.), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, HSS” High Strength Silica, “CSH” charged surface hybrid, “UPLC” Ultra Performance Liquid Chromatography, “DAD” Diode Array Detector.

TABLE 2
LC-MS Methods (Flow expressed in mL/min; column temperature (T) in ° C.; Run time in min).

| Method code | Instrument | Column | Mobile phase | Gradient | $\frac{Flow}{Col T}$ | Run time |
|---|---|---|---|---|---|---|
| 1 | Waters: Acquity ® IClass UPLC ®- | Waters: BEH C18 (1.7 μm, 2.1 × 50 mm) | A: 95% CH₃COONH₄ 6.5 mM + 5% | From 95% A to 5% A in 4.6 min, held | $\frac{1}{50}$ | 5 |
| | DAD and | | CH₃CN, B: | for 0.4 min | | |
| | Xevo G2-S QTOF | | CH₃CN | | | |
| 2 | Agilent: HP1100-DAD, MSD G1956B | Agilent: Eclipse Plus C18 (3.5 μm, | A: 95% CH₃COONH₄ 6.5 mM + 5% | From 95% A to 0% A in 5.0 min, held | $\frac{1}{60}$ | 7 |
| | | 2.1 × 30 mm) | CH₃CN, B: | for 0.15 min, | | |
| | | | CH₃CN | back to 95% | | |
| | | | | A in 0.15 min, | | |
| | | | | held for 1.7 min | | |
| 3 | Waters: Acquity UPLC ®-DAD and Quattro | Waters: BEH C18 (1.7 μm, 2.1 × 100 mm) | A: 95% CH₃COONH₄ 7 mM/5% | 84.2% A for 0.49 min, to 10.5% A in 2.18 | $\frac{0.343}{40}$ | 6.2 |
| | Micro ™ | | CH₃CN, B: | min, held for | | |
| | | | CH₃CN | 1.94 min, back | | |
| | | | | to 84.2% A in | | |
| | | | | 0.73 min, held | | |
| | | | | for 0.73 min. | | |
| 4 | Waters: Acquity ® UPLC ®-DAD | Waters: BEH C18 (1.7 μm, 2.1 × 50 mm) | A: 95% CH₃COONH₄ 6.5 mM + 5% | From 95% A to 40% A in 1.2 min, to | $\frac{1}{50}$ | 2 |
| | and SQD | | CH₃CN, B: | 5% A in | | |
| | | | CH₃CN | 0.6 min, held | | |
| | | | | for 0.2 min | | |
| 5 | Agilent: 1100- DAD and MSD | YMC: Pack ODS-AQ (3 μm, | A: HCOOH 0.1% in water, B: CH₃CN | 95% A to 5% A in 4.8 min, held for | $\frac{2.6}{35}$ | 6 |
| | | 4.6 × 50 mm) | | 1 min, back to | | |
| | | | | 95% A in | | |
| | | | | 0.2 min. | | |
| 6 | Agilent 1260 Infinity DAD TOF-LC/MS | YMC-pack ODS-AQ C18 (50 × 4.6 | A: 0.1% HCOOH in H₂O | From 95% A to 5% A in 4.8 min, held | $\frac{2.6}{35}$ | 6.8 |
| | G6224A | mm, 3 μm) | B: CH₃CN | for 1.0 min, | | |
| | | | | to 95% A in | | |
| | | | | 0.2 min. | | |
| 7 | Waters: Acquity ® UPLC ®-DAD | Waters: BEH C18 (1.7 μm, 2.1 × 50 mm) | A: 95% CH₃COONH₄ 6.5 mM + 5% | From 95% A to 5% A in 2.0 min, held | $\frac{0.8}{50}$ | 2.5 |
| | and SQD | | CH₃CN, | for 0.5 min | | |
| | | | B: CH₃CN | | | |
| 8 | Waters: Acquity ® IClass UPLC ®- | Agilent: RRHD (1.8 μm, | A: 95% CH₃COONH₄ 6.5 mM + 5% | From 95% A to 5% A in 2.0 min, held | $\frac{0.8}{50}$ | 2.5 |
| | DAD and SQD | 2.1 × 50 mm) | CH₃CN, B: | for 0.5 min | | |
| | | | CH₃CN | | | |
| 9 | Waters: Acquity ® UPLC ®-DAD | Waters: BEH C18 (1.7 μm, 2.1 × 50 mm) | A: 95% CH₃COONH₄ 6.5 mM + 5% | From 95% A to 5% A in 4.5 min, held | $\frac{0.8}{50}$ | S60 53S 600 6 |
| | and SQD | | CH₃CN, B: | for 0.5 min | | |
| | | | CH₃CN | | | |

TABLE 3
Analytical data - melting point (M.p.) and LCMS:
[M + H]⁺ means the protonated mass of the
free base of the compound, [M − H]⁻ means
the deprotonated mass of the free base of the compound
or the type of adduct specified [M + CH₃COO]⁻).
Rₜ means retention time (in min). For some compounds, exact
mass was determined.

| Co. | | | | LCMS |
|---|---|---|---|---|
| No. | M.p. (° C.) | [M + H]⁺ | Rₜ | Method |
| 70 | n.d. | 356 | 1.64 | 1 |
| 71 | n.d. | 356 | 1.23 | 1 |
| 72 | n.d. | 356 | 1.26 | 1 |
| 73 | n.d. | 356 | 1.30/1.34 | 1 |
| 74 | n.d. | 356 | 2.16 | 3 |
| 75 | n.d. | 356 | 2.15 | 3 |
| 76 | n.d. | 372 | 1.70 | 1 |
| 77 | n.d. | 384 | 2.20 | 1 |
| 78 | n.d. | 354 | 1.05 | 1 |
| 79 | n.d. | 372 | 1.31 | 1 |
| 80 | n.d. | 353 | 1.03/1.06 | 1 |
| 81 | n.d. | 386 | 1.05 | 7 |
| 82 | n.d. | 386 | 1.08 | 7 |
| 83 | n.d. | 372 | 2.48 | 3 |
| 84 | n.d. | 372 | 2.52 | 3 |
| 85 | n.d. | 372 | 2.50 | 3 |
| 86 | n.d. | 368 | 2.38 | 3 |
| 87 | n.d. | 368 | 2.38 | 3 |
| 88 | n.d. | 368 | 2.38 | 3 |
| 89 | n.d. | 370 | 2.26 | 3 |
| 90 | n.d. | 388 | 1.68 | 1 |
| 91 | n.d. | 384 | 2.55 | 3 |
| 92 | n.d. | 384 | 2.56 | 3 |
| 93 | n.d. | 384 | 2.54 | 3 |
| 94 | n.d. | 384 | 2.54 | 3 |
| 95 | n.d. | 384 | 2.54 | 3 |
| 96 | n.d. | 372 | 2.39/2.43 | 3 |
| 97 | n.d. | 372 | 2.38 | 3 |
| 98 | n.d. | 372 | 2.42 | 3 |
| 100 | n.d. | 384 | 1.33 | 1 |
| 101 | n.d. | 384 | 1.34 | 1 |
| 102 | n.d. | 384 | 2.22 | 3 |
| 103 | n.d. | 384 | 1.34 | 1 |
| 104 | n.d. | 354 | 1.93 | 3 |
| 105 | n.d. | 354 | 1.93 | 3 |
| 106 | n.d. | 370 | 2.27 | 3 |
| 107 | n.d. | 370 | 2.26 | 3 |
| 115 | n.d. | 370 | 1.18 | 1 |
| 116 | n.d. | 354 | 1.17/1.21 | 1 |
| 117 | n.d. | 370 | 1.25/1.28 | 1 |
| 118 | n.d. | 353 | 0.97/1.00 | 1 |
| 119 | n.d. | 388 | 1.38 | 1 |
| 120 | n.d. | 384 | 1.53 | 1 |
| 121 | n.d. | 384 | 2.44 | 3 |
| 122 | n.d. | 384 | 2.45 | 3 |
| 123 | n.d. | 384 | 2.43 | 3 |
| 124 | n.d. | 384 | 2.45 | 3 |
| 45 | n.d. | 371.2 | 1.79 | 1 |
| 1 | 297.50° C. (A) | 366.1 | 2.68 | 2 |
| 2 | n.d. | 352 | 2.46 | 2 |
| 3 | n.d. | 340.2 | 1.16 | 1 |
| 4 | n.d. | 424.19 | 2.12 | 1 |
| 4 | n.d. | 424.2 | 2.11 | 1 |
| 5 | n.d. | 408.19 | 1.67 | 1 |
| 6 | n.d. | 391.2000/391.1997 | 2.25/2.32 | 1 |
| 7 | n.d. | 425.1866/425.1867 | 2.62/2.70 | 1 |
| 8 | n.d. | 353 | 1.95/1.98 | 1 |
| 9 | n.d. | 370.2 | 1.33 | 1 |
| 9 | n.d. | 370 | 0.93 | 7 |
| 10 | n.d. | 387 | 2.33 | 1 |
| 11 | n.d. | 387 | 2.39 | 1 |
| 12 | n.d. | 354.2 | 1.09 | 1 |
| 13 | n.d. | 337.2 | 1.56 | 1 |
| 14 | n.d. | 337.2 | 1.6 | 1 |
| 15 | n.d. | 351.2 | 0.86 | 1 |
| 16 | n.d. | 388.2 | 1.17 | 8 |
| 17 | n.d. | 358.3 | 1 | 7 |
| 18 | n.d. | 358.3 | 0.99 | 7 |
| 18 | n.d. | 358.2 | 2.39 | 3 |
| | | 418.4 M + | | |
| | | (CH3COO)— | | |
| 19 | n.d. | 358.3 | 1 | 7 |
| 19 free | n.d. | 358.2 | 2.39 | 3 |
| base | | 418.3 M + | | |
| | | (CH3COO)— | | |
| 20 | n.d. | 369.2 | 1.33, 1.36 | 1 |
| 21 | n.d. | 362 | 2.12 | 3 |
| 21 | n.d. | 362.2 | 1.25 | 1 |
| 22 | n.d. | 362 | 2.12 | 3 |
| 23 | n.d. | 362 | 2.12 | 3 |
| 24 | n.d. | 355 | 1.8 | 3 |
| 24 | n.d. | 355.2 | 0.97, 0.99 | 1 |
| 25 | n.d. | 355 | 1.79 | 3 |
| 26 | n.d. | 355 | 1.78 | 3 |
| 27 | n.d. | 379 | 2.87, 2.92 | 3 |
| 27 | n.d. | 379.2 | 2.04, 2.08 | 1 |
| 28 | n.d. | 379 | 2.87 | 3 |
| | | 439.8 [M + | | |
| | | CH3COO]— | | |
| 29 | n.d. | 379 | 2.92 | 3 |
| | | 439.3 [M + | | |
| | | CH3COO]— | | |
| 30 | n.d. | 371.2 | 1.77/1.84 | 1 |
| 32 | n.d. | 371.2 | 1.84 | 1 |
| 33 | n.d. | 395.2 | 1.44-1.48 | 1 |
| 34 | n.d. | 395.2 | 1.44 | 1 |
| 35 | n.d. | 354.2 | 1.23 | 1 |
| 36 | n.d. | 354.2 | 1.17 | 1 |
| 37 | n.d. | 355.2 | 0.81 | 1 |
| 39 | n.d. | 374.2 | 1.57/1.59 | 1 |
| 38 | n.d. | 374.2 | 1.55/1.58 | 1 |
| 40 | n.d. | 374.2 | 1.57 | 1 |
| 31 | n.d. | 371.2 | 1.78 | 1 |
| 41 | n.d. | 374.2 | 1.56 | 1 |
| 42 | n.d. | 387.2 | 2.29 | 1 |
| 43 | n.d. | 387.2 | 2.23 | 1 |
| 44 | n.d. | 371.2 | 1.82 | 1 |
| 46 | n.d. | 370.1 | 2.01 | 3 |
| 46 | n.d. | 370.2 | 1.1 | 1 |
| 47 | n.d. | 370.2 | 1.09 | 1 |
| 48 | n.d. | 386.2 | 1.46 | 1 |
| 49 | n.d. | 370.2 | 1.19 | 1 |
| 50 | n.d. | 388.2 | 1.39 | 1 |
| 51 | n.d. | 372.2 | 1.32 | 1 |
| 51 free | n.d. | 372.2 | 1.32 | 1 |
| base | | | | |
| 51 free | n.d. | 372.1 | 2.17 | 3 |
| base | | 432.3 [M + | | |
| | | CH3COO]— | | |
| 52 free | n.d. | 372.2 | 1.32 | 1 |
| base | | | | |
| 52 | n.d. | 372.2 | 1.31 | 1 |
| 52 free | n.d. | 372.1 | 2.18 | 3 |
| base | | 431.6 [M + | | |
| | | CH3COO]— | | |
| 53 | n.d. | 372.21 | 1.66/1.68 | 9 |
| 54 | n.d. | 354.2 | 1.37 | 1 |
| 55 | n.d. | 354.16 | 1.42 | 1 |
| 56 | n.d. | 342.2 | 1.3 | 1 |
| 57 | n.d. | 360.2 | 1.6 | 1 |
| 58 | n.d. | 368.2 | 1.2825 | 1 |
| 59 | n.d. | 368.2 | 1.27 | 1 |
| 60 | n.d. | 368.2 | 1.24 | 1 |
| 61 | n.d. | 368.2 | 1.21 | 1 |
| 62 | n.d. | 343.2 | 1.03/1.05 | 1 |
| 63 | n.d. | 372.2 | 1.67-1.70 | 1 |
| 64 | n.d. | 372 | 1.69 | 1 |
| 125 | n.d. | 360.2 | 1.51 and 1.52 | 1 |
| 125 free | n.d. | 360.2 | 1.53 and 1.54 | 1 |
| base | | | | |
| 126 | n.d. | 375.2 | 1.83 | 1 |
| 127 | n.d. | 414.1 | 2.19 and 2.20 | 1 |
| 128 | n.d. | 375.2 | 1.57 | 1 |
| 128 free | n.d. | 375.2 | 1.64 | 1 |
| base | | | | |
| 129 | n.d. | 374.2 | 1.75 | 1 |
| 129 free | n.d. | 374.2 | 1.74 | 1 |
| base | | | | |

### Optical Rotations

Optical rotations were measured on a Perkin-Elmer 341 polarimeter with a sodium lamp and reported as follows: [α]° (λ, c g/100 ml, solvent, T ° C.).

[α]_{λ}^{T}=(100α)/(l×c): where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation, the concentration and solvent are always provided in parentheses after the rotation. The rotation is reported using degrees and no units of concentration are given (it is assumed to be g/100 mL).

TABLE 4
Optical Rotation data.

| Co. | | Wavelength | Concentration | | Temp. |
|---|---|---|---|---|---|
| No. | α_{D} (°) | (nm) | w/v % | Solvent | (° C.) |
| 2 | +0.8° | 589 | 0.54 | MeOH | 20 |

### SFCMS-Methods

### General Procedure for SFC-MS Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 5
Analytical SFC-MS Methods (Flow expressed in mL/min; column
temperature (T) in ° C.; run time in minutes; backpressure (BPR) in bars.

| Method code | Column | Mobile phase | Gradient | $\frac{Flow}{Col T}$ | $\frac{Run time}{BPR}$ |
|---|---|---|---|---|---|
| 1 | Daicel Chiralpak ® AD-3 column (3 μm, 100 × 4.6 mm) | A: CO₂ B: MeOH (+0.3% iPrNH₂) | 20% B hold 3 min, | $\frac{3.5}{35}$ | $\frac{3}{103}$ |
| 2 | Daicel Chiralpak ® AD-3 column (3 μm, 100 × 4.6 mm) | A: CO₂ B: MeOH (+0.3% iPrNH₂) | 20% B hold 4 min, | $\frac{3.5}{35}$ | $\frac{4}{103}$ |
| 3 | Daicel Chiralpak ® AD-3 column (3 μm, 100 × 4.6 mm) | A: CO₂ B: EtOH (+0.3% iPrNH₂) | 10% B hold 6 min, | $\frac{3.5}{35}$ | $\frac{6}{103}$ |
| 4 | Daicel Chiralpak ® AD-3 column (3 μm, 100 × 4.6 mm) | A: CO₂ B: EtOH (+0.3% iPrNH₂) | 15% B hold 3 min, | $\frac{3.5}{35}$ | $\frac{3}{103}$ |
| 5 | Daicel Chiralpak ® AD-3 column (3 μm, 100 × 4.6 mm) | A: CO₂ B: EtOH (+0.3% iPrNH₂) | 20% B hold 3 min, | $\frac{3.5}{35}$ | $\frac{3}{103}$ |
| 6 | Daicel Chiralpak ® AD-3 column (3 μm, 100 × 4.6 mm) | A: CO₂ B: iPrOH (+0.3% iPrNH₂) | 10% B hold 6 min, | $\frac{3.5}{35}$ | $\frac{6}{103}$ |
| 7 | Daicel Chiralpak ® AD-3 column (3 μm, 100 × 4.6 mm) | A: CO₂ B: iPrOH (+0.3% iPrNH₂) | 15% B hold 3 min, | $\frac{3.5}{35}$ | $\frac{3}{103}$ |
| 8 | Daicel Chiralcel ® OJ-3 column (3 μm, 100 × 4.6 mm) | A: CO₂ B: MeOH (+0.3% iPrNH₂) | 10% B hold 3 min, | $\frac{3.5}{35}$ | $\frac{3}{103}$ |
| 9 | Daicel Chiralcel ® OJ-3 column (3 μm, 100 × 4.6 mm) | A: CO₂ B: MeOH (+0.3% iPrNH₂) | 20% B hold 3 min, | $\frac{3.5}{35}$ | $\frac{3}{103}$ |
| 10 | Daicel Chiralcel ® OJ-3 column (3 μm, 100 × 4.6 mm) | A: CO₂ B: MeOH (+0.3% iPrNH₂) | 25% B hold 3 min, | $\frac{3.5}{35}$ | $\frac{3}{103}$ |
| 11 | Daicel Chiralcel ® OJ-3 column (3 μm, 100 × 4.6 mm) | A: CO₂ B: EtOH (+0.3% iPrNH₂) | 10% B hold 6 min, | $\frac{3.5}{35}$ | $\frac{6}{103}$ |
| 12 | Daicel Chiralcel ® OJ-3 column (3 μm, 100 × 4.6 mm) | A: CO₂ B: iPrOH (+0.3% iPrNH₂) | 20% B hold 3 min, | $\frac{3.5}{35}$ | $\frac{3}{103}$ |
| 13 | Daicel Chiralcel ® OJ-3 column (3 μm, 100 × 4.6 mm) | A: CO₂ B: iPrOH (+0.3% iPrNH₂) | 25% B hold 3 min, | $\frac{3.5}{35}$ | $\frac{3}{103}$ |
| 14 | Daicel Chiralpak ® IC-3 column (3 μm, 100 × 4.6 mm) | A: CO₂ B: iPrOH (+0.3% iPrNH₂) | 40% B hold 3 min, | $\frac{3.5}{35}$ | $\frac{3}{103}$ |
| 15 | Daicel Chiralpak ® IC-3 column (3 μm, 100 × 4.6 mm) | A: CO₂ B: iPrOH (+0.3% iPrNH₂) | 50% B hold 6 min, | $\frac{3.5}{35}$ | $\frac{6}{103}$ |
| 16 | Daicel Chiralpak ® OD-3 column (3 μm, 100 × 4.6 mm) | A: CO₂ B: iPrOH (+0.3% iPrNH₂) | 20% B hold 3 min, | $\frac{3.5}{35}$ | $\frac{3}{103}$ |
| 17 | Phenomenex Lux cellulose 4 column (3 μm, 100 × 4.6 mm) | A: CO₂ B: EtOH (+0.3% iPrNH₂) | 30% B hold 3 min, | $\frac{3.5}{35}$ | $\frac{3}{103}$ |
| 18 | Phenomenex Lux cellulose 4 column (3 μm, 100 × 4.6 mm) | A: CO₂ B: iPrOH (+0.3% iPrNH₂) | 30% B hold 6 min, | $\frac{3.5}{35}$ | $\frac{6}{103}$ |
| 19 | Daicel Chiralpak ® IC-3 column (3 μm, 100 × 4.6 mm) | A: CO₂ B: iPrOH (+0.3% iPrNH₂) | 30% B hold 3 min, | $\frac{3.5}{35}$ | $\frac{3}{103}$ |
| 20 | Daicel Chiralpak ® AD-3 column (3 μm, 100 × 4.6 mm) | A: CO₂ B: EtOH (+0.3% iPrNH₂) | 10% B hold 3 min, | $\frac{3.5}{35}$ | $\frac{3}{103}$ |
| 21 | Daicel Chiralcel ® OJ-3 column (3 μm, 100 × 4.6 mm) | A: CO₂ B: MeOH (+0.3% iPrNH₂) | 30% B hold 3 min, | $\frac{3.5}{35}$ | $\frac{3}{103}$ |
| 22 | Daicel Chiralcel ® OJ-3 column (3 μm, 100 × 4.6 mm) | A: CO₂ B: MeOH (+0.3% iPrNH₂) | 15% B hold 3 min, | $\frac{3.5}{35}$ | $\frac{3}{103}$ |
| 23 | Daicel Chiralpak ® IG-3 column (3 μm, 100 × 4.6 mm) | A: CO₂ B: MeOH (+0.3% iPrNH₂) | 30% B hold 3 min, | $\frac{3.5}{35}$ | $\frac{3}{103}$ |

TABLE 6
Analytical SFC data - Rₜ means retention time (in
minutes), [M + H]⁺ means the protonated
mass of the compound, method refers to the method
used for (SFC)MS analysis of enantiomerically pure compounds.

| Co. | | | UV | | Isomer Elution |
|---|---|---|---|---|---|
| Nr. | Rₜ | [M + H]⁺ | Area % | Method | Order |
| 71 | 1.05 | 356 | 100 | 9 | A |
| 72 | 1.50 | 356 | 100 | 9 | B |
| 74 | 0.79 | 356 | 100 | 1 | A |
| 75 | 0.94 | 356 | 100 | 1 | B |
| 84 | 1.02 | 372 | 100 | 12 | A |
| 85 | 1.25 | 372 | 99.3 | 12 | B |
| 86 | 0.95 | 368 | 100 | 5 | A |
| 87 | 2.00 | 368 | 99.1 | 5 | B |
| 92 | 2.63 | 384 | 90.6 | 6 | A |
| 93 | 3.15 | 384 | 98.6 | 6 | B |
| 94 | 1.38 | 384 | 98.4 | 8 | A |
| 95 | 1.70 | 384 | 97 | 8 | B |
| 97 | 1.11 | 372 | 100 | 4 | A |
| 98 | 1.61 | 372 | 100 | 4 | B |
| 100 | 1.40 | 384 | 100 | 13 | A |
| 101 | 1.83 | 384 | 100 | 13 | B |
| 102 | 1.46 | 384 | 100 | 17 | A |
| 103 | 1.60 | 384 | 98.9 | 17 | B |
| 104 | 1.28 | 354 | 100 | 4 | A |
| 105 | 1.42 | 354 | 95.2 | 4 | B |
| 106 | 2.38 | 370 | 100 | 3 | A |
| 107 | 2.69 | 370 | 94 | 3 | B |
| 121 | 0.97 | 384 | 100 | 12 | A |
| 122 | 1.64 | 384 | 100 | 19 | A |
| 123 | 1.94 | 384 | 100 | 19 | B |
| 124 | 1.55 | 384 | 100 | 12 | B |
| 27 | 0.98, 1.36 | 379 | 46.43, 53.57 | 20 | 20 |
| 24 | 0.99, 1.53 | 355 | 49.79, 50.21 | 20 | |
| 21 | 0.92, 1.60 | 362 | 52.01, 47.99 | 13 | |
| 28 | 0.98 | 379 | 100.00 | 20 | A |
| 29 | 1.36 | 379 | 99.06 | 20 | B |
| 25 | 0.99 | 355 | 100.00 | 21 | A |
| 26 | 1.53 | 355 | 100.00 | 21 | B |
| 23 | 0.93 | 362 | 100.00 | 13 | A |
| 22 | 1.63 | 362 | 99.89 | 13 | B |
| 46 | 1.26, 1.89 | 370 | 50.24, 49.76 | 21 | |
| 52 free | 0.87 | 372 | 100.00 | 22 | A |
| base | | | | | |
| 51 free | 1.13 | 372 | 99.14 | 22 | B |
| base | | | | | |
| 18 free | 1.23 | 358 | 100.00 | 23 | A |
| base | | | | | |
| 19 free | 1.60 | 358 | 100.00 | 23 | B |
| base | | | | | |

### NMR

For a number of compounds, ¹H NMR spectra were recorded on a Bruker Avance III with a 300 MHz Ultrashield magnet, on a Bruker DPX-400 spectrometer operating at 400 MHz, on a Bruker Avance I operating at 500 MHz, on a Bruker DPX-360 operating at 360 MHz, or on a Bruker Avance 600 spectrometer operating at 600 MHz, using CHLOROFORM-d (deuterated chloroform, CDCl₃) or DMSO-d₆ (deuterated DMSO, dimethyl-d₆ sulfoxide) as solvent. Chemical shifts (δ) are reported in parts per million (ppm) relative to tetramethylsilane (TMS), which was used as internal standard.

TABLE 6
¹H NMR results

| Co. | |
|---|---|
| No. | ¹H NMR result |
| 125 | ¹H NMR (400 MHz, DMSO-d6) δ ppm 1.57 (dd, J = 6.36, 4.74 Hz, 3 H) 2.04- |
| | 2.42 (m, 2 H) 2.68 (s, 4 H) 3.17-3.37 (m, 1 H) 3.60-4.23 (m, 2 H) 4.23- |
| | 4.62 (m, 4 H) 4.86 (br dd, J = 12.25, 6.01 Hz, 1 H) 5.30-5.66 (m, 1 H) 7.12- |
| | 7.87 (m, 3 H) 8.64 (d, J = 6.94 Hz, 2 H) 11.07-11.96 (m, 1 H) 15.08-16.14 |
| | (m, 1 H) |
| 128 | ¹H NMR (400 MHz, DMSO-d6) δ ppm 1.48-1.64 (m, 3 H) 2.05-2.32 (m, 1 |
| | H) 2.53 (br d, J = 3.01 Hz, 3 H) 2.60-2.72 (m, 3 H) 3.18-3.67 (m, 4 H) 4.26- |
| | 4.52 (m, 5 H) 4.72-4.90 (m, 1 H) 5.56-5.74 (m, 1 H) 6.79-7.05 (m, 1 H) |
| | 7.43-7.63 (m, 1 H) 11.07-11.66 (m, 1 H) |
| 62 | ¹H NMR (500 MHz, CHLOROFORM-d) δ ppm 1.37-1.43 (m, 3 H) 1.91- |
| | 2.02 (m, 1 H) 2.26-2.36 (m, 1 H) 2.42-2.99 (m, 7 H) 3.37-3.46 (m, 1 H) |
| | 4.22-4.28 (m, 2 H) 4.41-4.46 (m, 2 H) 4.76-4.88 (m, 1 H) 6.93-7.02 (m, |
| | 1 H) 7.12-7.20 (m, 1 H) 8.13-8.33 (m, 2 H) |
| 57 | ¹H NMR (400 MHz, CHLOROFORM-d) δ ppm 1.47 (dd, J = 6.70, 3.70 Hz, 3 |
| | H) 1.84-2.01 (m, 1 H) 2.17-2.34 (m, 1 H) 2.47 (d, J = 2.08 Hz, 3 H) 2.50- |
| | 2.78 (m, 2 H) 2.83-2.94 (m, 1 H) 2.98-3.25 (m, 1 H) 3.92-4.05 (m, 1 H) |
| | 4.12-4.32 (m, 2 H) 4.35-4.47 (m, 2 H) 4.69-4.83 (m, 1H) 6.96 (d, J = 9.02 |
| | Hz, 1 H) 7.00-7.09 (m, 2 H) 8.10 (dt, J = 12.72, 1.85 Hz, 1 H) |
| 40 | ¹H NMR (400 MHz, DMSO-d6) δ ppm 1.56 (br d, J = 6.47 Hz, 3 H) 2.05- |
| | 2.44 (m, 2 H) 2.65 (br s, 6 H) 3.45-3.62 (m, 2 H) 3.65-4.08 (m, 2 H) 4.28- |
| | 4.65 (m, 4 H) 4.86 (br s, 1 H) 5.23-5.71 (m, 1 H) 7.12-7.41 (m, 2 H) 7.58 |
| | (br d, J = 9.48 Hz, 1 H) 11.00-1.88 (m, 1 H) 14.90-15.70 (m, 1 H) |
| 52 | ¹H NMR (500 MHz, DMSO-d6) δ ppm 1.54 (d, J = 6.65 Hz, 3 H) 1.58-1.64 |
| | 8 m, 1 H) 1.65-1.78 (m, 1 H) 1.94-2.08 (m, 1 H) 2.64-2.85 (m, 1 H) 2.86- |
| | 3.07 (m, 2 H) 3.23-3.37 (m, 2 H) 3.54-3.65 (m, 1 H) 4.29-4.39 (m, 2 H) |
| | 4.41-4.54 (m, 2 H) 4.74 (dq, J = 14.21, 6.92, 6.92, 6.92, 6.92, Hz, 1H) 7.54 (br |
| | d, J = 1.44 Hz, 1 H) 7.53-7.66 (m, 2 H) 10.66-10.99 (m, 1 H) 15.47-15.99 |
| | (m, 1 H) |
| 50 | ¹H NMR (500 MHz, CHLOROFORM-d) δ ppm 1.36-1.49 (m, 3 H) 1.49- |
| | 1.56 (m, 1 H) 1.94-2.08 (m, 1 H) 2.28-2.41 (m, 3 H) 2.45 (s, 6 H) 2.51 (br |
| | s, 1 H) 2.56-2.68 (m, 1 H) 2.68-2.84 (m, 2 H) 2.94 (br d, J = 6.94 Hz, 3 H) |
| | 4.18-4.29 (m, 2 H) 4.40 (br dd, J = 3.47, 2.02 Hz, 2 H) 6.46 (s, 2 H) 6.94 (br |
| | d, J = 9.25 Hz, 1 H) 6.95 (br d, J = 9.25 Hz, 1 H) |

### Pharmacological Examples

### 1) OGA—Biochemical Assay

The assay is based on the inhibition of the hydrolysis of fluorescein mono-β-D-N-Acetyl-Glucosamine (FM-GlcNAc) (Mariappa et al. 2015, Biochem J 470:255) by the recombinant human Meningioma Expressed Antigen 5 (MGEA5), also referred to as O-GlcNAcase (OGA). The hydrolysis FM-GlcNAc (Marker Gene technologies, cat #M1485) results in the formation of β-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OriGene (cat #TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄ 2 H₂O (Sigma, #C0759) were dissolved in 1 L water to obtain a 200 mM solution. 19.2 g citric acid (Merck, #1.06580) was dissolved in 1 L water to obtain a 100 mM solution. pH of the sodium phosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at a 2 nM concentration and FM-GlcNAc at a 100 uM final concentration. Dilutions were prepared in assay buffer.

50 nl of a compound dissolved in DMSO was dispensed on Black Proxiplate™ 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000 rpm and incubate at room temperature for 6 h. To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

### 2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting O-GlcNAcylated residues as previously described (Dorfmueller et al. 2010 Chemistry & biology, 17:1250). Inhibition of OGA will result in an increase of O-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, #D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100 μl of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10 fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, #D8537). From next step onwards unless other stated assay volume was always 50 μl and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, #A16046), pH 7.5 (ICC buffer) before being permeabilized in same buffer for 10 minutes. Samples are subsequently blocked in ICC containing 5% goat serum (Sigma, #G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, #A-21042) and nuclei stained with Hoechst 33342 at a final concentration of 1 μg/ml in ICC (Lifetechnologies, #H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 7
Results in the biochemical and cellular assays.

| | Enzymatic | Enzymatic | Cellular | Cellular |
|---|---|---|---|---|
| Co. | hOGA; | Eₘₐₓ | hOGA; | Eₘₐₓ |
| No. | pIC₅₀ | (%) | pEC₅₀ | (%) |
| 1 | 5.7 | 85 | | |
| 2 | 5.21 | 61 | | |
| 3 | 6.3 | 99 | | |
| 4 | 7.0 | 103 | | |
| 5 | 6.9 | 100 | 6 | 45 |
| 6 | 7.1 | 103 | <6 | 47 |
| 7 | 7.7 | 102 | 6.4 | 79 |
| 8 | 6.9 | 101 | 6.1 | 58 |
| 9 | 6.9 | 100 | 6.1 | 51 |
| 10 | 5.8 | 88 | <6 | −5 |
| 11 | 7.6 | 102 | 6.7 | 83 |
| 12 | 6.8 | 101 | | |
| 13 | 6.0 | 91 | | |
| 14 | 7.3 | 103 | 6.9 | 74 |
| 15 | 6.3 | 95 | <6 | 45 |
| 16 | 7.7 | 102 | 6.9 | 89 |
| 17 | 8.6 | 101 | 7.7 | 77 |
| 18 | 6.8 | 98 | 6.0 | 42 |
| 19 | 8.8 | 97 | 7.73 | 94 |
| 20 | 7.1 | 101 | 6.3 | 61 |
| 21 | 6.2 | 94 | | |
| 22 | <5 | 32 | | |
| 23 | 6.4 | 97 | | |
| 24 | 6.6 | 101 | | |
| 25 | 6.8 | 100 | | |
| 26 | <5 | 35 | | |
| 27 | 6.9 | 102 | | |
| 28 | 5.3 | 67 | | |
| 29 | 7.3 | 102 | <6 | 35 |
| 30 | 7.7 | 102 | | |
| 31 | 6.2 | 96 | | |
| 32 | 7.8 | 101 | 6.9 | 72 |
| 33 | 6.9 | 102 | | |
| 34 | 5.1 | 58 | | |
| 35 | 5.1 | 54 | <6 | −6 |
| 36 | 6.7 | 99 | <6 | 15 |
| 37 | 6.6 | 96 | <6 | 14 |
| 38 | 7.7 | 98 | 6.5 | 69 |
| 39 | 8.0 | 100 | 6.6 | 74 |
| 40 | 8.1 | 100 | 6.8 | 84 |
| 41 | 5.8 | 87 | <6 | −13 |
| 42 | 6.0 | 93 | | |
| 43 | 7.8 | 102 | 6.4 | 68 |
| 44 | 6.6 | 99 | | |
| 45 | 8.3 | 101 | 7.0 | 72 |
| 46 | 6.6 | 97 | | |
| 47 | 6.2 | 97 | <6 | 9 |
| 48 | 6.0 | 91 | <6 | 1 |
| 49 | 5.3 | 70 | <6 | −5 |
| 50 | 7.3 | 100 | 6.2 | 58 |
| 51 | 6.4 | 96 | <6 | 8 |
| 52 | 8.2 | 101 | 7.2 | 80 |
| 53 | 7.2 | 93 | <6 | 43 |
| 54 | <5 | 19 | <6 | 2 |
| 55 | 6.7 | 94 | <6 | 24 |
| 56 | 5.6 | 83 | <6 | 8 |
| 57 | 6.5 | 96 | <6 | 33 |
| 58 | 6.5 | 100 | <6 | 17 |
| 59 | 6.4 | 97 | <6 | 34 |
| 60 | 7.1 | 99 | <6 | 44 |
| 61 | 6.9 | 97 | 6.1 | 56 |
| 62 | 5.6 | 86 | <6 | 1 |
| 63 | 6.0 | 95 | | |
| 64 | 6.4 | 99 | | |
| 70 | 5.9 | 94 | | |
| 71 | 6.1 | 99 | <6 | 9 |
| 72 | <5 | 21 | <6 | −8 |
| 73 | 7.1 | 99 | <6 | 33 |
| 74 | 5.1 | 50 | <6 | −9 |
| 75 | 7.4 | 99 | <6 | 34 |
| 76 | 5.8 | 84 | | |
| 77 | 6.1 | 94 | | |
| 78 | 7.2 | 101 | | |
| 79 | 8.1 | 98 | 7.0 | 85 |
| 80 | 7.4 | 101 | 6.4 | 71 |
| 81 | 6.1 | 92 | | |
| 82 | 8.0 | 101 | 6.6 | 81 |
| 83 | 8.3 | 102 | 7.5 | 89 |
| 84 | 8.5 | 102 | 7.8 | 100 |
| 85 | 6.7 | 99 | | |
| 86 | 6.8 | 101 | 6.13 | 50 |
| 87 | 6.9 | 99 | | |
| 88 | <5 | 38 | | |
| 89 | 7.0 | 100 | | |
| 90 | 7.9 | 101 | 6.9 | 90 |
| 91 | 6.8 | 100 | | |
| 92 | 5.2 | 63 | | |
| 93 | 5.1 | 55 | | |
| 94 | 6.0 | 93 | | |
| 95 | 7.1 | 100 | <6 | 39 |
| 96 | 7.2 | 101 | | |
| 97 | <5 | 37 | | |
| 98 | 7.5 | 103 | | |
| 100 | 5.7 | 87 | <6 | −5 |
| 101 | <5 | 13 | <6 | −4 |
| 102 | 6.4 | 93 | <6 | 7 |
| 103 | <5 | 29 | <6 | −5 |
| 104 | 7.2 | 100 | 6.2 | 51 |
| 105 | 6.1 | 91 | <6 | 6 |
| 106 | 7.1 | 100 | 6.0 | 47 |
| 107 | 5.9 | 90 | <6 | −6 |
| 115 | 6.2 | 94 | <6 | 9 |
| 116 | 6.8 | 97 | <6 | 20 |
| 117 | 7.8 | 99 | 6.5 | 60 |
| 118 | 7.5 | 100 | 6.2 | 49 |
| 119 | 7.0 | 100 | 6.2 | 59 |
| 120 | 6.8 | 100 | <6 | 36 |
| 121 | 7.3 | 99 | <6 | 47 |
| 122 | 5.0 | 49 | <6 | −7 |
| 123 | 6.9 | 100 | ~6 | 47 |
| 124 | <5 | 40 | <6 | −3 |
| 125 | 6.8 | 89 | 6.3 | 48 |
| 126 | 7.0 | 94 | 6.3 | 73 |
| 127 | 6.7 | 96 | <6 | 26 |
| 128 | 7.3 | 95 | 6.36 | 59 |
| 129 | 6.7 | 91 | 6.0 | 45 |

1. A compound of Formula (I) 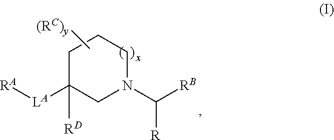
or a tautomer or a stereoisomeric form thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents;
—C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —CH₂—, —O—, —OCH₂—, —CH₂O—, —NH—, —N(CH₃)—, —NHCH₂— and —CH₂NH—;
x represents 0;
R is H or CH₃; and
R^{B} is a bicyclic radical of formula (b-1), (b-2) or (b-3) 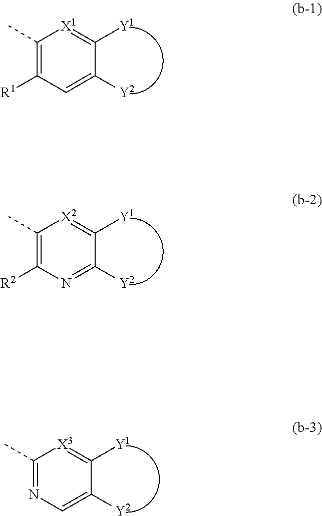
wherein
R¹ and R² are each selected from the group consisting of hydrogen, fluoro and methyl;
X¹, X² and X³ each represent CH, CF or N;
—Y¹—Y²— forms a bivalent radical selected from the group consisting of

| | | | |
|---|---|---|---|
| —O(CH₂)ₘO— | (c-1); | | |
| —O(CH₂)ₙ— | (c-2); | —(CH₂)ₙO— | (c-3); |
| —O(CH₂)ₚNR³— | (c-4); | —NR³(CH₂)ₚO— | (c-5); |
| —O(CH₂)(CO)NR³— | (c-6); | —NR³(CO)(CH₂)O— | (c-7); |
| —(CH₂)ₙNR³(CO)— | (c-8); | —(CO)NR³(CH₂)ₙ— | (c-9); and |
| —N═CH(CO)NR³— | (c-10); | | |

wherein
m is 1 or 2;
n and p each independently represent 2 or 3;
each R³ is independently H or C₁₋₄alkyl;
R^{C} is selected from the group consisting of fluoro, methyl, hydroxy, methoxy, trifluoromethyl, and difluoromethyl;
R^{D} is selected from the group consisting of hydrogen, fluoro, methyl, hydroxy, methoxy, trifluoromethyl, and difluoromethyl; and
y represents 0, 1 or 2;
with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{D};
c) R^{D} is not hydroxy or methoxy when L^{A} is —O—, —OCH₂—, —CH₂O—, —NH—, —N(CH₃)—, —NH(CH₂)— or —(CH₂)NH—;
or a pharmaceutically acceptable addition salt or a solvate thereof.

2. The compound according to claim 1, wherein
R^{A} is a heteroaryl radical selected from the group consisting of pyridin-4-yl, pyrimidin-4-yl, and pyrazin-2-yl, each of which may be optionally substituted with 1, 2 or 3 substituents each independently selected from the group consisting of halo;
C₁₋₄alkyl optionally substituted with 1, 2, or 3 independently selected halo substituents; and
C₁₋₄alkyloxy optionally substituted with 1, 2, or 3 independently selected halo substituents.

3. The compound according to claim 1, wherein L^{A} is selected from the group consisting of a covalent bond, —CH₂—, —O—, —OCH₂—, —CH₂O—, and —NHCH₂—.

4. The compound of claim 1, wherein R^{B} is a bicyclic radical of formula (b-1) or (b-2).

5. The compound of claim 1, wherein R^{B} is a bicyclic radical of formula (b-1) or (b-2), wherein R¹ is selected from the group consisting of hydrogen, fluoro and methyl; R² is hydrogen; X¹ is N or CH; and X² is CH.

6. The compound claim 1, wherein R^{B} is a bicyclic radical of formula (b-1) or (b-2), wherein R¹ is selected from the group consisting of hydrogen, fluoro and methyl; R² is hydrogen; X¹ is N or CH; X² is CH; and —Y¹—Y²— forms a bivalent radical selected from the group consisting of (c-1), (c-2), (c-4), (c-6) and (c-9), wherein m is 2; n is 2 or 3; and p is 2.

7. The compound of claim 1, wherein R^{B} is selected from the group consisting of

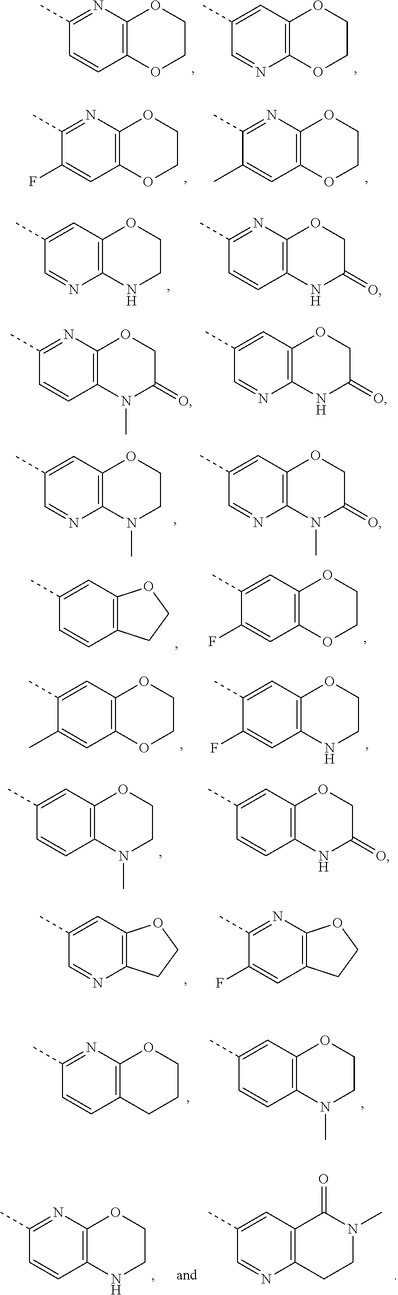

8. The compound of claim 1, wherein R^{D} is selected from the group consisting of hydrogen, fluoro, and methyl; and y represents 0 or 1.

9. A pharmaceutical composition comprising a prophylactically or a therapeutically effective amount of a compound of claim 1 and a pharmaceutically acceptable carrier.

10. (canceled)

11. (canceled)

12. (canceled)

13. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound of claim 1.

14. (canceled)
